(19) United States
(12) Patent Application Publication
(10) Pub. No.: US 2011/0021371 A1
(43) Pub. Date: Jan. 27, 2011

(54) DNA MICROARRAY BASED IDENTIFICATION AND MAPPING OF BALANCED TRANSLOCATION BREAKPOINTS

(75) Inventor: Harvey A. Greisman, Mercer Island, WA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: University of Washington, Seattle, WA (US)

(21) Appl. No.: 12/742,237

(22) PCT Filed: Nov. 10, 2008
(86) PCT No.: PCT/US08/83014

§ 371 Date: Oct. 7, 2010

Related U.S. Application Data
(60) Provisional application No. 60/986,576, filed on Nov. 8, 2007.

(51) Int. Cl. C40B 30/04; C12Q 1/68

(52) U.S. Cl. 506/9; 435/6

(57) ABSTRACT

Methods for detecting and mapping chromosomal rearrangements associated with various diseases using comparative genomic hybridization are disclosed. Included are methods to identify translocation partners of known genomic loci and to determine translocation breakpoints. These methods may be used in the prognosis, diagnosis, and determination of predisposition to diseases that involve chromosomal rearrangements.

### STATEMENT AS TO RIGHTS TO INVENTIONS MADE UNDER FEDERALLY SPONSORED RESEARCH AND DEVELOPMENT

This invention was made with Government support under NCI Grant No. 5P30 CA015704. The Government has certain rights in this invention.

### CROSS-REFERENCES TO RELATED APPLICATIONS NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

Large scale genomic aberrations, including balanced rearrangements (translocations and inversions) and genomic imbalances (deletions, duplications, and amplifications) are common in cancer and play central roles in oncogenesis. Genomic deletions typically have been associated with loss of tumor suppressor gene function, amplifications with over-expression of proto-oncogenes, and translocations with the creation of novel oncogenic gene fusions or deregulated oncogene expression. Historically, balanced translocations and gene fusions have been seen predominantly in hematologic and mesenchymal tumors, including sarcomas, leukemias, and lymphomas (Rabbits, Nature 372(6502):143 (1994)), and less commonly in epithelial tumors such as carcinomas. More recently, oncogenic gene fusions have been identified in carcinomas of the prostate (Tomlins, Rhodes, et al., Science 310(5748):644-648 (2005)), thyroid (Bongarzone, Butti et al., Cancer Res. 54(11):2979-2985 (1994); Kroll, Sarraf, et al., Science 289(5483):1357-1360 (2000)), and lung (Soda, Choi, et al., Nature 448(7153):561 (2007)), suggesting that they are more common in carcinomas than previously appreciated, possibly because of the cytogenetic complexity of these tumors or for other technical reasons (Mitelman, Johansson, et al., Nat Genet 36(4):331 (2004)).

Analysis of large scale aberrations has transformed our understanding of oncogenesis through the identification of novel proto-oncogenes, tumor suppressor genes, and oncogenic gene fusions. Advances in microarray-based comparative genomic hybridization (array-CGH/aCGH) technology have led to exponential accumulation of data on genomic imbalances in cancer, which can be mapped at ever increasing resolution. A minority of translocations have small deletions at one or both breakpoints that can be identified by arrayCGH. However, such imbalances are not usually detectable and, even when present, they do not indicate the identity of the other partner gene.

Structural variation is now recognized as an important source of genetic variation across human populations (Bansal, Bashir, et al., Genome Research 17(2):219-230 (2007); Korbel, Urban, et al., Science 318(5849):420-426 (2007); Hurles, Dermitzakis, et al., Trends in Genetics 24(5):238-245 (2008); Kidd, Cooper, et al., Nature 453(7191:56-64 (2008)). Copy number variants (CNV) like chromosomal deletions or segmental duplications represent the most common structural variants reported to date and have been associated with human disease or predisposition to disease (Gonzalez, Kulkarni, et al., Science 30(5714):1434-1440 (2005); Hollox, Huffmeier, et al., Nat Genet 40(1):23 (2008)). Methods for identifying balanced chromosomal rearrangements have lagged behind those for detecting CNV, but chromosomal inversions and more complex rearrangements are increasingly recognized as important sources of structural and functional variation and also have been associated with human disease (Feuk, MacDonald, et al., PLoS Genetics 1(4):e56 (2005); Turner, Shendure, et al., Nat Meth 3(6):439 (2006); Bansal, Bashir, et al., Genome Research 17(2):219-230 (2007); Flores, Morales, et al., PNAS 104(15):6099-6106 (2007); Kidd, Cooper, et al., Nature 453(7191:56-64 (2008)).

Currently, methods designed to detect balanced rearrangements are limited when compared with methods available to detect genomic imbalances. Traditional cytogenetic analysis and multicolor (or spectral) karyotyping are powerful genome-wide techniques for identifying large-scale genomic abnormalities, but these methods are laborious, require growth of cells in culture, and have limited resolution (˜5 million bp). Fluorescence in situ hybridization (FISH) can be used to analyze small numbers of genomic loci at higher resolution (typically 100-1000 kb), but FISH is not readily scalable and requires prior knowledge of the fusion partners. In array painting, (Fiegler, Gribble, et al., J Med Genet 40(9):664-670 (2003)) DNA is amplified from flow-sorted abnormal chromosomes and hybridized to CGH arrays, enabling translocation breakpoints to be mapped to high resolution (Gribble, Kalaitzopoulos, et al., J Med Genet 44(1):51-58 (2007)). This technology, however, is not widely available and is limited to cells that can be grown in culture.

Further complicating the identification and characterization of chromosomal translocations is the fact that, increasingly, genes involved in translocations are recognized to be “promiscuous” in that they can be found fused to a variety of partner genes in different translocations and different types of tumors (Cleary, N Engl J Med 329(13):958-959 (1993)). Striking examples include the mixed lineage leukemia (MLL) gene (Meyer, Schneider, et al., Leukemia 20(5):777 (2006)), the immunoglobulin heavy chain (IgH) locus (Willis and Dyer, Blood 96(3):808-822 (2000), and ETV6 (Bohlander, Seminars in Cancer Biology 15(3):162-174 (2005)), each of which is capable of partnering with 20 or more different genomic loci in various translocations. Consequently, a variety of molecular methods have been developed to identify unknown fusion partner genes in balanced translocations when one of the partners is known or can be surmised. These techniques include rapid amplification of cDNA ends (RACE) (Frohman, Dush, et al., PNAS 85(23):8998-9002 (1988)), long-distance inverse (LDI) PCR (Ochman, Gerber, et al., Genetics 120(3):621-623 (1988); Willis, Jadayel, et al., Blood 90(6):2456-2464 (1997)), and array-based detection of fusion transcripts (Nasedkina, Domer, et al., Haematologica 87(4):363-72 (2002); Maroc, Morel, et al., Leukemia 18(9):1522-30 (2004)). Again, these techniques are all laborious, have limited throughput and are not suitable for the routine analysis of clinical samples.

Thus, improved methods that allow the routine detection of chromosomal abnormalities, particularly balanced translocations are needed. The present invention satisfies these and other needs.

### BRIEF SUMMARY OF THE INVENTION

Although Comparative Genome Hybridization (CGH) methods have proven to be powerful for the detection of chromosomal imbalances, previous CGH methods have been generally incapable of detecting balanced genomic rearrangements like reciprocal translocations, which play a prominent role in the pathogenesis and diagnosis of various cancers, including lymphomas and leukemias, among other tumors. The inability of previous CGH methods to detect balanced translocations is due at least in part to the fact that these methods rely on the detection of relative differences between test and reference samples, while balanced translocations result in no net loss or gain of chromosomal material, and thus, the same relative quantities are maintained.

To overcome these limitations and extend the range of chromosomal abnormalities that can be detected, we have developed translocation CGH (tCGH), a method that can identify balanced translocation breakpoints at ultra-high resolution. The present invention is based in part on the use of primers specific to sequences in known genomic loci in linear amplification reactions to generate probes that span the sequence of the known genomic locus and a translocation partner. The pattern and extent of hybridization of a probe generated from the test sample as compared to the hybridization of a similar probe derived from a reference sample allows the identification of the translocation partner of the known genomic locus. The use of high density microarrays, such as tiling density microarrays, allows high resolution mapping of the breakpoints of the translocation.

As described in greater detail below, we demonstrate the ability of tCGH to detect the most common types of IgH translocations, including those occurring at joining (J_{H}) segments and within repetitive switch recombination (S_{H}) regions. Known translocation breakpoints were identified in each cell line analyzed, including BCL2, BCL6, cyclin D1 (CCND1), and MYC translocations as well as complex and cryptic IgH rearrangements involving these loci. The utility of tCGH is further demonstrated by mapping and cloning novel CCND1 breakpoints in 5 mantle cell and prolymphocytic lymphomas, the largest such series reported to date. Additionally, multiplex tCGH analysis is used to detect several common translocations associated with various myeloid leukemias.

Accordingly, in one embodiment, the invention provides a method of determining a chromosomal rearrangement at a known genomic locus in a test sample by (a) isolating a first genomic DNA from cells of a test sample and a second genomic DNA from cells of a reference sample, (b) performing linear amplification and labeling of the first genomic DNA sample using a primer specific for a known DNA sequence within the known genomic locus to generate an amplified test DNA product comprising a first detectable label; and performing linear amplification and labeling of the second genomic DNA sample using the primer specific for a known DNA sequence within known genomic locus to generate an amplified reference DNA product comprising a second detectable label, (c) hybridizing the amplified test and reference DNA products to a DNA microarray comprising genomic DNA sequences, and (d) comparing the pattern and extent of hybridization of the test amplified DNA product with the reference amplified DNA product to the DNA microarray, where excess hybridization of the linear amplified test sample DNA product over the linear amplified reference sample DNA product to a DNA microarray element distinct from that of the known genomic locus is indicative of a rearrangement of the known genomic locus with a second genomic locus in the cell.

In a second embodiment, the present invention provides a method of identifying a chromosomal rearrangement partner of a known genetic locus in a test sample by (a) isolating a first genomic DNA from cells of a test sample and a second genomic DNA from cells of a reference sample, (b) performing linear amplification and labeling of the first genomic DNA sample using a primer specific for a known DNA sequence within known genomic locus to generate an amplified test DNA product comprising a first detectable label; and performing linear amplification and labeling of the second genomic DNA sample using the primer specific for a known DNA sequence within known genomic locus to generate an amplified reference DNA product comprising a second detectable label, (c) hybridizing the labeled and amplified test and reference DNA products to a DNA microarray comprising genomic DNA sequences, and (d) comparing the pattern and extent of hybridization of the test amplified DNA product with the reference amplified DNA product to the DNA microarray, where excess hybridization of the linear amplified test sample DNA product over the linear amplified reference sample DNA product to a DNA microarray element distinct from that of the known genomic locus identifies the element of the DNA microarray as a rearrangement partner of the known genomic locus.

In a third embodiment, the present invention provides a method of simultaneously determining chromosomal rearrangements, as well as chromosomal translocation at known genomic locus of a test sample by (a) isolating a first genomic DNA from cells of a test sample and a second genomic DNA from cells of a reference sample, (b) performing linear amplification of the first genomic DNA using a primer specific for a known DNA sequence within known genomic locus to generate a mixture of test genomic DNA and primer specific, amplified test DNA product; and performing linear amplification of the second genomic DNA using the same specific primer for a known DNA sequence within known genomic locus sample to generate a mixture of reference genomic DNA and primer specific, amplified reference DNA product, (c) further amplifying and labeling the test and reference sample mixtures via oligonucleotide primed, polymerase mediated extension, (d) hybridizing the labeled and amplified test and reference DNA product to a DNA microarray comprising genomic DNA sequences, and (e) comparing the pattern and extent of hybridization of the test amplified DNA product with the reference amplified DNA product to the DNA microarray, where (i) a greater extent of hybridization of the test amplified DNA product to an element of the DNA microarray as compared to the extent of hybridization of the reference amplified DNA product to the element of the DNA microarray, when both hybridize to the element, indicates an amplification of the DNA sequence represented by the element of the microarray in the test sample, (ii) hybridization of the reference amplified DNA product to an element of the DNA microarray in excess of hybridization of the test amplified DNA product to the element of the DNA microarray indicates a deletion of the DNA sequence represented by the element of the microarray in the test sample, and (iii) hybridization of the test amplified DNA product to a DNA array element distinct from that of the known genomic locus in excess of hybridization of the reference amplified DNA product to the DNA array element is indicative of a translocation of the known genomic locus with a second genomic locus in the cell.

In an aspect of the above embodiments, the method includes the further step of determining the last element in a series of elements corresponding to the linear sequence of the known genomic locus that hybridizes to the amplified DNA product, thereby identifying the approximate location of the rearrangement breakpoint of the known genomic locus.

In another aspect of the above embodiments, the method includes the further step of determining the first element in a series of elements, corresponding to the linear sequence of a second genomic locus distinct from that of the known genomic locus, that hybridizes to the amplified DNA product, thereby identifying the approximate location of the rearrangement breakpoint of the translocation partner.

In additional aspects of the above embodiments, the test and reference samples comprise the same genomic DNA, and the test sample, but not the reference sample, is subjected to the linear amplification step of part (b).

In further additional aspects of the above embodiments, the first and second detectable labels are the same and the hybridizing of the amplified test and reference DNA products is to separate but identical microarrays or sequentially to the same microarray.

In yet another particular embodiment, the methods of the present invention comprise the amplification and detection of only a first sample DNA, which is then compared to a predetermined reference reading or detection.

In a fourth embodiment, the present invention provides a method of determining a chromosomal rearrangement in a test sample by (a) isolating a first genomic DNA from cells of a test sample and a second genomic DNA from cells of a reference sample; (b) performing linear amplification of the first genomic DNA sample using a primer specific for a known DNA sequence within a known genomic locus to generate an amplified test DNA product (T+); performing linear amplification and labeling of the second genomic DNA sample using the primer specific for a known DNA sequence within the known genomic locus to generate an amplified reference DNA product (N+); performing a mock linear amplification of the first genomic DNA sample by omitting the primer specific for a known DNA sequence within a known genomic locus to generate a mock test DNA product (T−); performing a mock linear amplification of the second genomic DNA sample by omitting the primer specific for a known DNA sequence within a known genomic locus to generate a mock reference DNA product (N−); (c) labeling each of T+, N+, T−, and N− with a different detectable label by primer extension using random primers; (d) co-hybridizing T+ and N+ to a first DNA microarray comprising genomic DNA sequences; (e) co-hybridizing T− and N− to a second DNA microarray comprising genomic DNA sequences; (f) comparing the pattern and extent of hybridization signal on the first DNA microarray with the pattern and extent of hybridization signal on the second DNA microarray; wherein a right triangular pattern of hybridization signal on a scatter plot of hybridization signal plotted against chromosomal position from the first microarray in the absence of a similar pattern from the second microarray is indicative of a chromosomal translocation with the vertical leg marking a chromosomal translocation breakpoint; and wherein a rectangular pattern of hybridization signal on a scatter plot of hybridization signal plotted against chromosomal position at the same position from the first microarray and the second microarray is indicative of a chromosomal duplication or deletion with the vertical legs marking the two end points of a duplicated or deleted genomic region, thereby providing a determination of a chromosomal rearrangement in the test sample.

In a fifth embodiment, the present invention provides a method of determining a chromosomal rearrangement in a test sample by (a) isolating a first genomic DNA from cells of a test sample and a second genomic DNA from cells of a reference sample; (b) performing linear amplification of the first genomic DNA sample using a primer specific for a known DNA sequence within a known genomic locus to generate an amplified test DNA product (T+); performing linear amplification and labeling of the second genomic DNA sample using the primer specific for a known DNA sequence within the known genomic locus to generate an amplified reference DNA product (N+); performing a mock linear amplification of the first genomic DNA sample by omitting the primer specific for a known DNA sequence within a known genomic locus to generate a mock test DNA product (T−); performing a mock linear amplification of the second genomic DNA sample by omitting the primer specific for a known DNA sequence within a known genomic locus to generate a mock reference DNA product (N−); (c) labeling each of T+, N+, T−, and N− with a different detectable label by primer extension using random primers; (d) co-hybridizing T+ and T− to a first DNA microarray comprising genomic DNA sequences; (e) co-hybridizing N+ and N− to a second DNA microarray comprising genomic DNA sequences; (f) comparing the pattern and extent of hybridization signal on the first DNA microarray with the pattern and extent of hybridization signal on the second DNA microarray; wherein a right triangular pattern of hybridization signal on a scatter plot of hybridization signal plotted against chromosomal position from the first microarray in the absence of a similar pattern from the second microarray is indicative of a chromosomal translocation with the vertical leg marking a chromosomal translocation breakpoint; and wherein patterns of hybridization signal on a scatter plot of hybridization signal plotted against chromosomal position common to both the first microarray and the second microarray indicate pseudo-breakpoints, thereby providing a determination of a chromosomal rearrangement in the test sample.

In a sixth embodiment, the present invention provides a method of diagnosing a disease in a subject, where the disease results from a chromosomal rearrangement by (a) obtaining a biological sample from the subject; (b) isolating a first genomic DNA from cells of the biological sample and a second genomic DNA from cells of a reference sample; (c) performing linear amplification and labeling of the first genomic DNA sample using a primer specific for a known DNA sequence within a known genomic locus associated with the disease to generate an amplified test DNA product comprising a first detectable label; and performing linear amplification and labeling of the second genomic DNA sample using the primer specific for a known DNA sequence within the known genomic locus to generate an amplified reference DNA product comprising a second detectable label; (d) hybridizing the amplified test and reference DNA products to a DNA microarray comprising genomic DNA sequences; and (e) comparing the pattern and extent of hybridization of the test amplified DNA product with the reference amplified DNA product to the DNA microarray; wherein excess hybridization of the linear amplified test sample DNA product over the linear amplified reference sample DNA product to a DNA microarray element distinct from that of the known genomic locus identifies the element of the DNA microarray as a rearrangement partner of the known genomic locus and the identity of the rearrangement partner provides a diagnosis of the disease in the subject.

In some aspects of the above embodiments, the chromosomal rearrangement is a translocation. In other aspects of the above embodiments, the chromosomal rearrangement is a chromosomal inversion or an insertion of a DNA fragment derived from one chromosomal locus into a second, distinct chromosomal locus. In other embodiments of the invention, the methods further comprise the detection of a chromosomal abnormality selected from a deletion, a duplication, an amplification, and an inversion. In certain embodiments, the detection of more than one type of chromosomal abnormality is performed simultaneously. In other embodiments, the detection of more than one type of chromosomal abnormality is performed sequentially.

In other aspects of the above embodiments, the first and second detectable labels are incorporated during amplification or else incorporated after amplification.

In other aspects of the above embodiments, the first and second detectable label are fluorescent labels which can include Cy3 and Cy5.

In further aspects of the above embodiments, the DNA microarray is a tiling density DNA microarray.

In another aspect of the above embodiments, the known genomic locus corresponds to an immunoglobulin gene.

In another embodiment of the methods of the invention, the known genomic locus corresponds to a loci that is associated with a particular disease or disease state. In certain embodiments, the disease is cancer. In a particular embodiment, the cancer is a leukemia, such as a myeloid leukemia.

In some aspects of the above embodiments, the cell of the test sample is a tumor cell and of the reference sample is a normal cell, where the tumor cell is a lymphoma or leukemia.

In some aspects of the above embodiments the cell of the test sample is a cell, normal or abnormal, from one individual and the reference sample is a cell, normal or abnormal, from a second individual, and the chromosomal rearrangement is a translocation, inversion, deletion, duplication, insertion, or other complex rearrangement that is present in the test sample but not in the reference sample, or is present in the reference sample but not in the test sample.

### BRIEF DESCRIPTION OF THE DRAWINGS

FIG. 1 illustrates: (a) IgH locus showing J_{H} and switch repeat regions; (b) Linear amplification using J_{H} primer; (c) Linear amplification using S₅ (S_{ναε}) primer; (d) Outline of a typical Translocation CGH (tCGH) experiment.

FIG. 2 illustrates tCGH data for cell lines with known IgH translocation breakpoints. (a) J_{H}-BCL2 breakpoint (minor cluster region) in DHL16 cell line; (b) J_{H}-MYC breakpoint in MC116 cell line; (c) S_{α}-CCND1 breakpoint in U266 cell line; (d) S_{γ}-BCL6 breakpoint in OCI-Ly8 cell line.

FIG. 3 illustrates (a) the analysis of a J_{H}-BCL2 breakpoint and BCL2 deletion in the RL7 cell line: (i) RL7+J_{H} (Cy3)/Normal+J_{H} (Cy5)—breakpoint and deletion; (ii) RL7−J_{H} (Cy3)/Normal−J_{H} (Cy5)—deletion only; (iii) RL7+J_{H} (Cy3)/RL7−J_{H} (Cy5)—breakpoint only. Part (b) illustrates the overlay of RL7/BCL2 array data for all three experiments above. Part (c) illustrates the analysis of a J_{H}-CCND1 breakpoint and CCND1 duplication/deletion in MO2058 cell line: (i) MO2058+J_{H} (Cy3)/Normal+J_{H} (Cy5)—breakpoint and duplication/deletion; (ii) MO2058−J_{H} (Cy3)/Normal−J_{H} (Cy5)—duplication/deletion only; (iii) MO2058+J_{H} (Cy3)/Granta−J_{H} (Cy5)—breakpoint only. Part (d) illustrates the overlay of MO2058/CCND1 array data for all three experiments above

FIG. 4 illustrates multiple IgH breakpoints identified in OCI-Ly8 cell line: (a) J_{H}-BCL2—“der(14)” breakpoint; (b) S_{γ3}-BCL6—“der(3)” breakpoint identified using the S_{γ}R primer; (c) S_{γ}-MYC—“der(8)” breakpoint identified using the S_{γ}R primer; (d) S_{γ}-BCL6—“der(14)” breakpoint identified using S_{γ}F primer.

FIG. 5 illustrates the effect on breakpoint profile of linear amplification extension time of 6 minutes (light line) versus 10 minutes (dark line). Part (a) illustrates the S_{γ}-BCL6 breakpoint in OCI-Ly8 cell line; Part (b) illustrates the S_{γ}-BCL6 breakpoint in OCI-Ly8 cell line.

FIG. 6 illustrates tCGH analysis of 5 primary mantle cell lymphomas showing different J_{H}-CCND1 breakpoints.

FIG. 7 provides an overview of a typical Translocation CGH (tCGH) experiment.

FIG. 8 provides an overview of typical IgH translocations that involve a partner loci in various B cell lymphomas and plasma cell myelomas, which were used as a model system for the establishment and validation of the tCGH system.

FIG. 9 illustrates the set-up for tCGH detection of VDJ-associated translocations with IgH breakpoints on the der(14) chromosome and reciprocal breakpoints in a number of D_{H} segments.

FIG. 10 illustrates tCGH analysis of reciprocal J_{H}-BCL2 (a) and BCL2-D_{H} (b) fusions that map to the BCL2 minor translocation cluster; both reciprocal S_{γ3}-BCL6 fusions (c and d) found in the OCI-Ly8 lymphoma cell line; a non-IgH BCL6 exon 1 rearrangement (e) with an inverted orientation; an IgH-MYC fusion in the Burkitt lymphoma cell line MC116 (f); and several other MYC rearrangements (g-i), including a non-IgH MYC rearrangement (i) with an inverted orientation.

FIG. 11 illustrates tCGH analysis of copy number changes in a novel 167 kb interstitial deletion within the large (190 kb) intron of BCL2 using a number of different linear amplification schemes (a-e).

FIG. 12 illustrates the identification, by tCGH analysis, of novel CCDN1 breakpoints in primary lymphoma, from five primary MCL cases having non-MTC breakpoints (a-e).

FIG. 13 illustrates the identification, by tCGH analysis, of duplications that span the CCND1 gene and extent precisely to the respective JH-CCND1 breakpoint junctions in both MO2058 (a-c) and Granta (d-f) cell lines.

FIG. 14 illustrates the identification, by tCGH analysis, of an approximately 6 kb deletion at the IgH-BCL2 breakpoint in the OCI-Ly8 lymphoma cell line.

FIG. 15 illustrates the identification, by tCGH analysis, of a novel cryptic insertion into the CCND1 locus of a ˜100 kb IgH constant region segment that extends from S_{α1} to S_{γ4} and encompasses the 3′ α1 enhancer, using both SγR (a) and SₚF (b) primed linear amplification. Off-target amplification of sequences away from expected translocation breakpoints is illustrated when mock amplified tumor DNA is used as a hybridization control (c), and when normal genomic DNA is analyzed (d).

FIG. 16 illustrates off-target amplification of sequences away from expected translocation breakpoints in MO2058 (a) and Granta (b) cell lines when mock amplified tumor DNA is used as a hybridization control. Similar results are seen when normal genomic DNA is analyzed (c-f).

FIG. 17 illustrates the determination of the analytic sensitivity of tCGH analysis by mixing equal amounts of DHL16, RL7, and Granta 519 genomic DNA (designated “33% dilution”); 20% and 15% dilution samples were produced by mixing with normal genomic DNA. Samples were then amplified for 12 or 20 cycles using the J_{H} primer and co-hybridized to similarly amplified normal genomic DNA.

FIG. 18 illustrates the results of multiplex linear amplification and tCGH analysis of three chronic myeloid leukemia cell lines, characterized by BCR-ABL balanced translocations t(22;9), using the myeloid primer mix (MPM) and AML pilot array.

FIG. 19 illustrates the results of multiplex linear amplification and tCGH analysis of two acute promyelocytic leukemia (APL) cell lines characterized by PML-RARA balanced translocations t(15;21) (top panel) and two acute myelomonocytic leukemia/eosinophilia cell lines characterized by MYH11-CBFB fusions caused by a chromosomal inversion inv(16) (bottom panel), using the myeloid primer mix (MPM) and AML pilot array.

FIG. 20 illustrates the results of multiplex linear amplification and tCGH analysis of an MLL leukemia cell line characterized by an AF9-MLL balanced translocation t(9;11) (top panel) using the P1/P7 primer mix (MPM) and AML pilot array, and of a Kasumi Acute Myeloid Leukemia cell line characterized by an ETO-AML1 balanced translocations t(8;21) (bottom panel), using the 821 primer mix (MPM) and AML pilot array.

### DETAILED DESCRIPTION OF THE INVENTION

Array based comparative genomic hybridization (CGH) has revolutionized the study of chromosomal imbalances but generally is incapable of detecting balanced genomic rearrangements like reciprocal translocations, which play central roles in the pathogenesis and diagnosis of lymphomas, leukemias and other tumors. The precise identification of immunoglobulin heavy chain (IgH) translocation partners, for example, is essential for the classification of B cell lymphomas and for predicting prognosis in plasma cell neoplasms like multiple myeloma.

Using IgH translocations as a model for balanced genomic rearrangements, we have developed a method of array CGH that we call translocation-CGH (tCGH) which enables the rapid identification of IgH translocation partners and precise mapping of translocation-associated breakpoints to unprecedented resolution. As described in greater detail below, to render IgH translocations detectable on CGH arrays, genomic DNA from test and reference samples is modified prior to array hybridization in an enzymatic linear amplification reaction that employs a single IgH joining (J_{H}) or switch (Sμ/Sα/Sε) region primer, resulting in specific amplification of any fusion partner sequences that may be inserted (via translocation or other rearrangement) downstream of the IgH primer. Using a single tiling-density oligonucleotide array representing such common IgH partner loci as MYC, BCL2 and CCND1 (cyclin D1), tCGH successfully identified and mapped to ˜100 bp resolution an assortment of known IgH fusion breakpoints in various cell lines and primary lymphomas, including J_{H}-CCND1 breakpoints in MO2058 and Granta 519 cell lines (mantle cell lymphoma), a cytogenetically cryptic Sα-CCND1 fusion in U266 (myeloma), J_{H}-MYC and S μ-MYC breakpoints in MC116 and Raji (Burkitt lymphoma), and J_{H}-BCL2 breakpoints in DHL16 (large cell lymphoma; minor cluster region) and in an archival case of follicular lymphoma (major breakpoint region).

We then used tCGH to analyze 4 archival cases of mantle cell lymphoma and one t(11;14)-positive case of B cell prolymphocytic leukemia, all of which lacked PCR-detectable translocation breakpoints at the CCND1 major translocation cluster (MTC). Five novel CCND1 translocation breakpoints were identified and mapped to ˜100 bp resolution, allowing the rapid design of patient-specific PCR primers for amplification, sequencing, and confirmation of the predicted breakpoints. One breakpoint mapped to within 500 bp of the MTC, whereas the other 4 were scattered across a ˜150 kb region flanking the MTC. To our knowledge, this represents the largest series of non-MTC mantle cell lymphoma breakpoint sequences reported to date. These results also illustrate how tCGH can facilitate the rapid cloning of previously unidentified IgH translocation breakpoints dispersed over very large genomic regions. Because tCGH requires only genomic DNA and can simultaneously detect both balanced IgH translocations and genomic imbalances at ultra-high resolution on the same array, it can be a useful alternative to molecular cytogenetic methods (e.g. FISH) for clinical testing of B cell and plasma cell neoplasms. tCGH also will facilitate the development of highly sensitive breakpoint-specific PCR assays for detecting minimal residual disease. Finally, because the primer used in the linear amplification reaction is fully customizable, tCGH can readily be adapted to identify and map other balanced translocations (or more complex genomic fusions) that involve non-IgH loci, provided that one of the fusion partners is known.

In one embodiment, the present invention provides a method of detecting a chromosomal rearrangement, the method comprising the steps of: (a) amplifying a target genomic locus; (b) hybridizing said amplified product to a nucleic acid array; and (c) comparing said hybridization pattern to a reference, wherein said amplification is linear amplification, and wherein differential hybridization of the amplified genomic locus as compared to the reference indicates the presence of a genomic rearrangement. In certain embodiments, the genomic rearrangement is a balanced rearrangement, such as a balanced translocation or inversion.

In one embodiment, the present invention provides a method of detecting a balanced chromosomal translocation. In certain embodiments, the methods of the invention comprise the steps of: (a) amplifying a target genomic locus; (b) hybridizing said amplified product to a nucleic acid array; and (c) comparing said hybridization pattern to a reference, wherein said amplification is linear amplification, and wherein the presence of a right triangular hybridization pattern indicates the presence of a balanced chromosomal translocation. In certain embodiments of the invention, said right triangular hybridization pattern comprises an asymmetric hybridization pattern. In certain embodiments, the methods of the present invention may comprise the detection and/or mapping of breakpoints in both partner loci involved in a chromosomal translocation. In yet other embodiments, the methods of the present invention comprise the detection of a chromosomal rearrangement other than a balanced translocation.

In certain embodiments of the methods provided above, multiplex linear amplification is used to amplify more than one amplicon. In particular embodiments, said methods comprise the simultaneous survey of more than one genomic locus.

In particular embodiments, a plurality of amplification primers is used in the methods of the present invention. Said plurality of amplification primers may comprise primers for the amplification of loci implicated in balanced translocations associated with a disease. Any disease associated with a balanced chromosomal translocation may be detected by the methods of the present invention. In a particular embodiment of the invention, the disease is cancer, such as a lymphoma or a leukemia. In specific embodiments of the invention, a plurality of primers selected from the MPM mix, the 821 mix, the P1/P7 mix, and a plurality of D_{H} primers may be used in the methods provided herein.

In certain embodiments of the invention, the array used to detect the products of linear amplification may comprise a microarray or high density tiled array. In some embodiments, said array may comprise probes to a plurality of genomic loci. In certain embodiments, at least one genomic loci corresponding to a probe on an array of the invention may be associated with a disease. In particular embodiments, the disease may be cancer, such as a lymphoma or a leukemia. In a specific embodiment, said array may comprise an AML pilot array.

In another embodiment, the methods of the present invention may further comprise the detection of a second chromosomal rearrangement selected from a duplication, an amplification, a deletion, an inversion, a balanced translocation, and an unbalanced translocation. In certain embodiments, the detection of a first rearrangement and a second rearrangement may be sequential or simultaneous. In a particular embodiment, the methods of the present invention comprise the simultaneous detection of both a balanced rearrangement and an unbalanced rearrangement. Said balanced and unbalanced rearrangements may be present at the same genomic locus or in different genetic loci.

In yet other embodiments, the present invention provides novel kits for use in the detection of a balanced chromosomal translocation. In certain embodiments, the kits of the present invention comprise a primer for the linear amplification of a locus implicated in a translocation. In other embodiments, a kit of the invention may comprise an array for the detection of a linear amplification product from a locus implicated in a translocation. In certain embodiments of the invention, a kit may comprise a plurality of primers for the amplification of loci implicated in translocations. In yet other embodiments, the kits of the present invention may find use in the diagnosis or prognosis of a disease associated with a chromosomal translocation. In a specific embodiment, the disease may be cancer, such as a lymphoma or a leukemia.

### 1. DEFINITIONS

The terms “chromosomal rearrangement” or “chromosomal abnormality” refer generally to the aberrant joining of segments of chromosomal material in a manner not found in a wild-type or normal cell. Examples of chromosomal rearrangements include deletions, amplifications, inversions, or translocations. Chromosomal rearrangements can arise after spontaneous breaks occur in a chromosome. If the break or breaks result in the loss of a piece of chromosome, a deletion has occurred. An inversion results when a segment of chromosome breaks off, is reversed (inverted), and is reinserted into its original location. When a piece of one chromosome is exchanged with a piece from another chromosome a translocation has occurred. Amplification results in multiple copies of particular regions of a chromosome. Chromosomal rearrangements may also encompass combinations of the above.

The term “translocation” or “chromosomal translocation” refers generally to an exchange of chromosomal material between the same or different chromosomes in equal or unequal amounts. Frequently, the exchange occurs between nonhomologous chromosomes.

A “balanced” translocation refers generally to an exchange of chromosomal material in which there is no net loss or gain of genetic material.

An “unbalanced” translocation refers generally to an unequal exchange of chromosomal material resulting in extra or missing chromosomal material.

A “nucleic acid array” or “nucleic acid microarray” is a plurality of nucleic acid elements, each comprising one or more target nucleic acid molecules immobilized on a solid surface to which probe nucleic acids are hybridized. Nucleic acids molecules that can be immobilized on such solid support include, without limitation, oligonucleotides, cDNAs, and genomic DNA. In the context of the present invention, microarrays containing sequences corresponding to different segments of genomic nucleic acids are used. The genomic elements of microarrays can represent the entire genome of an organism or else represent defined regions of a genome, e.g., particular chromosomes or contiguous segments thereof.

Genome tiling microarrays comprise overlapping oligonucleotides designed to provide complete or nearly complete representation of an entire genomic region of interest.

Comparative genomic hybridization (CGH) refers generally to molecular-cytogenetic methods for the analysis of copy number changes (gains/losses) in the DNA content of a given subject's DNA and often in tumor cells. In the context of cancer, the method is based on the hybridization of labeled tumor DNA (frequently with a fluorescent label) and normal DNA (frequently with a second, different fluorescent label) to normal human metaphase preparations. Using epifluorescence microscopy and quantitative image analysis, regional differences in the fluorescence ratio of gains/losses vs. control DNA can be detected and used for identifying abnormal regions in the genome. CGH will generally detect only unbalanced chromosomes changes. Structural chromosome aberrations such as balanced reciprocal translocations or inversions can not be detected, as they do not change the copy number. See, e.g., Kallioniemi et al., Science 258: 818-821 (1992).

In a variation of CGH, termed “Chromosomal Microarray Analysis (CMA)” or “ArrayCGH”, DNA from subject tissue and from normal control tissue (a reference) is differentially labeled (e.g., with different fluorescent labels). After mixing subject and reference DNA along with unlabeled human cot 1 DNA to suppress repetitive DNA sequences, the mixture is hybridized to a slide containing a plurality of defined DNA probes, generally from a normal reference cell. See, e.g., U.S. Pat. Nos. 5,830,645; 6,562,565. When oligonucleotides are used as elements on microarrays, a resolution typically of 20-80 base pairs can be obtained, as compared to the use of BAC arrays which allow a resolution of 100 kb. The (fluorescence) color ratio along elements of the array is used to evaluate regions of DNA gain or loss in the subject sample.

The term “right triangular pattern of hybridization” or “right triangular hybridization pattern” refers generally to an asymmetric pattern on a plot of the hybridization signal (or of the hybridization signal ratio or its logarithm) versus the chromosomal position, including any asymmetric hybridization signal pattern that is characterized by (i) a single discrete boundary, which marks the rearrangement breakpoint, and (ii) the gradual return of the hybridization signal (or its ratio or log-ratio) to the baseline in the direction of either the centromere or telomere, which results in a second boundary that is not discrete.

“Amplification” or an “amplification reaction” refers to any chemical reaction, including an enzymatic reaction, which results in increased copies of a template nucleic acid sequence. Amplification reactions include polymerase chain reaction (PCR) and ligase chain reaction (LCR) (see U.S. Pat. Nos. 4,683,195 and 4,683,202; PCR Protocols: A Guide to Methods and Applications (Innis et al., eds, 1990)), strand displacement amplification (SDA) (Walker, et al. Nucleic Acids Res. 20(7):1691 (1992); Walker PCR Methods Appl 3(1):1 (1993)), transcription-mediated amplification (Phyffer, et al., J. Clin. Microbiol. 34:834 (1996); Vuorinen, et al., J. Clin. Microbiol. 33:1856 (1995)), nucleic acid sequence-based amplification (NASBA) (Compton, Nature 350(6313):91 (1991), rolling circle amplification (RCA) (Lisby, Mol. Biotechnol. 12(1):75 (1999)); Hatch et al., Genet. Anal. 15(2):35 (1999)) and branched DNA signal amplification (bDNA) (see, e.g., Iqbal et al., Mol. Cell Probes 13(4):315 (1999)).

Linear amplification refers to an amplification reaction which does not result in the exponential amplification of DNA. Examples of linear amplification of DNA include the amplification of DNA by PCR methods when only a single primer is used, as described herein. See, also, Liu, C. L., S. L. Schreiber, et al., BMC Genomics, 4: Art. No. 19, May 9, 2003. Other examples include isothermic amplification reactions such as strand displacement amplification (SDA) (Walker, et al. Nucleic Acids Res. 20(7):1691 (1992); Walker PCR Methods Appl 3(1):1 (1993), among others.

The reagents used in an amplification reaction can include, e.g., oligonucleotide primers; borate, phosphate, carbonate, barbital, Tris, etc. based buffers (see, U.S. Pat. No. 5,508,178); salts such as potassium or sodium chloride; magnesium; deoxynucleotide triphosphates (dNTPs); a nucleic acid polymerase such as Taq DNA polymerase; as well as DMSO; and stabilizing agents such as gelatin, bovine serum albumin, and non-ionic detergents (e.g. Tween-20).

A “probe” refers generally to a nucleic acid that is complementary to a specific nucleic acid sequence of interest.

The term “primer” refers to a nucleic acid sequence that primes the synthesis of a polynucleotide in an amplification reaction. Typically a primer comprises fewer than about 100 nucleotides and preferably comprises fewer than about 30 nucleotides. Exemplary primers range from about 5 to about 25 nucleotides.

A “target” or “target sequence” refers to a single or double stranded polynucleotide sequence sought to be amplified in an amplification reaction.

The phrase “nucleic acid” or “polynucleotide” refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

Two nucleic acid sequences or polypeptides are said to be “identical” if the sequence of nucleotides or amino acid residues, respectively, in the two sequences is the same when aligned for maximum correspondence as described below. The term “complementary to” is used herein to mean all of a first sequence is complementary to at least a portion of a reference polynucleotide sequence.

The phrase “selectively (or specifically) hybridizes to” refers to the binding, duplexing, or hybridizing of a molecule only to a particular nucleotide sequence under stringent hybridization conditions when that sequence is present in a complex mixture.

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tm) for the specific sequence at a defined ionic strength pH. The Tm is the temperature (under defined ionic strength, pH, and nucleic concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For high stringency hybridization, a positive signal is at least two times background, preferably 10 times background hybridization. Those of ordinary skill will readily recognize that alternative hybridization and wash conditions can be utilized to provide conditions of similar stringency.

For PCR, a temperature of about 36° C. is typical for low stringency amplification, although annealing temperatures may vary between about 32° C. and 48° C. depending on primer length. For high stringency PCR amplification, a temperature of about 62° C. is typical, although high stringency annealing temperatures can range from about 50° C. to about 65° C., depending on the primer length and specificity. Typical cycle conditions for both high and low stringency amplifications include a denaturation phase of 90° C.-95° C. for 30 sec-2 min., an annealing phase lasting 30 sec.-2 min., and an extension phase of about 72° C. for 1-2 min.

### 2. OVERVIEW OF TRANSLOCATION CGH (tCGH)

The methods of the present can be used to detect and map chromosomal abnormalities, particularly chromosomal translocations. In one embodiment, a method of the present invention utilizes a first population of genomic nucleic acids obtained from a test sample, such as a patient sample, and a second population of genomic nucleic acids obtained from a reference sample. The reference sample may be any cells, tissues or fluid as provided herein, obtained from an individual, or any cell culture or tissue culture, that does not contain any genetic abnormality, i.e., that has a normal genetic complement of all chromosomes. The present invention employs the use of primers specific to a particular genomic locus to perform linear amplification of sequences encompassed by the genomic locus and extending into the sequence of a translocation partner to generate a probe molecule that includes both members of a translocation pair. At the same time, a reference probe is also generated using linear amplification of genomic DNA from a reference cell in the manner described for the test sample. The test and reference probes are differentially labeled, e.g., with Cy3 and Cy5, although many suitable fluorescent label pairs are known in the art. The differentially labeled probes are then hybridized to microarrays comprising genomic DNA. Generally, the sequences of the genomic DNA of the microarray are derived from a reference source such as database sequences for a particular organism, e.g., the complete database of the human, mouse, or rat genome. The pattern and extent of hybridization of the test sample probe as compared to the hybridization of a similar probe derived from a reference sample allows the identification of the translocation partner of the known genomic locus. The use of high density microarrays, such as tiling density microarrays, allows high resolution mapping of the breakpoints of the translocation.

Accordingly, if a translocation is present at a genomic locus of interest, hybridization of the test probe to a microarray comprising genomic DNA sequences from a reference cell will result in signal associated with elements corresponding to the known genomic locus as well as signals associated with elements of the microarray associated with another genomic locus. The signal associated with the other genomic locus identifies that locus as being a translocation partner of the known genomic locus. In contrast, hybridization of the microarray with the reference probe will result in hybridization exclusively associated with microarray elements corresponding with the known locus, with no hybridization signal associated with another genomic locus as observed with the test probe.

If high density tiling microarrays are used, the breakpoints of the translocation can be ascertained by determining where hybridization commences and ends in a series of microarray elements embodying contiguous segments of genomic DNA. Thus, the cessation of hybridization at a specific point along a series of elements corresponding to the known genomic locus using the test probe, with hybridization continuing along the series using the reference probe, identifies the point at which hybridization stops as being the translocation breakpoint for the known genomic locus. Similarly, the point at which hybridization by the test probe commences in a series of elements corresponding to a locus distinct from the known genomic locus, and which is negative for hybridization by the reference probe, indicates that the first element at which hybridization occurs is the breakpoint for the translocation partner of the known genomic locus.

In particular, two general embodiments of the methods of the invention are described below. Each of the embodiments of involves the performance of four linear amplification (LA) reactions:

(1) “T+”: Amplify test (e.g., tumor) DNA using the LA primer (a primer to a known sequence within a genomic locus of interest);
(2) “N+”: Amplify normal DNA using the LA primer;
(3) “T−”: Mock-amplify test (e.g., tumor) DNA (i.e. no primer present);
(4) “N−”: Mock-amplify normal DNA (i.e. no primer present).

In each embodiment (labeled Type A and Type B experiments below), the same 4 reactions are co-hybridized in different pair-wise combinations (after labeling) to two separate 2-color arrays. As described below, comparison of the two (2-color) arrays yields chromosomal rearrangement information, but the information obtained differs depending on the experiment type: both type A and B show translocation breakpoints whereas type A experiments also show genomic imbalances.

Type A Experiment:

Step 1: Co-hybridize T+ and N+samples to one array (“T+/N+array”). This array will detect both translocation breakpoints and genomic imbalances.
Step 2: Co-hybridize T− and N− samples to a second array (“T−/N− array”). This array will detect genomic imbalances but not translocation breakpoints.
Step 3: Analyze and compare results of the T+/N+ and T−/N− arrays as follows:
a) Translocations breakpoints are seen on the T+/N+ array but not on the T−/N− array. Typically, translocation breakpoints look like right triangles, with the vertical leg marking the location of the breakpoint the horizontal leg pointed away from the breakpoint.
b) Genomic imbalances are seen on both the T+/N+ and T−/N− arrays. Typically imbalances look like rectangles wherein the vertical sides mark the two ends of the duplicated or deleted genomic region.

Type B Experiment:

Step 1: Co-hybridize T+ and T− samples to one array (“T+/T− array”). The T+/T− array will detect real translocation breakpoints and “pseudo-breakpoints” but not genomic imbalances. Pseudo-breakpoints result from “non-specific” priming at multiple sites throughout the genome, presumably based on their homology to the primer sequence. Pseudo-breakpoints also are right triangular in shape.
Step 2: Co-hybridize N+ and N− samples to a second array (“N+/N− array”). The N+/N− array detects only “pseudo-breakpoints” but detects neither real translocation breakpoints nor genomic imbalances, since both N+ and N− samples start with normal DNA
Step 3: Analyze and compare results of the T+/T− and N+/N− arrays as follows:
a) Translocations breakpoints are seen on the T+/T− array but not on the N+/N− array.
b) Pseudo-breakpoints are seen on both the T+/T− AND N+/N− arrays and are ignored.

### 3. BIOLOGICAL SAMPLES

In one aspect, the methods of the present invention can be used to detect a chromosomal abnormality in a test sample. Generally, the test sample is obtained from a patient. The test sample can contain cells, tissues, or fluid obtained from a patient suspected of having a pathology or a condition associated with a chromosomal or genetic abnormality. For the purposes of diagnosis or prognosis, the pathology or condition is generally associated with genetic defects, e.g., with genomic nucleic acid base substitutions, amplifications, deletions and/or translocations. The test sample may be suspected of containing cancerous cells or nuclei from such cells. Samples may include, but are not limited to, amniotic fluid, biopsies, blood, blood cells, bone marrow, cerebrospinal fluid, fecal samples, fine needle biopsy samples, peritoneal fluid, plasma, pleural fluid, saliva, semen, serum, sputum, tears, tissue or tissue homogenates, tissue culture media, urine, and the like. Samples may also be processed, such as sectioning of tissues, fractionation, purification, or cellular organelle separation.

Methods of isolating cell, tissue, or fluid samples are well known to those of skill in the art and include, but are not limited to, aspirations, tissue sections, drawing of blood or other fluids, surgical or needle biopsies, and the like. Samples derived from a patient may include frozen sections or paraffin sections taken for histological purposes. The sample can also be derived from supernatants (of cell cultures), lysates of cells, cells from tissue culture in which it may be desirable to detect levels of mosaicisms, including chromosomal abnormalities, and copy numbers.

In one embodiment, a sample suspected of containing cancerous cells is obtained from a human patient. Samples can be derived from patients using well-known techniques such as venipuncture, lumbar puncture, fluid sample such as saliva or urine, tissue or needle biopsy, and the like. In a patient suspected of having a tumor containing cancerous cells, a sample may include a biopsy or surgical specimen of the tumor, including for example, a tumor biopsy, a fine needle aspirate, or a section from a resected tumor. A lavage specimen may be prepared from any region of interest with a saline wash, for example, cervix, bronchi, bladder, etc. A patient sample may also include exhaled air samples as taken with a breathalyzer or from a cough or sneeze. A biological sample may also be obtained from a cell or blood bank where tissue and/or blood are stored, or from an in vitro source, such as a culture of cells. Techniques for establishing a culture of cells for use as a sample source are well known to those of skill in the art.

Examples of translocations that are known to be involved with various diseases include, without limitation, t(2;5)(p23;q35)—anaplastic large cell lymphoma; t(8;14)—Burkitt's lymphoma (c-myc); t(9;22)(q34;q11)—Philadelphia chromosome, CML, ALL; t(11;14)—Mantle cell lymphoma (Bcl-1); t(11;22)(q24;q11.2-12)—Ewing's sarcoma; t(14;18)(q32;q21)—follicular lymphoma (Bcl-2); t(17;22)—dermatofibrosarcoma protuberans; t(15;17)—acute promyelocytic leukemia; t(1;12)(q21;p13)—acute myelogenous leukemia; t(9;12)(p24;p13)—CML, ALL (TEL-JAK2); t(X;18)(p11.2;q11.2)—Synovial sarcoma; t(1;11)(q42.1;q14.3)—Schizophrenia; t(12;15)(p13;q25)—(TEL-TrkC); acute myeloid leukemia, congenital fibrosarcoma, secretory breast carcinoma.

Accordingly, the present invention also provides methods of predicting, diagnosing, or providing prognoses of diseases that are caused by chromosomal rearrangements, particularly chromosomal translocations, by detecting the presence of a chromosomal translocation and determining the identity of the translocation partners. For example, if a diagnosis of Burkitt's lymphoma is desired, a primer for linear amplification of an appropriate immunoglobulin regulatory locus would be used to generate a probe for hybridization to a human microarray. Using the methods of the invention, a diagnosis of Burkitt's lymphoma would be indicated if the translocation partner for the immunoglobulin locus is identified as the gene for MYC. In one embodiment, the methods of the invention are particularly well suited for the diagnosis or prognosis of a cancer associated with a balanced chromosomal translocation.

The term “cancer” refers to human cancers and carcinomas, leukemias, sarcomas, adenocarcinomas, lymphomas, solid and lymphoid cancers, etc. Examples of different types of cancer include, but are not limited to, monocytic leukemia, myelogenous leukemia, acute lymphocytic leukemia, and acute myelocytic leukemia, chronic myelocytic leukemia, promyelocytic leukemia, breast cancer, gastric cancer, bladder cancer, ovarian cancer, thyroid cancer, lung cancer, prostate cancer, uterine cancer, testicular cancer, neuroblastoma, squamous cell carcinoma of the head, neck, cervix and vagina, multiple myeloma, soft tissue and osteogenic sarcoma, colorectal cancer, liver cancer (i.e., hepatocarcinoma), renal cancer (i.e., renal cell carcinoma), pleural cancer, pancreatic cancer, cervical cancer, anal cancer, bile duct cancer, gastrointestinal carcinoid tumors, esophageal cancer, gall bladder cancer, small intestine cancer, cancer of the central nervous system, skin cancer, choriocarcinoma; osteogenic sarcoma, fibrosarcoma, glioma, melanoma, B-cell lymphoma, non-Hodgkin's lymphoma, Burkitt's lymphoma, Small Cell lymphoma, Large Cell lymphoma, and the like.

In another embodiment, the methods of the invention can be used to detect a chromosomal or genetic abnormality in a fetus. For example, prenatal diagnosis of a fetus may be indicated for women at increased risk of carrying a fetus with chromosomal or genetic abnormalities. Risk factors are well known in the art, and include, for example, advanced maternal age, abnormal maternal serum markers in prenatal screening, chromosomal abnormalities in a previous child, a previous child with physical anomalies and unknown chromosomal status, parental chromosomal abnormality, and recurrent spontaneous abortions.

The invention methods can be used to perform prenatal diagnosis using any type of embryonic or fetal cell. Fetal cells can be obtained through the pregnant female, or from a sample of an embryo. Thus, fetal cells are present in amniotic fluid obtained by amniocentesis, chorionic villi aspirated by syringe, percutaneous umbilical blood, a fetal skin biopsy, a blastomere from a four-cell to eight-cell stage embryo (pre-implantation), or a trophectoderm sample from a blastocyst (pre-implantation or by uterine lavage). Body fluids with sufficient amounts of genomic nucleic acid also may be used.

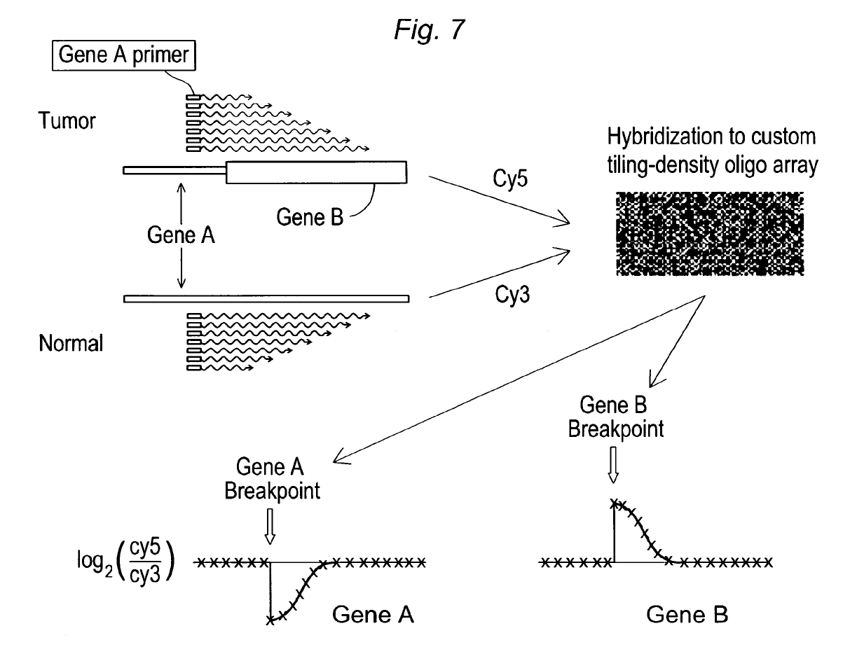

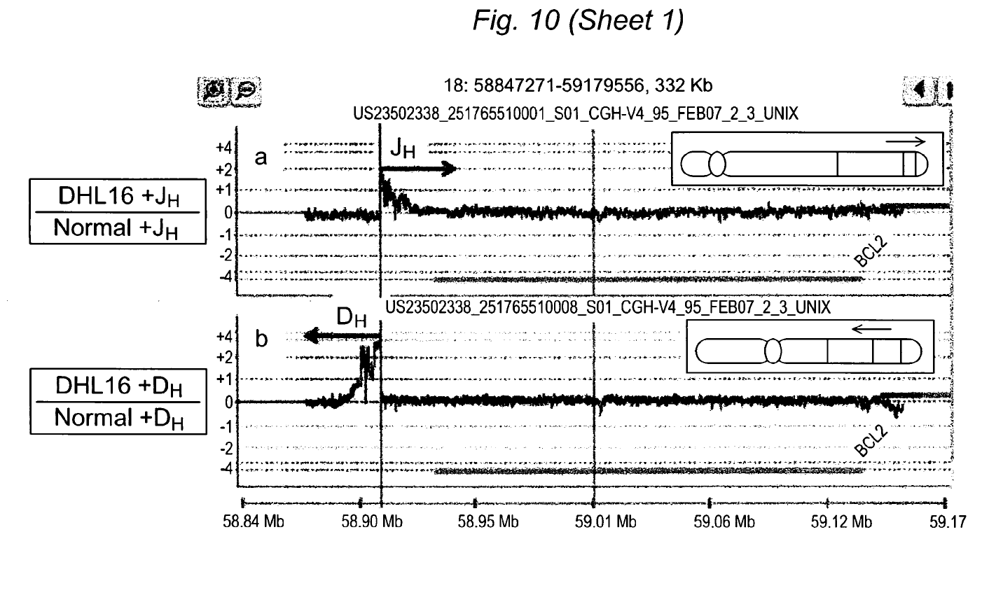

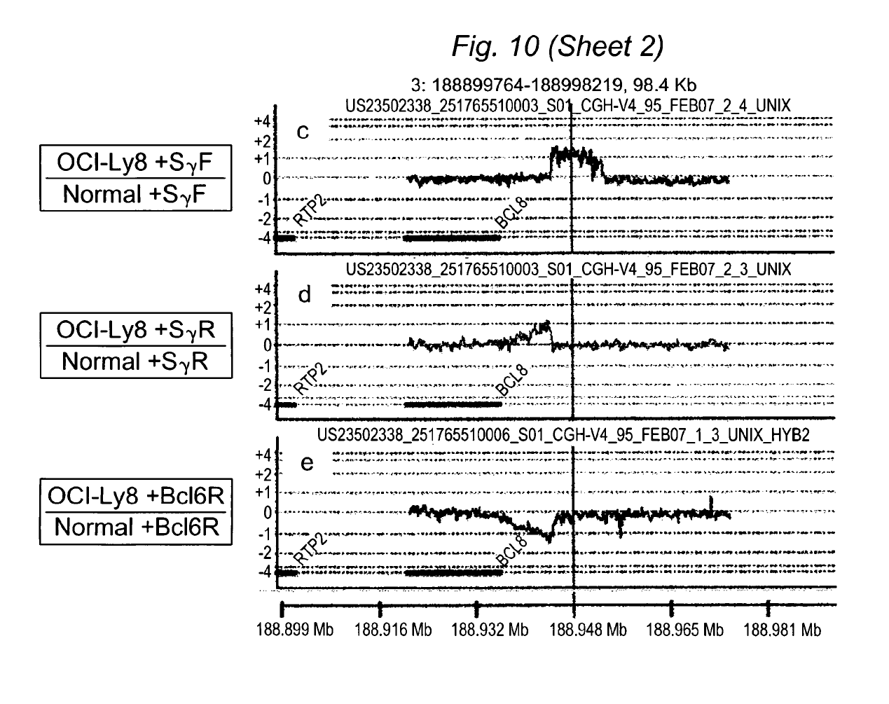

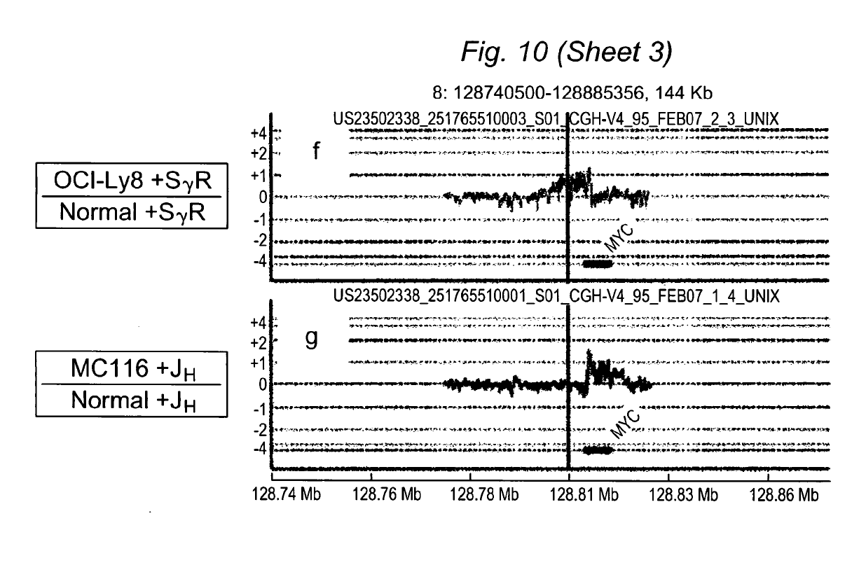

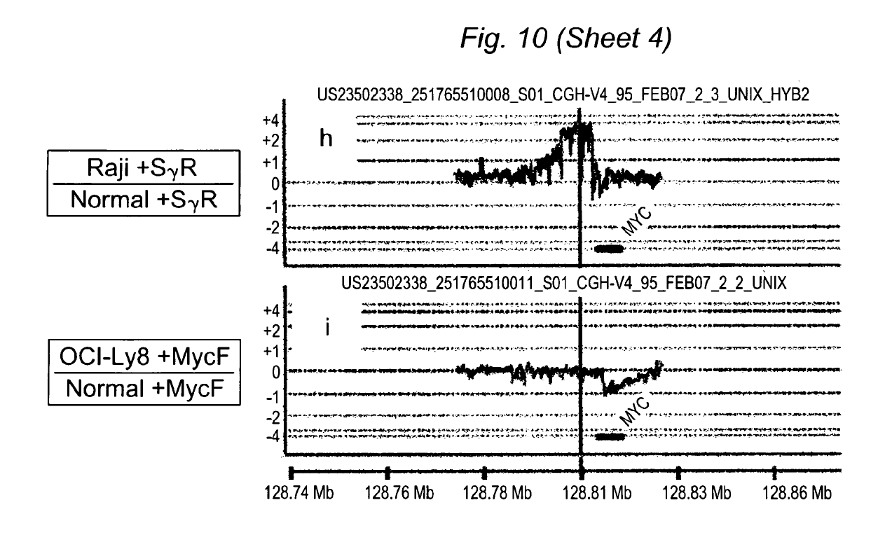

In one embodiment, the tCGH methods of the invention comprise the detection and mapping of breakpoints in both partner genes involved in a chromosomal translocation (see, for example, genes A and B in FIG. 7). When detecting both partner genes of a translocation, the amplicon produced by the linear amplification of gene A, e.g. the gene targeted by the primer, will result in an ‘inverted’ right triangular pattern of hybridization (see, for example, FIGS. 10e and 10i, which show an inverted hybridization pattern for the BCL6 and MYC amplicons, respectively).

In a second embodiment, the present invention provides methods of tCGH analysis which comprise multiplex linear amplification for the detection of chromosomal rearrangements at more than one locus simultaneously. In one embodiment, the multiplex amplification is performed using a mixture of linear amplification primers. In one example, a mixture of seven D_{H} primers (see, Table 3) may be used to cover multiple D_{H} rearrangements (van Dongen, Langerak et al., Leukemia 17(12): 2257-317 (2003)). In another embodiment, a mixture of 5 primers for the linear amplification of loci associated with myeloid leukemia (MPM mix, see, Table 5) may be used to amplify three different myeloid leukemia translocations: (1) BCR-ABL fusion=t(9;22) in CML (chronic myelogenous leukemia), (2) PML-RARA fusion=t(15;17) in acute promyelocytic leukemia, and (3) acute myeloid leukemia (AML) with inv(16)/t(16;16). In yet other embodiments, an 821 primer mix or P1/P7 primer mix (Table 5) may be used for multiplex linear amplification in conjunction with tCGH analysis of balanced translocations.

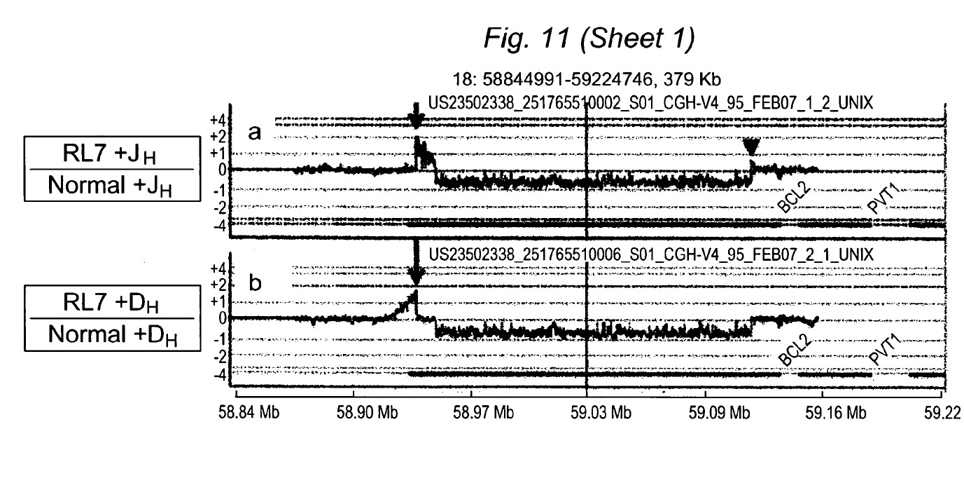

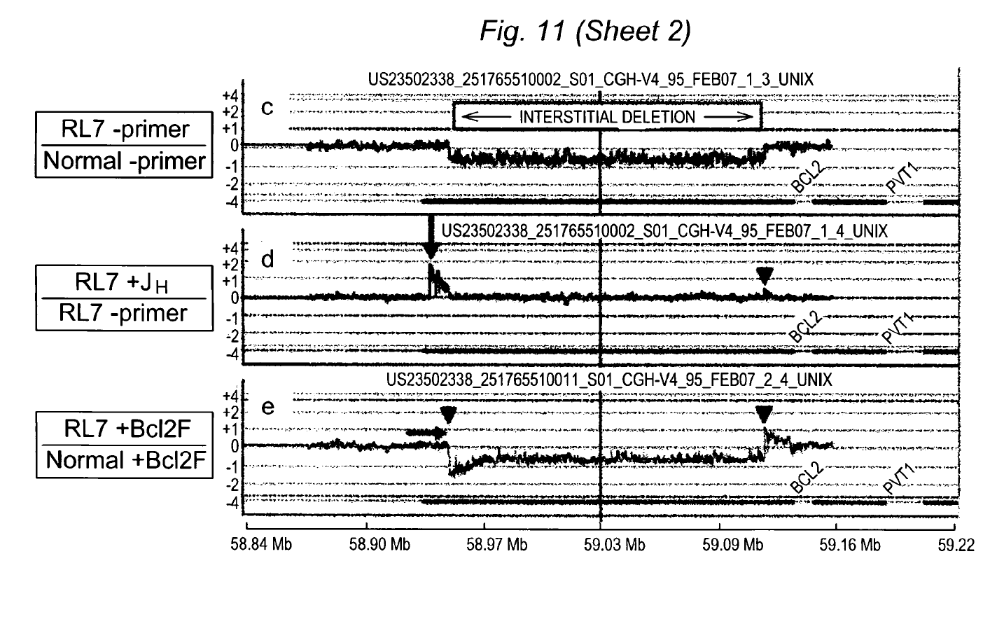

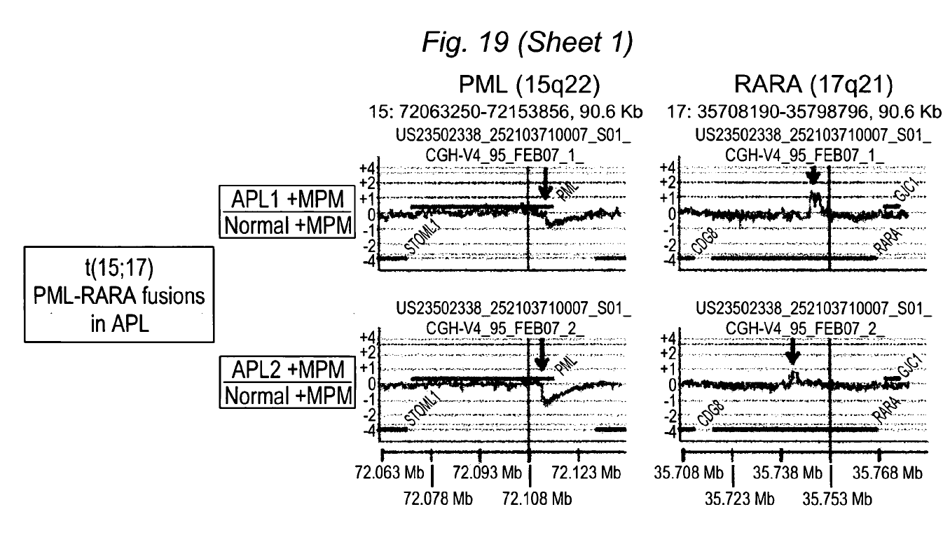

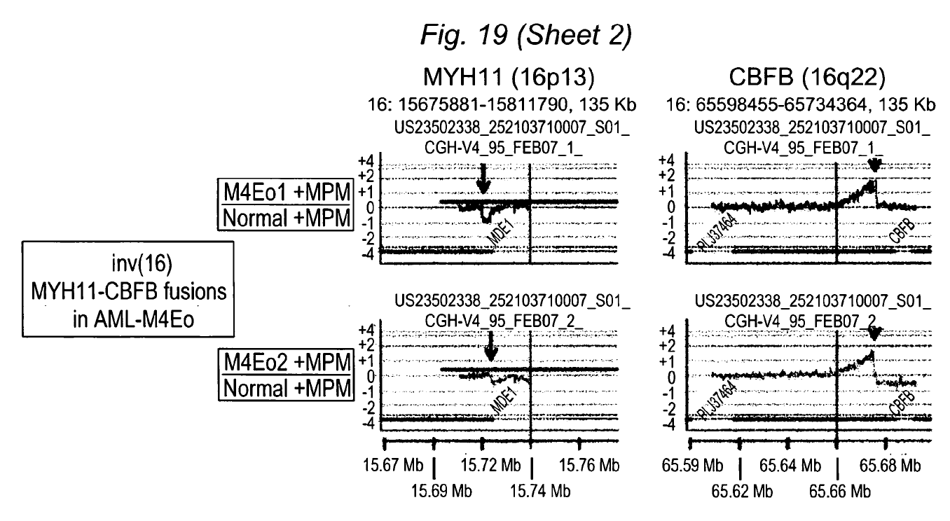

In a third embodiment, the methods provided by the present invention may comprise the detection of a chromosomal rearrangement other than a balanced translocation. In certain embodiments, this chromosomal rearrangement may comprise a deletion, a duplication, an amplification, an inversion, or an unbalanced translocation. For example, FIG. 11e shows the detection, by tCGH analysis, of an intronic interstitial BCL2 deletion by amplification across the deletion breakpoints. Similarly, FIG. 19 shows the detection of a chromosomal inversion fusing the MYH11 and CBFB genes inv(16) (bottom panel), by tCGH analysis.

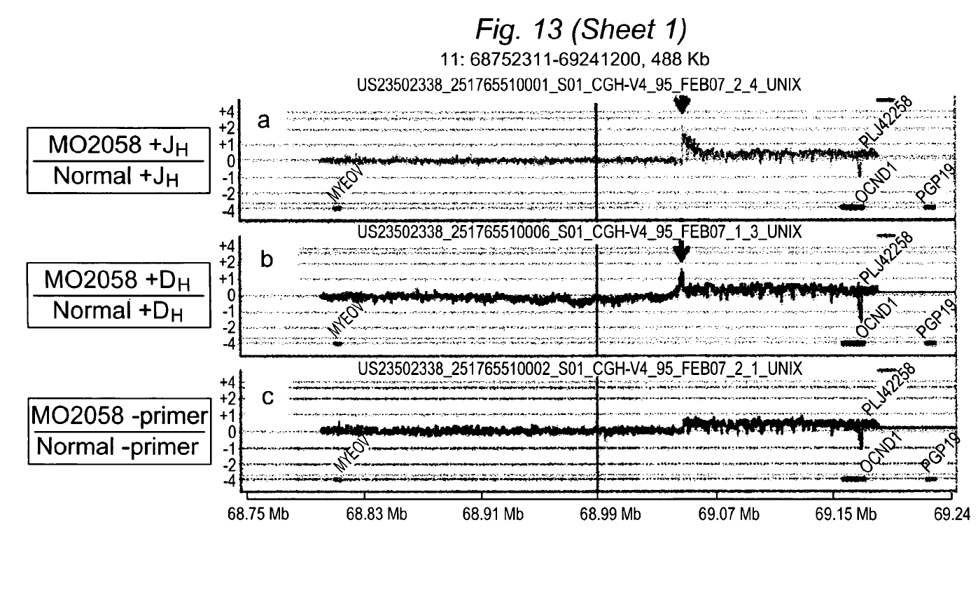

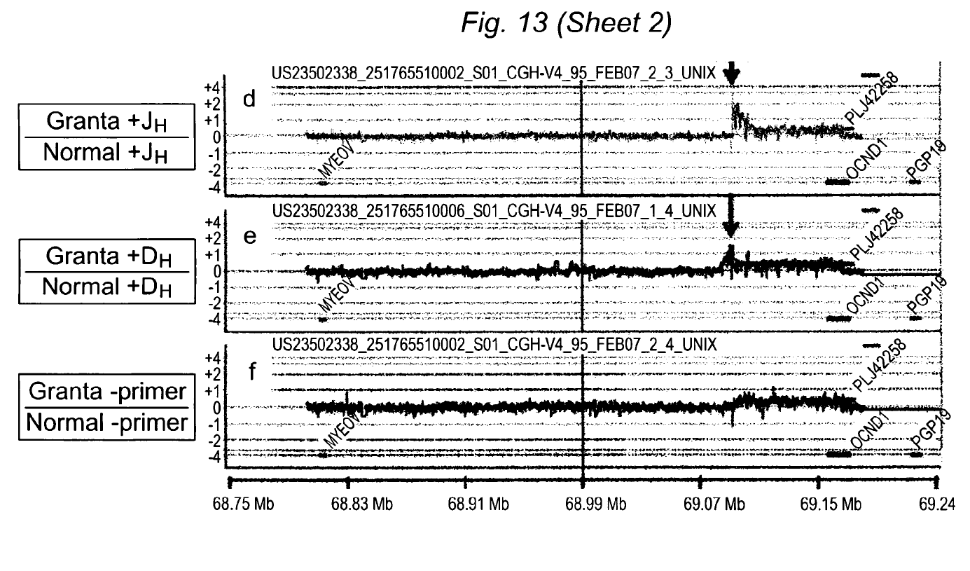

In a fourth embodiment, the present invention may comprise the simultaneous detection of both balanced rearrangements and imbalanced chromosomal abnormalities. In certain embodiments, the methods of the invention allow for simultaneous detection when the breakpoint for the imbalance is coincident with that of the balanced rearrangement. For example, FIG. 13 shows the simultaneous detection of a balanced translocation and chromosomal duplication in both the MO2058 and Granta 519 cell lines, at the IgH-CCND1 translocation breakpoint.

In a fifth embodiment, the present invention provides a method of diagnosing or providing a prognosis for a disease in an individual by detecting a chromosomal rearrangement. In one embodiment, the invention provides a method of diagnosing a lymphoma in an individual, the method comprising the detection of a novel breakpoint selected from those found in Table 2 in a sample from said individual. In certain embodiments, the method comprises the detection of a disease selected from a B cell lymphoma, a Mantle Cell Lymphoma (MCL), a myeloma, a Diffuse Large B-Cell Lymphoma (DLBCL), Burkitt's lymphoma, a B-Cell Lymphoma, and a Follicle Center Lymphoma (FCL). In certain embodiments, the detection is by PCR analysis, sequencing, mass spectrometry, hybridization, or tCGH analysis. Suitable primers for PCR analysis or sequencing of a novel translocation listed in Table 2 include, without limitation, SEQ ID NOS:27, 28, 30, 31, 33, 34, 36, 37, 39, 40, 42, 43, 45, 46, 48, 49, 51, 52, 54, 55, 57, 58, 60, 61, 63, 64, and functional equivalents thereof.

In one specific embodiment, the invention provides a method of diagnosing or providing a prognosis for B cell lymphoma or Mantle Cell Lymphoma (MCL) in an individual by detecting, in a biological sample from the individual, an I_{g}H-CCND1 translocation, wherein the CCND1 breakpoint is selected from the group consisting of chr11:69,055,996, chr11:69,100,509, chr11:69,131,130, chr11:69,056,460, 68,989,831, chr11:69,082,854, chr11:69,059,199, and chr18:58,944,421. In a second specific embodiment, the invention provides a method of diagnosing or providing a prognosis for myeloma in an individual by detecting, in a biological sample from the individual, an I_{g}H-CCND1 translocation, wherein the CCND1 breakpoint is chr11:69,153,045 or chr11:69,153,019. In a third specific embodiment, the invention provides a method of diagnosing or providing a prognosis for DLBCL in an individual by detecting, in a biological sample from the individual, an I_{g}H-BCL2 translocation, wherein the BCL2 breakpoint is selected from chr18:58,944,489, chr18:58,914,890, chr18:58,944,475, and chr18:58,938,252. In a fourth specific embodiment, the invention provides a method of diagnosing or providing a prognosis for DLBCL in an individual by detecting, in a biological sample from the individual, an I_{g}H-BCL6 translocation, wherein the BCL2 breakpoint is chr3:188,945,670 or chr3:188,945,699. In a fifth specific embodiment, the invention provides a method of diagnosing or providing a prognosis for a B-cell Lymphoma in an individual by detecting, in a biological sample from the individual, an I_{g}H-MYC translocation, wherein the MYC breakpoint is chr8:128,818,596, chr8:128,817,581, or chr8:128,816,104.

### 4. GENERATION OF PROBES FOR DETECTING BALANCED TRANSLOCATIONS

For the detection of translocations, any method that results in the linear amplification of a DNA that spans a potential site of translocation may be used. Examples of linear amplification methods that may be used in the practice of the invention include PCR amplification using a single primer. See, Liu, C. L., S. L. Schreiber, et al., BMC Genomics, 4: Art. No. 19, May 9, 2003.

An exemplary set of conditions for linear amplification include reactions in a 50 μl volume containing 1 μg genomic DNA, 200 mM dNTPs, and 150 nM linear amplification primer. The amplification can be performed using the Advantage 2 PCR Enzyme System (Clontech) as follows: denaturation at 95° C. for 5 min followed by 12 cycles of (95° C./15 sec, 60° C./15 sec, and 68° C./6 min).

Probes may be labeled during the course of linear amplification or after amplification has occurred. In the specific examples outlined below, labels are incorporated in a separate step after the linear amplification by oligonucleotide (random hexamers) mediated primer extension with a DNA polymerase. With this protocol, both the original genomic DNA samples and the linear amplification products will give rise to labeled probes that generate signals. After hybridization, the resulting data will yield information on both chromosomal aberrations from differential genomic DNA signals as seen with normal aCGH, but also reveal chromosomal rearrangements coming from differential signals arising from the linear amplification products. If labels are incorporated simply in the linear amplification products, as would happen if the labeled dNTPs were included in the linear amplification step, then only translocations would be revealed and not chromosomal abnormalities like amplifications and deletions. Useful labels include, e.g., fluorescent dyes (e.g., Cy5, Cy3, FITC, rhodamine, lanthamide phosphors, Texas red), ³²P, ³⁵S, ³H, ¹⁴C, ¹²⁵I, ¹³¹I, electron-dense reagents (e.g., gold), enzymes, e.g., as commonly used in an ELISA (e.g., horseradish peroxidase, beta-galactosidase, luciferase, alkaline phosphatase), colorimetric labels (e.g., colloidal gold), magnetic labels (e.g., Dynabeads), biotin, dioxigenin, or haptens and proteins for which antisera or monoclonal antibodies are available. The label can be directly incorporated into the nucleic acid to be detected, or it can be attached to a probe (e.g., an oligonucleotide) or antibody that hybridizes or binds to the nucleic acid to be detected. The detectable label can be incorporated into, associated with or conjugated to a nucleic acid. The association between the nucleic acid and the detectable label can be covalent or non-covalent. Label can be attached by spacer arms of various lengths to reduce potential steric hindrance or impact on other useful or desired properties.

### 5. MICROARRAYS

Any known microarray and/or method of making and using microarrays can be used in the practice of the present invention, such as those disclosed, for example, in U.S. Pat. Nos. 6,277,628; 6,277,489; 6,261,776; 6,258,606; 6,054,270; 6,048,695; 6,045,996; 6,022,963; 6,013,440; 5,965,452; 5,959,098; 5,856,174; 5,830,645; 5,770,456; 5,632,957; 5,556,752; 5,143,854; 5,807,522; 5,800,992; 5,744,305; 5,700,637; 5,556,752; 5,434,049; see also, e.g., WO 99/51773; WO 99/09217; WO 97/46313; WO 96/17958; see also, e.g., Johnston, Curr. Biol. 8:R171-R174, 1998; Schummer, Biotechniques 23:1087-1092, 1997; Kern, Biotechniques 23:120-124, 1997; Solinas-Toldo, Genes, Chromosomes & Cancer 20:399-407, 1997; Bowtell, Nature Genetics Supp. 21:25-32, 1999. See also published U.S. patent applications Ser. Nos. 20010018642; 20010019827; 20010016322; 20010014449; 20010014448; 20010012537; 20010008765.

The tCGH methods of the invention can be performed using a variety of commercially available CGH arrays, as well as custom designed arrays, that can be commercially fabricated. Examples of commercially available high density arrays and kits include those available from Agilent Technologies for human, mouse, and rat genomic analysis (e.g., G4411B and G4412A), custom tiling arrays manufactured by Nimblegen (Roche) and whole-genome tiling arrays made by Affymetrix.

In one embodiment, the present invention provides a novel high density array for the detection of a balanced translocation associated with leukemia. In certain embodiments, the high density arrays of the present invention are useful for the diagnosis, for providing a prognosis, or for genotyping a leukemia, such as a myeloid leukemia or a lymphoma. In a particular embodiment, the invention provides an array for detecting the loci found in Table 1, Table 2, and/or Table 4. In certain embodiments, an array of the invention allows for the detection of the novel breakpoints found in Table 2. In certain embodiments, the arrays of the invention allow for the detection of both partner genes in a translocation, for tCGH analysis. In a particular embodiment, the present invention provides an AML high density array as outlined in Table 4.

In yet another embodiment, the present invention provides primer mixtures that are useful for the detection of balanced translocations associated with a disease, such as cancer. In a particular embodiment, the cancer is a leukemia or a myeloid leukemia. In certain embodiments, the primer mixes provided by the invention are useful for the linear amplification of genomic loci that are commonly involved in balanced translocations in individuals suffering from a disease. In some embodiments, the primer mixes of the invention are useful for multiplex linear amplification and multiplex tCGH analysis. In specific embodiments of the invention, the primer mixes are selected from a myeloid primer mix (MPM), an 821 mix, and a P1/P7 mix.

The resolution of array-based CGH is primarily dependent upon the number, size and map positions of the nucleic acid elements within the array, which are capable of spanning the entire genome. In a particularly advantageous embodiment of the present invention, oligonucleotide nucleic acid elements are used to form microarrays at tiling density. See, e.g., Mockler, T. C. and J. R. Ecker, Genomics 85: 1 (2005); Bertone, P., M. Gerstein, et al., Chromosome Research, 13: 259 (2005).

### 6. HYBRIDIZATION OF MICROARRAYS

Any of a number of previously described methods for carrying out comparative genomic hybridization may be used in the practice of the present invention, such as those described in U.S. Pat. Nos. 6,197,501; 6,159,685; 5,976,790; 5,965,362; 5,856,097; 5,830,645; 5,721,098; 5,665,549; 5,635,351; Diago, Am. J. Pathol. 158:1623-1631, 2001; Theillet, Bull. Cancer 88:261-268, 2001; Werner, Pharmacogenomics 2:25-36, 2001; Jain, Pharmacogenomics 1:289-307, 2000.

In some cases, prior to the hybridization of a specific probe of interest, it is desirable to block repetitive sequences. A number of methods for removing and/or blocking hybridization to repetitive sequences are known (see, e.g., WO 93/18186). As an example, it may be desirable to block hybridization to highly repeated sequences such as Alu sequences. One method to accomplish this exploits the fact that hybridization rate of complementary sequences increases as their concentration increases. Thus, repetitive sequences, which are generally present at high concentration will become double stranded more rapidly than others following denaturation and incubation under hybridization conditions. The double stranded nucleic acids are then removed and the remainder used in hybridizations. Methods of separating single from double stranded sequences include using hydroxyapatite or immobilized complementary nucleic acids attached to a solid support, and the like. Alternatively, the partially hybridized mixture can be used and the double stranded sequences will be unable to hybridize to the target.

Also, unlabeled sequences which are complementary to the sequences sought to be blocked can be added to the hybridization mixture. This method can be used to inhibit hybridization of repetitive sequences as well as other sequences. For example, Cot-1 DNA can be used to selectively inhibit hybridization of repetitive sequences in a sample. To prepare Cot-1 DNA, DNA is extracted, sheared, denatured and renatured. Because highly repetitive sequences reanneal more quickly, the resulting hybrids are highly enriched for these sequences. The remaining single stranded DNA (i.e., single copy sequences) is digested with S1 nuclease and the double stranded Cot-1 DNA is purified and used to block hybridization of repetitive sequences in a sample. Although Cot-1 DNA can be prepared as described above, it is also commercially available (BRL).

Hybridization conditions for nucleic acids in the methods of the present invention are well known in the art. Hybridization conditions may be high, moderate or low stringency conditions. Ideally, nucleic acids will hybridize only to complementary nucleic acids and will not hybridize to other non-complementary nucleic acids in the sample. The hybridization conditions can be varied to alter the degree of stringency in the hybridization and reduce background signals as is known in the art. For example, if the hybridization conditions are high stringency conditions, a nucleic acid will bind only to nucleic acid target sequences with a very high degree of complementarity. Low stringency hybridization conditions will allow for hybridization of sequences with some degree of sequence divergence. The hybridization conditions will vary depending on the biological sample, and the type and sequence of nucleic acids. One skilled in the art will know how to optimize the hybridization conditions to practice the methods of the present invention.

An exemplary hybridization conditions is as follows. High stringency generally refers to conditions that permit hybridization of only those nucleic acid sequences that form stable hybrids in 0.018M NaCl at 65° C. High stringency conditions can be provided, for example, by hybridization in 50% formamide, 5×Denhardt's solution, 5×SSC (saline sodium citrate) 0.2% SDS (sodium dodecyl sulphate) at 42° C., followed by washing in 0.1×SSC, and 0.1% SDS at 65° C. Moderate stringency refers to conditions equivalent to hybridization in 50% formamide, 5×Denhardt's solution, 5×SSC, 0.2% SDS at 42° C., followed by washing in 0.2×SSC, 0.2% SDS, at 65° C. Low stringency refers to conditions equivalent to hybridization in 10% formamide, 5×Denhardt's solution, 6×SSC, 0.2% SDS, followed by washing in 1×SSC, 0.2% SDS, at 50° C.

Reading and Interpretation of tCGH Assays

The identification of translocation partners of known genetic loci and the determination of translocation breakpoints is based on a determination of the pattern and intensity of hybridization of labeled probes to one or more nucleic acid elements of the microarray. Typically, the position of a hybridization signal on an array, the hybridization signal intensity, and the ratio of intensities, produced by detectable labels associated with a sample or test probe and a reference probe is determined. The determination of an element that hybridizes to the sample or test probe, but not to the reference probe, identifies the sequence contained within that element as a translocation partner of the known genetic locus. Identical hybridization patterns between the test probe and the reference probe indicate that the tested sample does not contain a translocation at the known genetic locus. When tiling density microarrays are used, the translocation breakpoints can be determined by ascertaining where in a series of microarray elements representing contiguous genomic segments, hybridization commences or ends. Thus, in the case of a balanced translocation, hybridization will begin at a particular DNA sequence within a gene distinct from the known genomic locus. The sequence embodied by the first element in a contiguous sequence of the distinct gene identifies that sequence as representing the breakpoint within the second gene. Conversely, with respect to the known genomic locus, the element within a contiguous sequence where hybridization ends marks that element as representing the translocation breakpoint within the known genomic locus.

Moreover, typically, the greater the ratio of the signal intensities on a target nucleic acid segment, the greater the copy number ratio of sequences in the two samples that bind to that element. Thus comparison of the signal intensity ratios among target nucleic acid segments permits comparison of copy number ratios of different sequences in the genomic nucleic acids of the two samples.

In general, any apparatus or method that can be used to detect measurable labels associated with nucleic acids that bind to an array-immobilized nucleic acid segment may be used in the practice of the invention. Devices and methods for the detection of multiple fluorophores are well known in the art, see, e.g., U.S. Pat. Nos. 5,539,517; 6,049,380; 6,054,279; 6,055,325; and 6,294,331. Any known device or method, or variation thereof, can be used or adapted to practice the methods of the invention, including array reading or “scanning” devices, such as scanning and analyzing multicolor fluorescence images; see, e.g., U.S. Pat. Nos. 6,294,331; 6,261,776; 6,252,664; 6,191,425; 6,143,495; 6,140,044; 6,066,459; 5,943,129; 5,922,617; 5,880,473; 5,846,708; 5,790,727; and, the patents cited in the discussion of arrays, herein. See also published U.S. Patent Application Ser. Nos. 20010018514; 20010007747; and published international patent applications Nos. WO0146467 A; WO9960163 A; WO0009650 A; WO0026412 A; WO0042222 A; WO0047600 A; and WO0101144 A.

### 7. KITS OF THE INVENTION

The present invention also provides kits to facilitate and/or standardize the tCGH methods provided herein. Materials and reagents for executing the various methods of the invention can be provided in kits to facilitate these methods. As used herein, the term “kit” refers to a combination of articles that facilitate a process, assay, analysis, diagnosis, prognosis, or manipulation.

In one embodiment, the kits provided by the present invention may comprise a nucleic acid primer for the linear amplification of a genomic locus implicated in balanced translocation. In certain embodiments, the kits may comprise a primer mix for the multiplex linear amplification of multiple genomic loci. In other embodiments, the kits of the invention may comprise a high density tiling array for use in tCGH analysis of balanced chromosomal translocations. In certain embodiments, the present invention provides kits useful for the diagnosis, or prognosis of a disease characterized by a balanced translocation. In particular embodiments, the disease is a cancer, such as a lymphoma or a leukemia.

In a particular embodiment, the present invention provides a kit comprising a high density tiling array for the detection of a balanced translocation associated with a myeloid leukemia. A kit of the invention may further comprise a primer mix for the multiplex linear amplification of genomic loci involved in balanced translocations associated with a myeloid leukemia. In a specific embodiment, the tiling array may be an AML pilot array and a primer mix may be selected from the MPM mix, the 821 mix, or the P1/P7 mix.

The following examples are offered to illustrate, but not to limit the claimed invention.

### EXAMPLES

### Example 1

### Identification of J_{H}-Associated Translocation Breakpoints

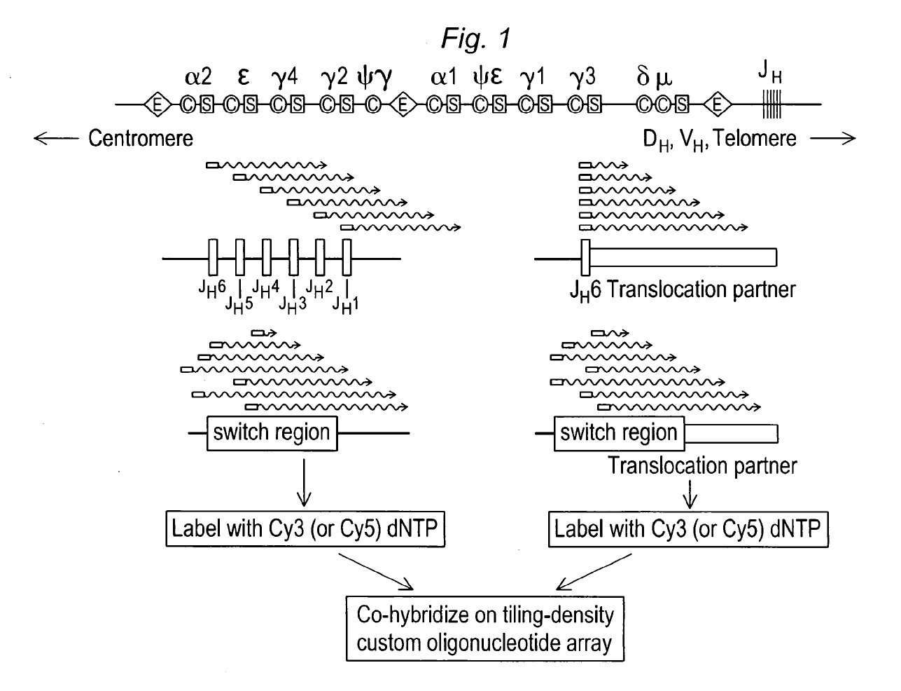

ArrayCGH is designed to detect genomic imbalances but not balanced genomic rearrangements. We thus sought a means for creating synthetic genomic imbalances to mark the sites of balanced translocations on standard CGH arrays. Using balanced immunoglobulin translocations in lymphoma cell lines as a model system, we developed an enzymatic linear amplification reaction that renders balanced translocations detectable by array CGH simply by modifying genomic DNA in a targeted linear amplification step prior to fluorescent labeling and microarray hybridization. As outlined in FIG. 1, J_{H}-associated translocation breakpoints are enzymatically amplified using a J_{H} consensus primer, (van Dongen, 2003) resulting in linear amplification across the breakpoint junction into the translocation partner locus. Use of a single primer enables amplification of J_{H}-associated translocations regardless of the identity of the IgH partner gene.

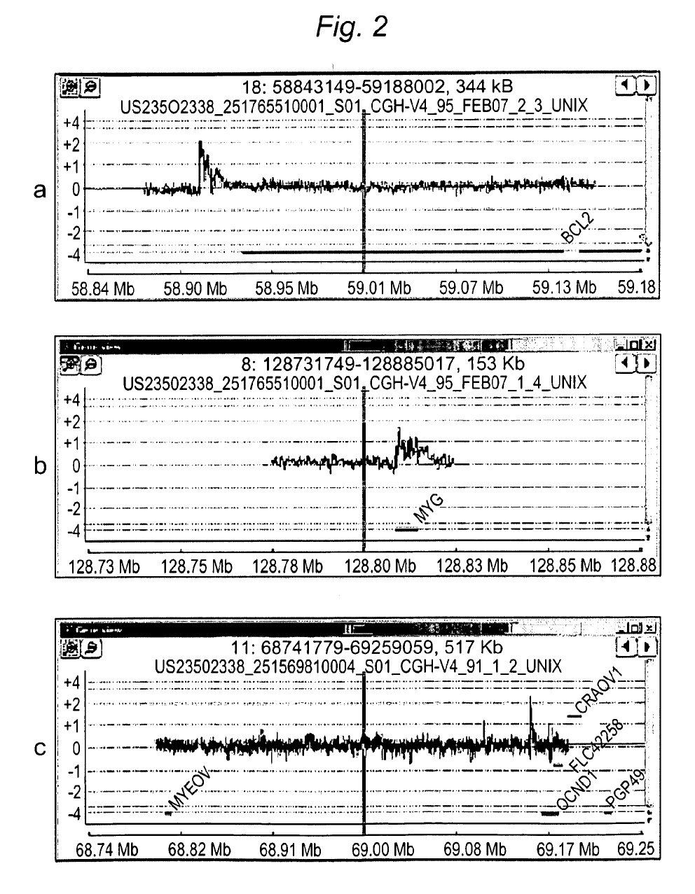

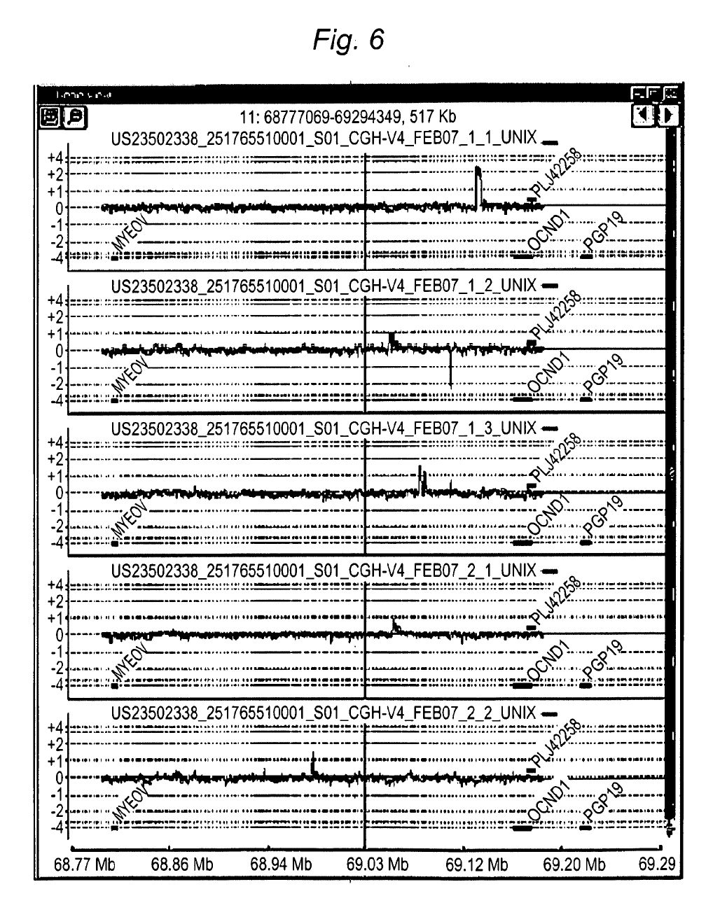

In a typical tCGH experiment, genomic DNA from a lymphoma and from a normal control undergoes linear amplification and fluorescent labeling with Cy3 (lymphoma) or Cy5 (control), and is then combined and hybridized to a custom oligonucleotide array representing common IgH fusion partner loci at tiling-density (FIG. 1). J_{H}-associated translocations involving the BCL2 and MYC loci are illustrated in FIGS. 2a and 2b. The breakpoints are identified on tCGH arrays by virtue of their characteristic right-triangular shape with a vertical leg marking the genomic location of the breakpoint in the non-IgH locus. The height of the vertical leg indicates the extent of J_{H}-associated linear amplification. This triangular shape is consistent with a linear amplification step that produces DNA fragments of varying size and with gradual decay of the amplification intensity with increasing distance from the J_{H} primer. The shape of this breakpoint profile depends on the amplification conditions, with increasing extension time resulting in a wider profile on tCGH arrays (FIG. 6).

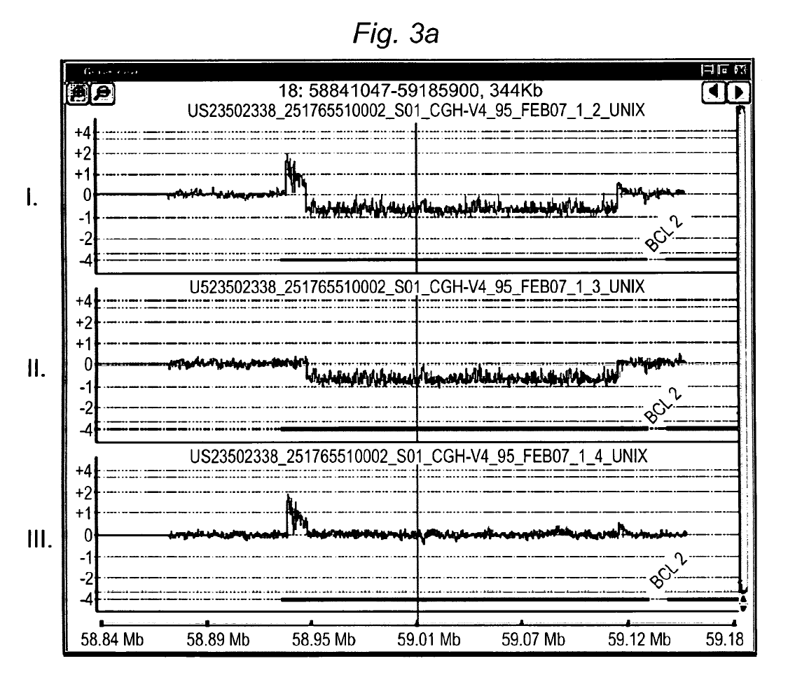

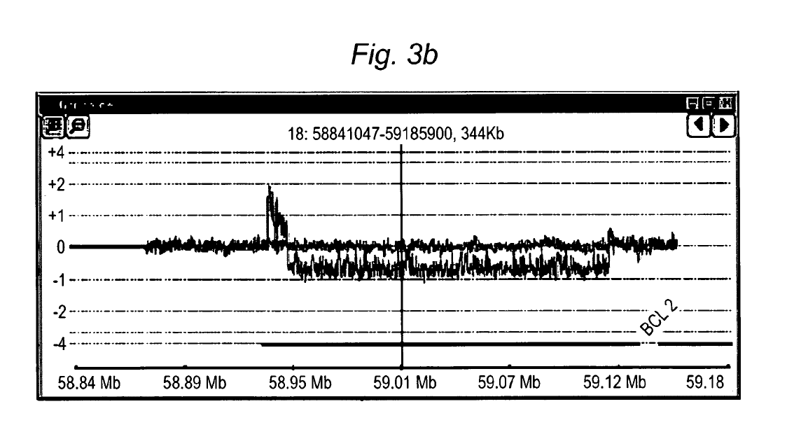

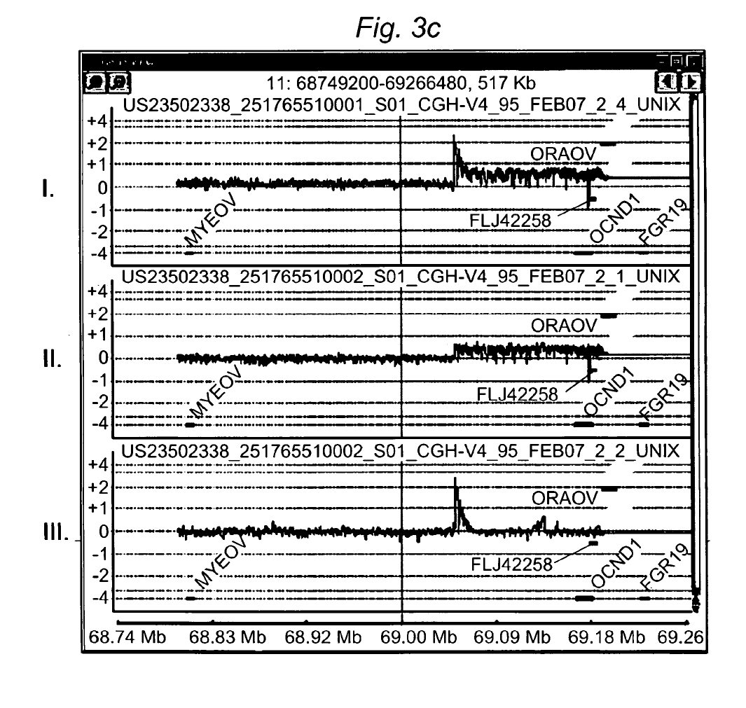

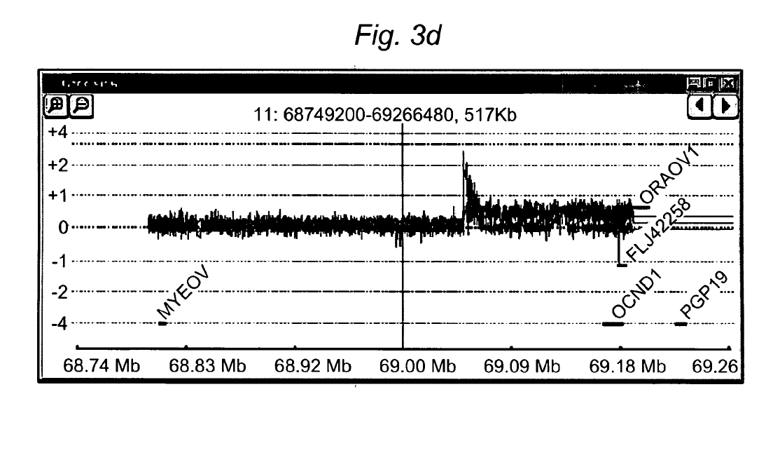

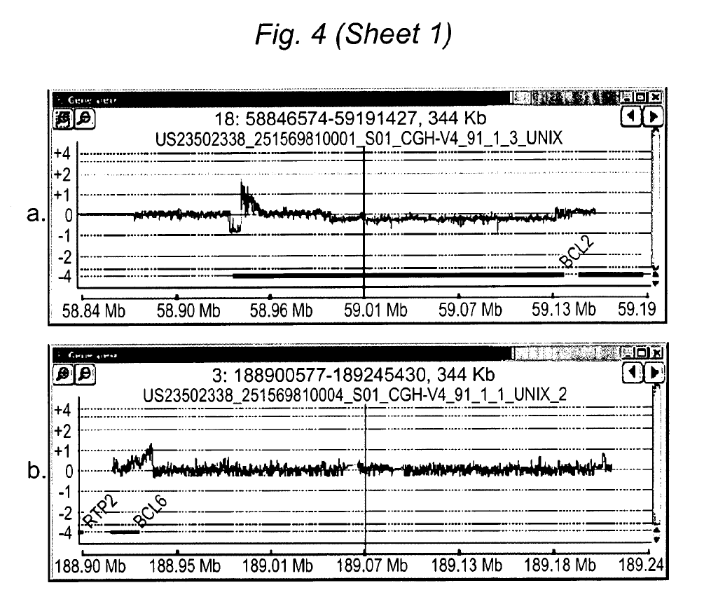

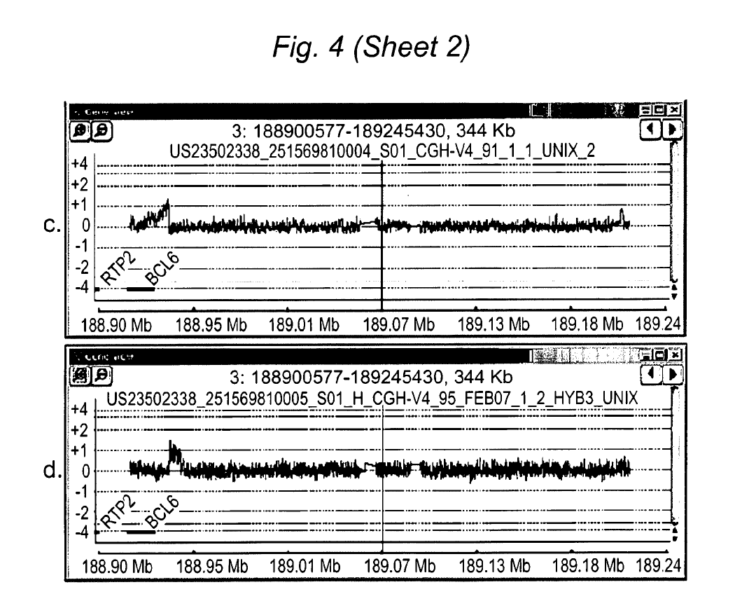

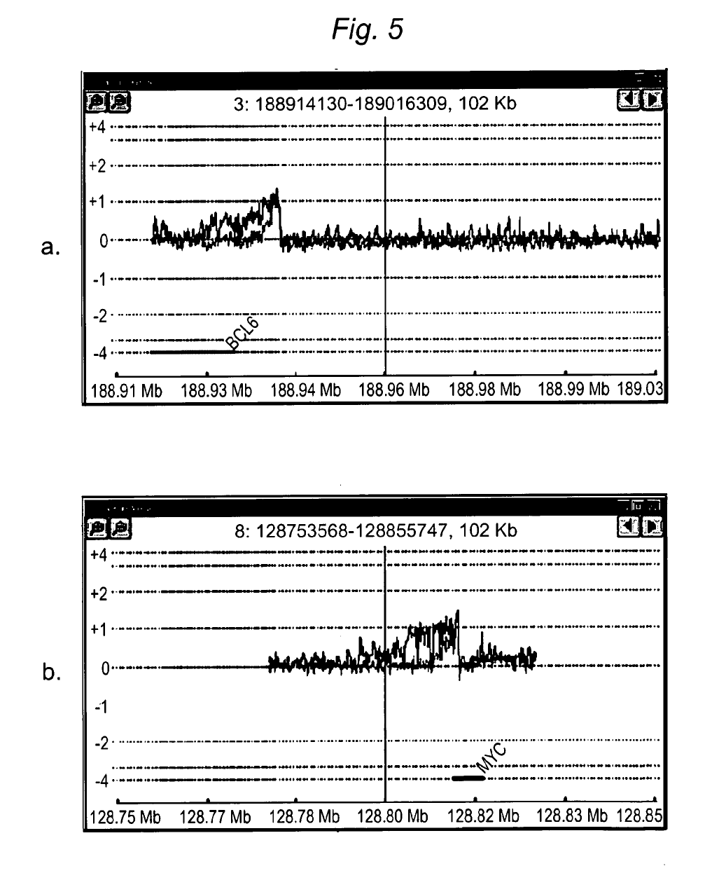

The use of normal genomic DNA as a hybridization control allows both balanced translocations and chromosomal imbalances to be detected on the same array. The RL7 (Lipford, 1987) (FIG. 3a, top panel) and OCI-Ly8 (Tweeddale, 1987) cell lines (FIG. 4a), for example, were both found to have J_{H}-BCL2 translocations and large deletions in BCL2 intron 2. A small deletion adjacent to the J_{H}-BCL2 breakpoint in OCI-Ly8 is likely to have arisen in association with that translocation. In the MO2058 mantle cell lymphoma (MCL) line (Meeker, 1991) (FIG. 3c), there is duplication of CCND1 locus telomeric to the J_{H} breakpoint and a small deletion within the CCND1 3′UTR. The asymmetric shape of the array profile associated with balanced translocations generally allow these to be readily distinguished from genomic imbalances on tCGH arrays. This is illustrated by comparison of the tCGH results to mock-amplification experiments in which only genomic imbalances are identified, as in typical arrayCGH (FIGS. 3a and 3c, middle panels). tCGH also can be used to identify balanced translocations in isolation (without detecting genomic imbalances) by using mock-amplified test DNA instead of linearly-amplified normal DNA as the control specimen (FIGS. 3a and 3c, bottom panel). tCGH experiments performed in this manner exhibit “pseudo-breakpoints” at multiple locations (supplementary data, not shown), an artifact that is minimized by used of amplified normal DNA in typical tCGH experiments.

### Example 2

### Identification of IgH Switch (S_{H})-Associated Translocation Breakpoints

Linear amplification primers were then designed to identify translocations involving the IgH switch (S_{H}) regions. Human S_{H} regions contain multiple tandem repeats of a characteristic repetitive sequence unit: in the S_{μ}, S_{α}, and S_{ε} E regions, the repeat unit is the degenerate pentameric sequence G(A/G)GCT whereas in the S_{γ} regions it is 80-90 nt long and more complex (Max, 1982; Mills, 1990; Mills, 1995). S_{H}-associated translocation breakpoints are distributed throughout these repetitive regions. To facilitate detection of such diverse breakpoints, linear amplification primers were designed to recognize these repeat units and prime synthesis at multiple locations within the S_{μ}/S_{α}/S_{ε} regions (S₅ primer) or the S_{γ} repeat regions (S_{γ} primer). tCGH performed using the S₅ primer in place of the J_{H} was used to identify a S_{μ}-MYC translocation in the Burkitt lymphoma cell line Raji (Dyson, 1985) (not shown) and a cryptic S_{α}-CCND1 fusion in the multiple myeloma line U-266 (Gabrea, 1999) (FIG. 2c).

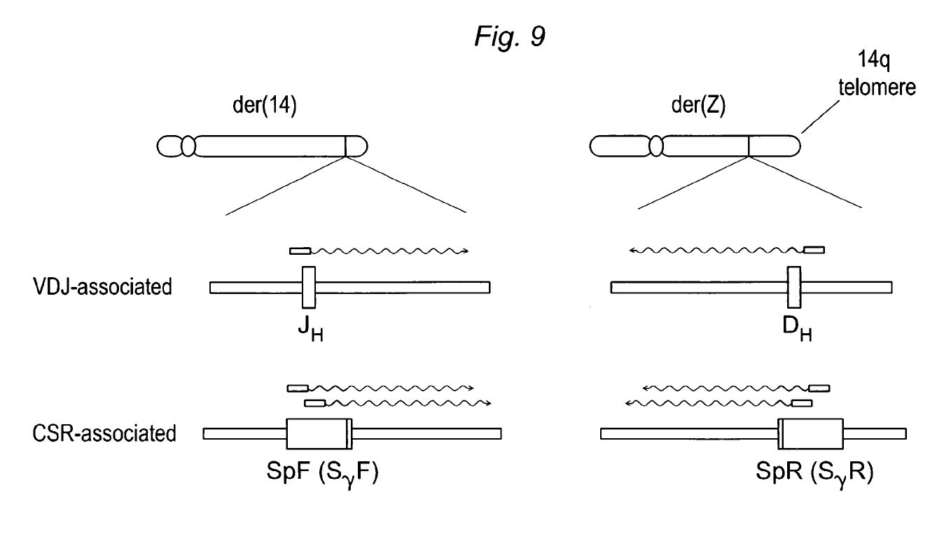

The large cell lymphoma cell line OCI-Ly8 (Tweeddale, Lim, et al., Blood 69(5):1307-1314 (1987)) is known to have a MYC rearrangement in addition to J_{H}-BCL2 and S_{γ3}-BCL6 fusions (Farrugia, Duan, et al., Blood 83(1):191-198 (1994); Chang, Blondal, et al., Leuk Lymphoma 19(1-2):165-71 (1995); Ye, Chaganti, et al., EMBO 14(24):6209-17 (1995)). Molecular cytogenetic studies have confirmed the presence of complex rearrangements of chromosomes 3, 8, 14 and 18 (Changanti, Rao, et al., Genes, Chromosomes and Cancer 23(4):328-336 (1998); Mehra, Messner, et al., Genes, Chromosomes and Cancer 33(3):225-234 (2002); Sanchez-Izquierdo, Buchonnet, et al, Blood 101(11):4539-4546 (2003)) but the details of the MYC rearrangement have not been completely established. Using tCGH with the J_{H} primer, we confirmed the J_{H}-BCL2 fusion. Interestingly, a large deletion in BCL2 intron similar to that identified in the RL7 cell line was incidentally identified, as well as a small breakpoint-associated deletion. We then designed a consensus linear amplification primer to recognize all S_{γ} repeats that we designated S_{γ}R, where R denotes a “reverse” orientation indicating that the 3′ end of the primer points towards the 14q centromere (FIG. 9)). tCGH performed using the S_{γ}R primer successfully identified the S_{γ3}-BCL6 fusion (FIG. 4b) and incidentally detected a previously unidentified S_{γ}-MYC fusion involving the 5′ end of the MYC gene (FIG. 4c). tCGH using a S_{γ}F linear amplification primer (F designates a “forward” orientation indicating that the 3′ end points towards the 14q telomere) successfully identified the reciprocal S_{γ}-BCL6 fusion (FIG. 4d). However, a reciprocal S_{γ}-MYC fusion could not be identified by tCGH performed with the S_{γ}F, SₚF, SₚR, J_{H} or D_{H} linear amplification primers.

### Example 3

### Identification of Unknown Translocation Breakpoints

To demonstrate that tCGH is capable of identifying novel breakpoints in primary tumors, we studied a series of lymphomas presumed to have J_{H}-CCND1 translocations. Mantle cell lymphoma (MCL, reviewed in (Jares, 2007) is a mature B cell lymphoma that is characterized by the t(11;14)(q13;q32), a translocation that results in a J_{H}-CCND1 gene fusion and over-expression of the CCND1 protein. While about 40-50% of MCL cases, including the MO2058 cell line (FIG. 3c), have breakpoints that cluster at the major translocation cluster (MTC), most MCL breakpoints are scattered throughout a large intergenic region between CCND1 and the MYEOV gene, located ˜400 kb centromeric to CCND1. To demonstrate that tCGH can detect as yet unidentified translocation breakpoints, we used it to identify and map J_{H}-CCND1 translocations in MCL cases that did not have MTC-associated breakpoints. FIG. 6 shows the results of tCGH using the J_{H} primer to study four such MCL cases and one t(11;14)-positive B cell prolymphocytic leukemia (B-PLL). In each case, a unique breakpoint was predicted at sufficient resolution to enable the immediate design of a PCR primer for amplification and sequencing of the unique J_{H}-CCND1 fusion. The breakpoints are scattered over a ˜140 kb segment of the CCND1 breakpoint region (Vaandrager, 1996), including one case (the B-PLL) having a breakpoint just outside the MTC.

### Example 4

### Methods and Materials

Genomic DNA preparation and hybridization: Test and reference DNA were each linearly amplified using one or more J_{H}, D_{H}, or S_{H} primers to target the IgH locus, MYC, BCL2, or BCL6 primers to target rearrangements at these loci, or one or more primers to target rearrangements involving the BCR, MYH11, MLL, PML, or AML/RUNX1 loci (see Tables 3 and 5). The amplified DNA was fragmented by sonication and differentially labeled with Cyanine-3-dUTP and Cyanine-5-dUTP (Agilent Genomic DNA Labeling Kit) as described below in [0125]. Labeled test and reference DNA samples were co-hybridized to Agilent Custom HD-CGH 8×15K Microarrays (Product Number G4427A) essentially as recommended by the manufacturer (Agilent Publications No. G4410-90010 and 64427-90010). Array data was analyzed using Agilent Feature Extractor software (version 9) and Agilent CGHAnalytics software (version 3.4 or 3.5).

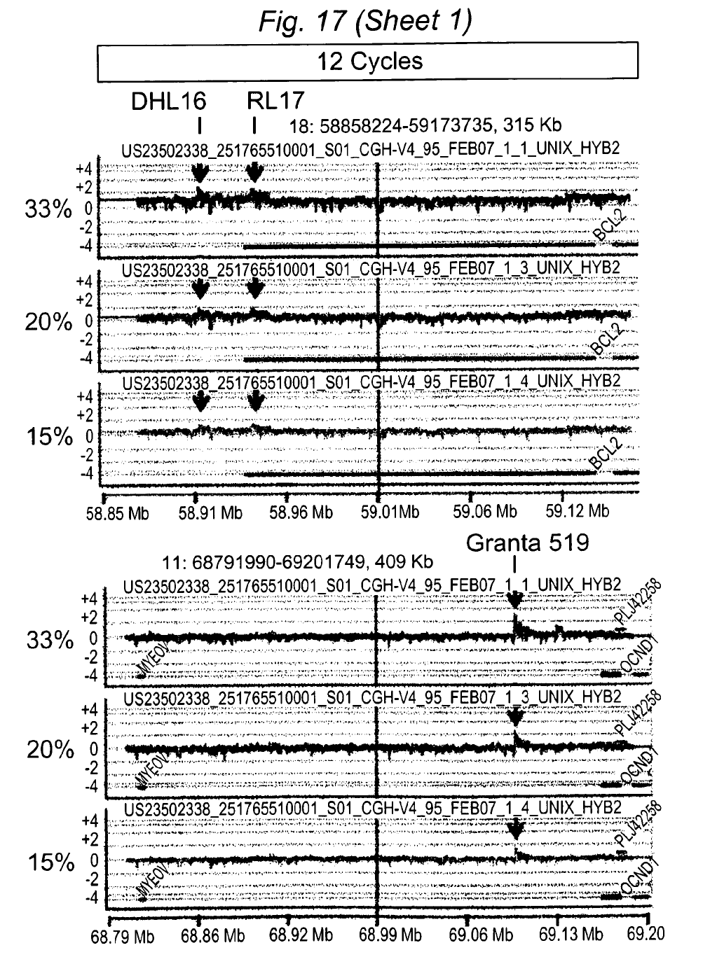

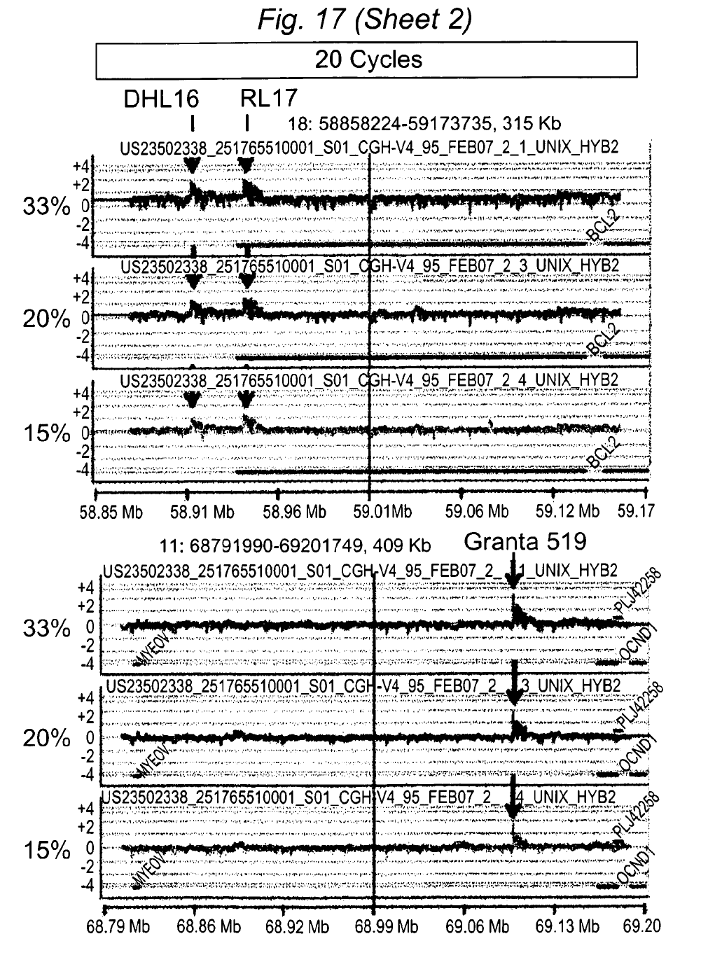

Linear amplification: Linear amplification reactions (50 μl) containing 0.5 μg to 2 μg genomic DNA, 200 mM dNTPs, and 150 nM linear amplification primer were amplified using the Clontech Advantage 2 PCR Enzyme System. Multiplex reactions contained 75 nM of each individual linear amplification primer. Typical reaction conditions were as follows: denaturation at 95° C. for 5 min followed by 12 cycles of denaturation at 95° C./15 sec, annealing at 60° C./15 sec, and extension 68° C./6 min, although extension times ranging from 2 min up to 18 min were also used successfully and up to 20 amplification cycles were performed in select experiments (e.g. see FIG. 17). After linear amplification, the resulting DNA mixture was fragmented by sonication using Fisher Model 550 Sonic Dismembrator fitted with a Misonix 431A cup horn in 400 μl of TE pH 8.0 for 3 minutes on ice and then concentrated (Microcon Y30) to a final volume of 32 μl. Fluorescent labeling and hybridization were performed using the Agilent Genomic DNA labeling kit PLUS (Cat #5188-5309) essentially as recommended by the manufacturer, except that neither restriction digestion nor whole genome amplification was performed. In general, labeling was performed by random primer mediated extension using a DNA polymerase in the presence of labeled dNTPs. This results in the labeling of both the linearly amplified products as well as the genomic DNA in the sample. However, the products of the linear amplification reaction can be solely labeled by the inclusion of labeled dNTPs in the amplification reaction. Control human genomic DNA was obtained from Promega (Cat #G1471 (male) and G1521 (female)).

Array Design: IgH or myeloid leukemia (AML) breakpoint-associated genomic regions were represented on Agilent DNA microarrays (G4427A) at tiling density by custom oligonucleotide probes that were selected using an algorithm designed to optimize parameters such as probe length, predicted melting temperature and probe spacing and density. Genomic regions selected for high-density representation by custom probes were first filtered using RepeatMasker (http://repeatmasker.org/cgi-bin/AnnotationRequest) to mask highly conserved repetitive sequence elements. To maximize genomic coverage, highly divergent repeats (>15% divergent) were not masked, since unique oligonucleotide probes could be identified in these regions. A uniform spatial distribution of probes across each repeat-masked genomic segment was generated using the program Tile (see below). The IgH translocation array contained a total of 11,852 probes representing five genomic loci commonly represented in IgH translocations: BCL2, BCL6, CCND1, MLT1, and MYC (see Table 1 below). In addition, 2410 control probes representing an additional 23 genomic loci at lower density were selected from an Agilent probe library (http://earray.chem.agilent.com/earray/). The AML array (see Table 4 below) contained a total of 14,262 probes representing the following genomic loci: BCR, ABL, ETO (RUNX1T1), AML1, RARA, PML, CBFB, MYH11, MLL, AF9, IKZF1 (Ikaros).

Tile (N. Hoffman and H. Greisman, unpublished) uses a simple algorithm to generate a uniform spatial distribution of oligonucleotides with melting temperature (Tm), GC content, and length in nucleotides as close as possible to specified parameters. The input is a list of oligonucleotide sequences, each associated with a start and end position in the genomic segment of interest and a melting temperature. For the arrays used in these experiments, candidate oligonucleotides included all possible N-mers spanning the region of interest, where N ranged from 25 to 60 (for the IgH array) or from 35 to 60 (AML array).

Parameters used in the oligonucleotide selection criteria are defined as follows:
Dₘₐₓ, Dₘᵢₙ, Dₒₚₜ—maximum, minimum and optimal distance from the starting position
Tmₘₐₓ, Tmₘᵢₙ, Tmₒₚₜ—maximum, minimum, and optimal Tm
Tmₘₐₓ, Tmₘᵢₙ, Tmₒₚₜ—maximum, minimum, and optimal Tm

The oligonucleotide selection algorithm proceeds iteratively as follows, starting at nucleotide position P₀ in the sequence region:

1. The set of oligonucleotides between nucleotide positions P₀+Dₘᵢₙ to P₀+Dₘₐₓ are considered as a group.
2. Oligonucleotides with a Tm outside the range [Tmₘₐₓ, Tmₘᵢₙ] are removed.
3. For each oligonucleotide i at position Pᵢ, the following values are calculated:

Dᵢ=|P₀+Dₒₚₜ−Pᵢ| a.

Thus smaller values correspond to a position closer to the distance optimum for the sequence region. Dᵢ may be rounded to the nearest D_{bin} nucleotides (typically a value of 5 is used).

dTmᵢ=|Tmᵢ−Tmₒₚₜ| b.

Thus smaller values of dTmᵢ correspond to a melting temperature closer to the Tm optimum. dTmᵢ may be rounded to the nearest dTm_{round} degrees (typically a value of 1 degree is used).

−Lᵢ=−1*(oligo length) c.

Thus smaller numbers correspond to longer oligonucleotides.

GCᵢ=100*(G+C)/length d.

Thus smaller numbers correspond to a lower G+C content. GCᵢ was rounded to the nearest percent.
4. For each oligonucleotide i, a list of tuples comprised of some or all of Dᵢ, dTmᵢ, −Lᵢ, and GCᵢ is created.
5. This list of values is sorted in ascending order. The relative position of each of Dᵢ, dTmᵢ, −Lᵢ, and GCᵢ in the tuple determines the relative weight of that parameter (i.e., if Dᵢ precedes GCᵢ, then oligonucleotide length is given more weight then GC content).
6. An oligonucleotide at position x corresponding to the first tuple in the sorted list is selected.
7. P₀ is reset to Pₓ, and the process is repeated.

The algorithm also addresses regions of discontinuity in the coverage of candidate oligonucleotides over the sequence of interest. Consider a sequence region spanning the range [P₀+Dₘᵢₙ, P₀+Dₘₐₓ], and a “gap” (that is, a sequence region containing no candidate oligonucleotides) spanning the range [P_{gapstart}, P_{gapstop}]. If P_{gapstart}<P₀+Dₘₐₓ, then in this iteration an oligonucleotide will be chosen from the range [P₀+Dₘᵢₙ, P_{gapstart}]. At the next iteration, oligonucleotide selection will start after the gap, and P₀ is set to (P_{gapstop}−Dₒₚₜ) to force the consideration of oligonucleotides closer to the point at which coverage of candidate oligonucleotides resumes.

The optimum probe length was 60 bases, the optimal distance between probes was between 50 and 100 bases, acceptable GC content was 20% to 80%, and optimal Tm was 74.5° C., calculated using the program Dan from the EMBOSS software suite (http://emboss.sourceforge.net).

TABLE 1

| | | | Region | | |
|---|---|---|---|---|---|
| Locus | Band | Region Interval | length | # probes | Spacing |
| CCND1 | 11q13.3 | chr11: 68808198- | 380,225 | 5,597 | 68 |
| | | 69188422 | | | |
| BCL2 | 18q21.33 | chr18: 58880000- | 277,594 | 4,213 | 66 |
| | | 59157593 | | | |
| MYC | 8q24.21 | chr8: 128782854- | 50,000 | 728 | 69 |
| | | 128832853 | | | |
| BCL6 | 3q27.3 | chr3: 188921902- | 53,280 | 847 | 63 |
| | | 188975181 | | | |
| MLT1 | 18q21.31 | chr18: 54468326- | 33,203 | 467 | 71 |
| | | 54501528 | | | |

### Confirmation of Novel Breakpoints:

Novel translocation and deletion breakpoints were all confirmed by PCR amplification and Sanger sequencing, including the J_{H}-CCND1 fusions in all 5 MCL cases, J_{H}-BCL2 fusions in one follicular lymphoma (FCL) case and in DHL16, and all four novel IgH fusions in OCI-Ly8 (J_{H}-BCL2, D_{H}-BCL2, S_{γ2}-MYC, and S_{γ3}-BCL6). Partner breakpoints are provided in Table 2 and sequences are provided below. In addition, the novel intronic BCL2 deletions in RL7 (chr18:58,954,729-59,122,208) and in OCI-Ly8 (chr18:58,998,604-59,133,954) were each amplified and sequenced using specific BCL2 primers that flank each deletion, see below.

TABLE 2

| | | | | Partner Breakpoint | |
|---|---|---|---|---|---|
| Cell line/case | IgH primer | IgH Breakpoint | Partner | (hg17) | Diagnosis |
| MO2058 | J_{H} | J_{H}4 | CCND1 | chr11: 69,055,996 | MCL |
| Granta 519 | J_{H} | J_{H}4 | CCND1 | chr11: 69,100,509 | MCL |
| U-266 | SpF (Sμ) | Sα1 | CCND1 | chr11: 69,153,045 | Myeloma |
| U-266 | SγR | Sγ2 | CCND1 | chr11: 69,153,019 | Myeloma |
| RL7 | J_{H} | | BCL2 | chr18: 58,944,489 | DLBCL |
| DHL16 | J_{H} | J_{H}6 | BCL2 | chr18: 58,914,890 | DLBCL |
| OCI-Ly8 | J_{H} | J_{H}6 | BCL2 | chr18: 58,944,475 | DLBCL |
| OCI-Ly8 | D_{H} | D_{H}3-10 | BCL2 | chr18: 58,938,252 | DLBCL |
| OCI-Ly8 | SγR | Sγ3 | BCL6 | chr3: 188,945,670 | DLBCL |
| OCI-Ly8 | SγF | Sγ3 | BCL6 | chr3: 188,945,699 | DLBCL |
| OCI-Ly8 | SγR | Near Sγ2 | MYC | chr8: 128,818,596 | DLBCL |
| MC116 | J_{H} | J_{H}4-J_{H}5 | MYC | chr8: 128,817,581 | MYC |
| Raji | SpF (Sμ) | | MYC | chr8: 128,816,104 | MYC |
| MCL1 | J_{H} | J_{H}4 | CCND1 | chr11: 69,131,130 | MCL |
| MCL2 | J_{H} | J_{H}6 | CCND1 | chr11: 69,059,199 | MCL |
| MCL3 | J_{H} | J_{H}5 | CCND1 | chr11: 68,989,831 | MCL |
| MCL4 | J_{H} | J_{H}5 | CCND1 | chr11: 69,082,854 | MCL |
| MCL5 | J_{H} | J_{H}4 | CCND1 | chr11: 69,056,460 | MCL |
| FCL | J_{H} | J_{H}6 | BCL2 | chr18: 58,944,421 | FCL |

PCR/sequencing primers and breakpoint sequence for MCL1 J_{H}-CCND1 fusion:

(SEQ ID NO: 27)
5′-GACCCAGCACCCTTATTTCC-3′ (IgH)
(SEQ ID NO: 28)
5′-GATCACAGTCTTTGCTGCCTGT-3′ (CCND1)
(SEQ ID NO: 29)
CAGTTTTAGAGTTGTTTGTGGCAGGAAAGTTACTTTTGGCCAGAATTGGA
AGTTGGAAGGTGTGCAGCTATTGCTATAGCAAATGTGTTCTCCATCCTGA
TCAGTAAAGAGGATAAAAAGCAATTTATCATTAGATAGGAAGGATATTCA
CAATCTCACTCCAGATCTATGTTATAATAACTCCTGTTCTCCAAAGAATA
TAGGTTGACTACTGGGGCCAGGGAACCCTGGTCACCGTCTCCTCAGGTAA
G

PCR/sequencing primers and breakpoint sequence for MCL2 J_{H}-CCND1 fusion:

(SEQ ID NO: 30)
5′-CCAGGCTCAGTTACTCCATCAG-3′ (IgH)
(SEQ ID NO: 31)
5′-CACTCTGGAACATTCTTGCATTG-3′ (CCND1)
(SEQ ID NO: 32)
TGTAATCCCCAGCACTTTGGGAGGCTGATACGGGAGAATCACTTAGCCCC
AGAGAAGTTCCAAGAACAGCCTGGGCATCATACATAGCGAGACTCGTTCT
CTAAAAAATACAAAAAAATTAGCTGGGTGTGGTGGCACGTGCCTGTAGTC
ACAGCTACTTGGGAGGCTGTGATGGGAGGATCACTGGAGCCCAGGAACTC
CAGGCTGTAGTGAACTATGATCATGCCACTGCTCCAGCCTGGGTGACAGT
GTGAGACCCTGTCTCTGATAATAATCATAATATTTTATTAGTAGAGTCGT
TTTTTCTTTTTCATTTCTTTTTAATTTAATGTTTTGTACGGACAAGTTTT
CGCTATTTTGCCCAGGCTAGTCTTGAACTCCTGGCCTCAACCGATCCTCC
TGCCTCAGCCTCCCAAAGCACTGGGATTACAGGCAAGAACCACCGCACCC
GGCCCAAACATTTTCATTTTTTATATTTCAAGTACTTTGATTAATTATTG
TGCAAGTTTCTTGTGCAAAGCTTAGAAGAAGAGGTCTTACAGAATTTTTT
GCGGTTTTTAAGCAATTACACCATATAAAACTACTACTACTACGGTATGG
ACGTCTGGGGCCAAGGGACCACGGTCACCGTCTC

PCR/sequencing primers and breakpoint sequence for MCL3 J_{H}-CCND1 fusion:

(SEQ ID NO: 33)
5′-AGAGGCTCCCAGATCCTCAAG-3′ (IgH)
(SEQ ID NO: 34)
5′-AACACAGTGCCATGAAACCA-3′ (CCND1)
(SEQ ID NO: 35)
AACACTAGATCTGGAAAATAGGGTTTCATGGCCCAGAGTTTGGGGAACCG
GGCACGAGAGCTGAGTCAGCATCTTTGGCTGTGAAAAATCTCTGCTTAAT
GTTGGCTGCCACGGGCTCCACAGCCTCTTTGCCATGGCATATCTTCTGGC
GTGCCACTGACTCACACCATCATGAAATGAGCGCCATGCAGAACACAATC
TTGGGGAAAAGCTTATCTAAGGAATAAAATTACAGGTCCAGATCACTTAT
ATAACTGGCGCTTAGAAGAAGAGCCCACCCACATTATTTTTTGAGGACCC
CCATGGCTGATGGTGAACCCCTGCTCTGAGGTGGGTGGCTTCCCTTCAGC
CCCACGGCTTGTTGGGGGGCACCCTCCACCCAGCTGTCGCCTTGGGGTAA
GGCTCCCCACGGAGCCACCCGAAATGAATCTGGTTCGACCCCTGGGGCCA
GGGAACCCTGGTCACCGTCTCCTCAGGTGAGTCCTCACCACCCCCTCTCT
GAGTCCACTTAGGGAGACTCAGCTTGCCAGGGTCTCAGGGTCAGAGTCTT
GGAGGCATTTTGGAGGTCAGGAAAGAAA

PCR/sequencing primers and breakpoint sequence for MCL4 J_{H}-CCND1 fusion:

(SEQ ID NO: 36)
5′-CCAGGCTCAGTTACTCCATCAG-3′ (IgH)
(SEQ ID NO: 37)
5′-CTGTGACCACTTCCTGACCA-3′ (CCND1)
(SEQ ID NO: 38)
CTTTAGGTACACGGATGGAGGTCTGCTGGCCGAGGCTGGCATTTGCACAC
TTCATTGCAGCACTGGAGAACTGAGTCTTCTTTTACTCAATTTTTCATAG
AAATAGGCACATTCCCCATCCCTTCCCCTCGCCCCCACAACCCCTGACGC
TCAGCATCCAGGGCTGATCTGAGAGGGACCCGGGAGGCAGAGAAACCCCA
GAGCCGTCATTTCCCAGATGTGGCATTATGTGTGAGCCTAGGTTTGTGTT
CTTTTAACGGCACCACATAAACCCCAGTCCTCCAAACTGGTTCGACCCCT
GGGGCCAGGGAACCCTGGTCACCGTCTCCTCAGGTGAGTCCTCACCACCC
CCTCTCTGAGTCCACTTAGGGAGACTCAGCTTGCCAGGGTCTCAGGGTCA
GAGTCTTGGAGGCATTTTGGAGGTCAGGAAAGAAAGCCGGGGAGAGGGAC
CCTTCGAATGGGAACCCAGCCTGTCCTCCCCAAGTCCGGCCACAGATGTC
GGCAGCTGGGGGGCTCCTTCGGCTGGTCTGGGGTGACCTCTCTCCGCTTC
ACCTGGAGCATTCTCAGGGGCTGTCGTGATGATTGCGTGGTGGGACTCTG
TCCCGCTCCAAGGCACCCGCTCTCTG

PCR/sequencing primers and breakpoint sequence for MCL5 J_{H}-CCND1 fusion:

(SEQ ID NO: 39)
5′-GACCCAGCACCCTTATTTCC-3′ (IgH)
(SEQ ID NO: 40)
5′-ACCACACCTGGCCTTCTATTGTA-3′ (CCND1)
(SEQ ID NO: 41)
TGTCTAATGCCCTGTATCCCCATTTTAACATCATACAAAGAGTTTCACTG
CCCTAAAAATCTGTCTCCACCTGTTCACCCCTCTCTCCAAATTCCTGGCA
ACCACTGATTGTTTTACTTTCTCTGTAGTTTTGTCCTTTCTAGAATGTCA
AAGAGTTGGACTCATACGGTAACGGAGAGACCAGCATACATGACTACTGG
GGCCAGGGAACCCTGGTCACCGTCTCCTCAGGTGAGTCCTCACAACCTCT
CTCCTGCTTTAACTCTGAAGGGTTTTGCTGCATTTTTGGGGGGAAATAAG
GGGCTGGGTC

PCR/sequencing primers and breakpoint sequence for FCL J_{H}-BCL2 fusion:

(SEQ ID NO: 42)
5′-CTTACCTGAGGAGACGGTGACC-3′ (IgH)
(SEQ ID NO: 43)
5′-CGGGAATTCTTTGACCTTTAGAGAGTTGCTT-3′ (BCL2)
(SEQ ID NO: 44)
TTTTCCAAGGCATCGGAAATCCACAGAGGCTCCCAGATCCTCAAGGCACC
CCAGTGCCCGTCCCCTCCTGGCCAGTCCGCCCAGGTCCCCTCGGAACATG
CCCCGAGGACCAACCTGCAATGCTCAGGAAACCCCACAGGCAGTAGCAGA
AAACAAAGGCCCTAGAGTGGCCATTCTTACCTGAGGAGACGGTGACCGTG
GTCCCTTGGCCCCAGACGTCCATCACCGGGCCCCGCCGGGGAGGTCTGGC
TTCATACCACAGGTTTCCTGCTTTCTTGGTGGAGCGTAAGCACCACTGCA
TTTCAGGAAGACCCTGAAGGACAGCCATGAGAAAGCCCCCGCGGAAGGAG
GGCAGGAGGGCTCTGGGTGGGTCTGTGTTGAAACAGGCCACGTAAAGCAA
CTCTCTAAAGGTCAAA

PCR/sequencing primers and breakpoint sequence for DHL16 J_{H}-BCL2 fusion:

(SEQ ID NO: 45)
5′-CCAGGCTCAGTTACTCCATCAG-3′ (IgH)
(SEQ ID NO: 46)
5′-CGGGAATTCTCAGTCTCTGGGGAGGAGTGG-3′ (BCL2)
(SEQ ID NO: 47)
ACAGGCAGTAGCAGAAAACAAAGGCCCTAGAGTGGCCATTCTTACCTGAG
GAGACGGTGACCGTGGTCCCTTGGCCCCAGACGTCCATATTAATATTTGG
CGAGACAGAGAATACCACAAAGAAGTGGATAGATGGCAGATGACACATGC
GAGACCCAAAGTGCTAATTTCCTGAATATACAAAGAGCTCTTACAAGTTA
ATCAAAAGACAAATAACTCAATGAAGTAATGGGCTCTTGCCTATGTTATG
AATATTTTCATCTGGTATGACATTTACTCTTTGATTTCATTATTTTTGTG
ATTTTCATAGGCTTGTATTTTATGTAGTCAAATCTCTATGTCTTTCTATC
ACTTTTGTGTTTAGAAGGACGTTCCTCCTTCTGAAGTACACATATTTGAT
TTTGGATTTGAGATGGCATTCATTTCTGATCCATCTATAAAGTTTTTGGT
GTGTGGTTAAAGGTAAAAATAGACTCACATTTTCCTAAATAATTAGCCAG
TTGTTCCAGCTCCATTTATTGAGTGGTCCTTCCTTTCCCAACTGATGCGC
GGTGTAACCTTTATCATATATTAAATACTCATGTGTGCTAAAAATAAAAA
GTCATTTCAGTTGAGTGCTG

PCR/sequencing primers and breakpoint sequence for OCI-Ly8 J_{H}-BCL2 fusion:

(SEQ ID NO: 48)
5′-CCAGGCTCAGTTACTCCATCAG-3′(IgH)
(SEQ ID NO: 49)
5′-CGGGAATTCTTTGACCTTTAGAGAGTTGCTT-3′ (BCL2)
(SEQ ID NO: 50)
TTTTCCAAGGCATCGGAAATCCACAGAGGCTCCCAGATCCTCAAGGCACC
CCAGTGCCCGTCCCCTCCTGGCCAGTCCGCCCAGGTCCCCTCGGAACATG
CCCCGAGGACCAACCTGCAATGCTCAGGAAACCCCACAGGCAGTAGCAGA
AAACAAAGGCCCTAGAGTGGCCATTCTTACCTGAGGAGACGGTGACCGTG
GTCCCTTGGCCCCAGACGTCCATACCGTATTTTCATCCCATTCGCACACA
GGGGGTAACGGGGCGCCGGGTAAGCACCACTGCATTTCAGGAAGACCCTG
AAGGACAGCCATGAGAAAGCCCCCGCGGAAGGAGGGCAGGAGGGCTCTGG
GTGGGTCTGTGTTGAAACAGGCCACGTAAAGCAACTCTCTAAAGGTCAAA
GAATTCCCGA

PCR/sequencing primers and breakpoint sequence for OCI-Ly8 D_{H}-BCL2 fusion:

(SEQ ID NO: 51)
5′-CTGGAGCACTTCAACAGCAG-3′ (BCL2)
(SEQ ID NO: 52)
5′-GTGGCCCTGGGAATATAAAA-3′ (IgH)
(SEQ ID NO: 53)
ATTTGTGGGCACTTATGAACCCGAAAGGACATGGCCATGGGGTGGGTAGG
GACATAGGGACAGATGCCAGCCTGAGGTGGAGCCTCAGGACACAGGTGGG
CACGGACACTATCCACATAAGCGAGGGATAGACCCGAGTGTCCCCACAGC
AGACCTGAGAGCGCTGGGCCCACAGCCTCCCCTCAGAGCCCTGCTGCCTC
CTCCGGTCAGCCCTGGACATCCCAGGTTTCCCCAGGCCTGGCGGTAGGTT
TAGAATGAGGTCTGTGTCACTGTGGTATTACGATATTTTGACTGGTTATT
ATAACCACAGTGTCACAGAGTCCATCAAAAACCCATGCCTGGAAGCTTCC
CGCCACAGCCCTCCCCATGGGGCCCTGCTGCCTCCTCAGGTCAGCCCCGG
ACATCCCGGGTTTCCCCAGGCTGGGCGGTAGGTTTGGGGTGAGGTCTGTG
TCACTGTGGTATTACTATGGTTCGGGGAGTTGTACGGCCCTGATACTTAG
CTTCCATTGTATACATTTTTAAAGTGATATAAAACAAATCTGGTTGTGAT
TCCTATCAACATAGGCATGAGCCACTGCGCTCAGCCCTCTTTGTTTTTGT
GCTGGTTCCTTTGTGAAAGTTCTGCTGTTGA

PCR/sequencing primers and breakpoint sequence for OCI-Ly8 Sγ2-MYC fusion:

(SEQ ID NO: 54)
5′-GGTCGGACATTCCTGCTTTA-3′ (MYC)
(SEQ ID NO: 55)
5′-CCCAGAAGGAGCAAGATGG-3′ (IgH)
(SEQ ID NO: 56)
GGGTTTGGGGGGCTGGGGGTTGCTTTGCGGTGGGCAGAAAGCCCCTTGCA
TCCTGAGCTCCTTGGAGTAGGGACCGCATATCGCCTGTGTGAGCCAGATT
GCTCCGCAGCCGCTGACTTGTCCCCGTCTCCGGGAGGGCATTTAAATTTC
GGCTCACCGCATTTCTGACAGCCGGAGACGGACACTGCGGCGCGTCCCGC
CCGCCTGTCCCCGCGGCAAGGTCCCTGTTGGGCTCAACCCAGGCCCCCCA
GCATATGTAGGAGCCTTGTATGGCCCTCCCCACCCTGCGTGGTGCCAGGA
CCCCCAGGCCACAGGGAGGCCCCATTTCTCTCTGCCGCTGGCCCAGTGGC
CCTGGAGTCCCACTCCACGTGGGGTGTGCCCCTGACTTCTGAGGAACCTA
AGTGCCCTGCCCTCAGCCAGGCCATCCCCTCTGCTCAGAGGGCCCCGCTC
ACCACCCCTTCCCCTCACCTGCAGCACAGACTCTGGCTGATTCTGCCCAG
GCCCTGAATGGGCCCCTCTGGCAGCCGTCTGTTGCTACACTGCCC

PCR/sequencing primers and breakpoint sequence for OCI-Ly8 Sγ2-BCL6 (SγF) fusion:

(SEQ ID NO: 57)
5′-CCTGCCTCCCAGTGTCCTGCATTACTTCTG-3′ (IgH)
(SEQ ID NO: 58)
5′-GCAGTGGTAAAGTCCGAAGC-3′ (BCL6)
(SEQ ID NO: 59)
ACTGGGGTTCTTAAAGTGGTGATGCAAGAAGTTTCTAGGAAAGGCCGGAC
ACCAGGTGATTATTGCTGTTGCTGCCGCCGCTGCTGCTGCTACTGCCGCC
GCCGCCGCTGTTGCCGCTGGTGCCGCTGCCGCCGCCGCTGCTCATGATCA
TTATTTTACCTTTTAATTCTTTTTTTTTCCGCTCTTGCCGAGTGCTTTGG
CTCCAAGTTTTCTATGTGTATCTATTGATATAAATGTATATATTTATTTA
TTCTAGGTGGAGCTCARAGAGCCGGGGAARATCATCGGTAGGTGAGCAGG
GGCTGGTGGAAAGCAGGAGGAGCAAGGGGCAGCTCATGGAGCTCAGAGGA
CCAGGGAAGAGCAGCCACAGGTGAACAGGGGCAGGTGGGCGGCAGAATGA
GCAGGGGCAACTCCTGGAGCTCAGGGGACAAGGGCAGAGCAGCCATAGGC
AAACAGGAGCAGGGTCAGGGGACAGGAGGAGCAGGGGGCAGTTCTTGGAG
TTTAGGGGACCAGGGCAGAGCCGCTGCAGGTGAGCAGGGGCAGGTGGGGG
GCAGGAGGAGCAGGGGGCATCTCCTGGAGCTCAGAGCACCAGGGCAGAGC
AGCCACAGGTGAGCAGGGACGGTGGGAGGCAGCACGCAGCTCCTAGACTT
TGGCAGGAGCTGGGTAGTTGCCGGCACCAGACAGCTGAGGGCTGGTGAAA
GTGCAGTGCAGCCTCCTGGTG

PCR/sequencing primers and breakpoint sequence for OCI-Ly8 intronic BCL2 deletion:

(SEQ ID NO: 60)
5′-TCAGACACCAGCTCCCTAGC-3′
(SEQ ID NO: 61)
5′-GAAGCACAGATGGTTGATGG-3′
(SEQ ID NO: 62)
AGGAAGTCTTGCTCTTCAGCAAAAACTGCAGCAGGAATCCCTAAATGCCT
AAAATGACTTTTAGATTAATAATGCACATTTGAAATGATAGGCAAATCAT
TCATCATTCTCAAACACTACCACTTTTATTCCAAAGTGTCCTATGAGTGC
ATTTCCTGGTATATATATATATTTTTTCTCTAAATTCCTTTGTTTTCCTA
TATAGTCACCATCTATGCATTGTTCTGAAAGCTATTACATATTTTACCAT
CAACC

PCR/sequencing primers and breakpoint sequence for RL7 intronic BCL2 deletion:

(SEQ ID NO: 63)
5′-CTCATGCCCCATATTCATTCAA-3′
(SEQ ID NO: 64)
5′-AGGGCATGTTACTGCAAGTTCA-3′
(SEQ ID NO: 65)
ATTTGTTTACTGTAAAGAGCCTACTCTGGGCCAATCATTGTTCTAGGGGC
TGGAGCTGTAACAGAAAACATGACAGGTTAAGAACTCTCCCATCTCTTGT
GAATCCCACATTGTGAGGAAGTCAGAAAAGTAAACAACTTAAAAATTAAC
AATATAACATAAGGGCCACAAAGTAATGAAAACAGGGCAATGCAGTGCAA
GGGTGCTGATGGGACAATATTAACTTCTTCCTTGGTCTTTGAGATTTTTC
AAGCAGTACTACAAGTTTACACAGAGGAGATTTAATGGGTTTTTCTTCAT
TAATAGTTGAAAACTATTTATAAGACAAATAATAATTTGTCTTAGGGTCT
GTTTCTAAGGGAGTCTAACCTATGGCCATGGGATTTACTGTTGAATGAAT
ATGGGGCATGAG

### Example 5

### Identification of Immunoglobulin Heavy Chain (IgH) Translocations

To render balanced rearrangements detectable on oligonucleotide arrays, breakpoint-associated genomic DNA sequences are enzymatically amplified prior to DNA labeling and array hybridization. This linear amplification step employs a single oligo primer to asymmetrically prime synthesis of hybrid DNA fragments that start in one genomic locus and extend across the translocation breakpoint into the partner locus (FIG. 7). Hybridization of the amplified and labeled genomic DNA to a tiling-density oligo array that is designed to represent the partner locus allows the translocation partner to be identified and the genomic breakpoint to be mapped to high resolution (FIG. 7). Because a second primer targeting the partner locus is not required for amplification, tCGH can detect translocation breakpoints scattered over large genomic regions and in multiple partner loci using a single array. Since amplified normal genomic DNA is used as the reference sample for array hybridization, tCGH can detect genomic imbalances and balanced translocations on the same array.

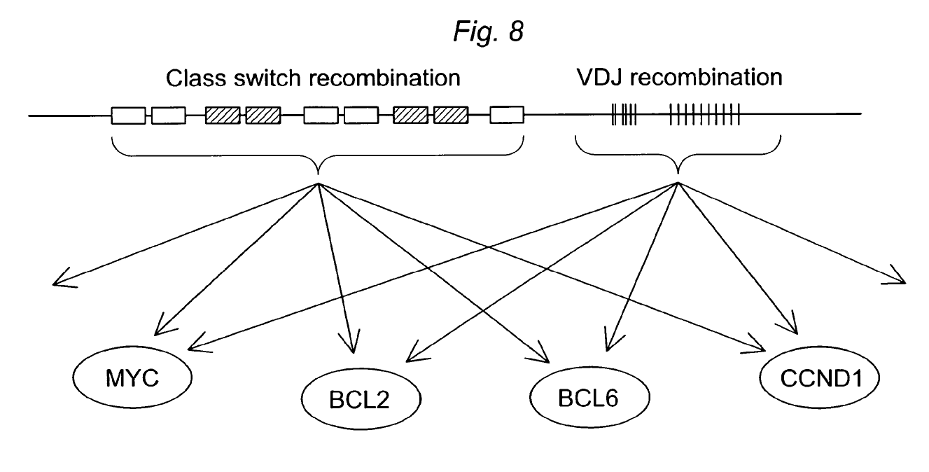

In the present example immunoglobulin heavy chain (IgH) translocations were studied, which can involve a variety of partner loci in various B cell lymphomas and in plasma cell myeloma, as a model system to develop and validate tCGH. Identification of the specific IgH partner locus in a particular lymphoma or myeloma is essential for accurate diagnosis and classification and for predicting clinical outcome and prognosis. Because IgH translocations are thought to arise as byproducts of aberrant VDJ or class switch recombination (CSR) and typically fuse the entire coding region of an oncogene to conserved IgH regulatory regions, breakpoints within the IgH locus tend to be located in conserved joining (J_{H}), diversity (D_{H}) or switch (S_{H}) segments (FIG. 8) while breakpoints in various IgH partner loci can be scattered across hundreds of kilobases of genomic sequence. These features of IgH translocations were exploited by designing a small set of linear amplification primers capable of detecting all of the conserved IgH breakpoint regions and designing high-resolution custom oligonucleotide arrays that represent multiple IgH partner loci at tiling density. The pilot array described here represents a total of about 1 Mb of genomic sequence including the CCND1 and BCL2 breakpoint regions and portions of the MYC and BCL6 breakpoint regions. The IgH locus is characterized by recent genetic duplications (Ravetch, Siebenlist et al., Cell 27(3, Part 2): 583-591; Matsuda, Ishii et al., J. Exp. Med. 188(11) 2151-2162 (1998)) and therefore was not represented on this pilot array.

A panel of eight lymphoma and myeloma cell lines with well-characterized IgH translocations involving BCL2, BCL6, MYC and CCND1 loci was used to test and validate tCGH. All nine known IgH translocations were identified and mapped to high resolution, as was a previously unknown IgH-MYC rearrangement and multiple copy number abnormalities at the IgH partner loci. In VDJ-associated translocations, IgH breakpoints on the der(14) chromosome typically map to one of six J_{H} segments while the breakpoints on the reciprocal chromosome map to one of 27 D_{H} segments. (van Dongen, Langerak et al., Leukemia 17(12): 2257-317 (2003)) J_{H} breakpoints and their reciprocal D_{H} counterparts were identified independently by linear amplification using either a single consensus J_{H} primer or an equimolar mix of 7 consensus D_{H} primers (FIG. 9). For example, tCGH analysis of a typical balanced IgH-BCL2 translocation in the lymphoma cell line DHL16 (Hecht, Epstein et al., Cancer Genetics and Cytogenetics 14(3-4):205 (1985)) shows reciprocal J_{H}-BCL2 and BCL2-D_{H} fusions (FIGS. 10a and 10b, respectively) that map to the BCL2 minor translocation cluster (van Dongen, Langerak et al., Leukemia 17(12): 2257-317 (2003)). The J_{H} and D_{H} fusions appear on fluorescence log-ratio plots as asymmetric pseudo-amplicons having a single distinct boundary that precisely marks the translocation breakpoint to high resolution. The amplitude of this amplicon gradually decreases with increasing genomic distance from the breakpoint, returning to the baseline over a span of several kilobases. The orientation of each amplicon depends on the direction of amplification: towards the telomere for the J_{H} primer (FIG. 10b) and towards the centromere for the D_{H} primers (FIG. 10b). In contrast, the amplitude, width, and shape of individual amplicons appear to vary with extrinsic parameters including the linear amplification primer, amplification conditions, and possibly local genomic sequence

The J_{H} and D_{H} primers also were used to map an IgH-MYC fusion in the Burkitt lymphoma cell line MC116 (FIG. 10f) and IgH-CCND1 fusions in the mantle cell lymphoma lines MO2058 and Granta 519 (FIG. 13a, 13d). The analytic sensitivity of tCGH was determined by mixing equal amounts of DHL16, RL7, and Granta 519 genomic DNA (designated “33% dilution”); 20% and 15% dilution samples were produced by mixing with normal genomic DNA. These dilutions were amplified for 12 or 20 cycles using the J_{H} primer and co-hybridized to similarly amplified normal genomic DNA. As shown in FIG. 17, all three breakpoints were detectable in each sample, although the signal for the 15% dilution was weaker when amplified for 12 cycles than 20 cycles.

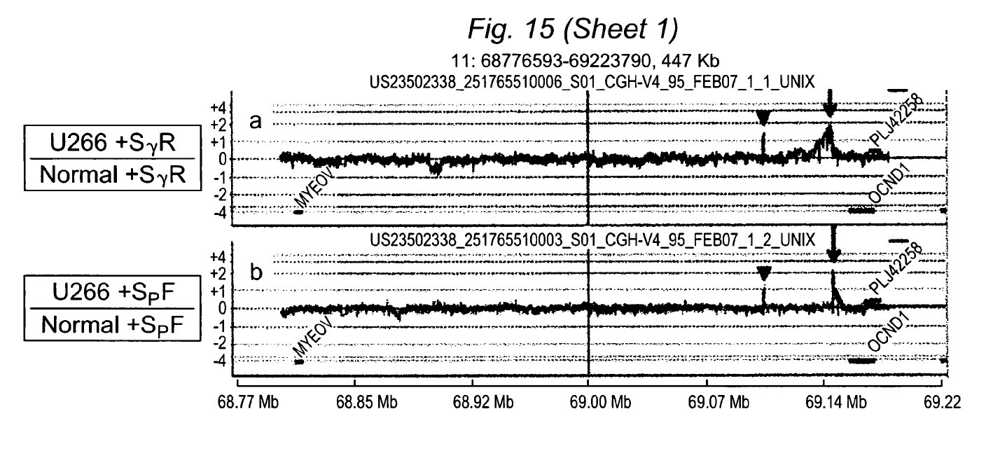

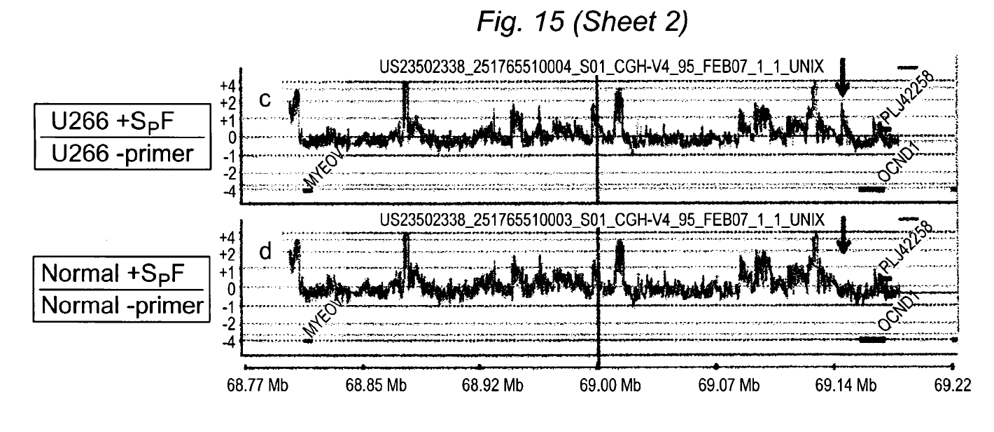

To identify S_{H}-associated translocations, reciprocal SₚF and SₚR amplification primers were designed to target the S_{μ}, S_{α}, and S_{ε} repeats, which all contain the pentameric sequence (GRGCT)ₙ, (Mills, Brooker et al., Nucleic Acid Res. 18(24):7305-16 (1990)) as well as reciprocal S_{γ}F and S_{γ}R primers to target the four closely related S_{γ} repeats (Mills, Mitchell et al., J. Immunol. 155(6):3021-3036 (1995)) (Table 3). S_{γ}F- or S_{γ}R-primed linear amplification of the OCI-Ly8 lymphoma cell line (Tweeddale, Lim et al., Blood 69(5):1307-1314 (1987) identified both reciprocal S_{γ3}-BCL6 fusions (FIGS. 10c and 10d), only one of which had been previously identified and cloned (Ye, Chaganti et al., EMBO J. 14(24) 6207-17 (1995)). Similarly, SₚF- and S_{γ}R-primed linear amplification of the myeloma cell line U266 (FIG. 15) identified a cryptic insertion into the CCND1 locus of a ˜100 kb IgH constant region segment that extends from S_{α1} to S_{γ4} and encompasses the 3′ α1 enhancer (Gabrea, Bergsagel et al., Molecular Cell 2(1):119 (1999)). Like the J_{R}-primed amplicons, those amplified using SₚF or S_{γ}F are oriented towards the telomere and correspond to der(14)-encoded fusions, whereas SₚR, S_{γ}R or D_{H}-primed linear amplification yields centromerically oriented amplicons corresponding to fusions on the reciprocal derivative chromosome (FIG. 9).

TABLE 3

| | | SEQ | |
|---|---|---|---|
| Primer | | ID | |
| name | Sequence | NO: | Reference |
| JH | CTT ACC TGA GGA GAC GGT | 1 | van Dongen, |
| | GAC C | | Langerak |
| | | | et al. 2003 |
| SpF | GCY CAG CYC AGC YCA | 2 | This study |
| SpR | GRG CTG RGC TGR GCT | 3 | This study |
| SγF | GGC TGC TCT GCC CTG GTC | 4 | This study |
| | CCC TGA GCT CCA | | |
| SγR | TGG AGC TCA GGG GAC CAG | 5 | This study |
| | GGC AGA GCA GCC | | |
| DH1 | GGC GGA ATG TGT GCA GGC | 6 | van Dongen, |
| | | | Langerak |
| | | | et al. 2003 |
| DH2 | GCA CTG GGC TCA GAG TCC | 7 | van Dongen, |
| | TCT | | Langerak |
| | | | et al. 2003 |
| DH3 | GTG GCC CTG GGA ATA TAA | 8 | van Dongen, |
| | AA | | Langerak |
| | | | et al. 2003 |
| DH4 | AGA TCC CCA GGA CGC AGC | 9 | van Dongen, |
| | A | | Langerak |
| | | | et al. 2003 |
| DH5 | CAG GGG GAC ACT GTG CAT | 10 | van Dongen, |
| | GT | | Langerak |
| | | | et al. 2003 |
| DH6 | TGA CCC CAG CAA GGG AAG | 11 | van Dongen, |
| | G | | Langerak |
| | | | et al. 2003 |
| DH7 | CAC AGG CCC CCT ACC AGC | 12 | van Dongen, |
| | | | Langerak |
| | | | et al. 2003 |
| Bcl6R | AGA ATT CCA GAG GCC GAG | 13 | Akasaka, |
| | CTT TGC TAC AGC GAA GG | | Akasaka et |
| | | | al. 2000 |
| Bcl2F | CTC ATG CCC CAT ATT CAT | 14 | This study |
| | TCA A | | |
| MycF | GGT CGG ACA TTC CTG CTT | 15 | This study |
| | TA | | |

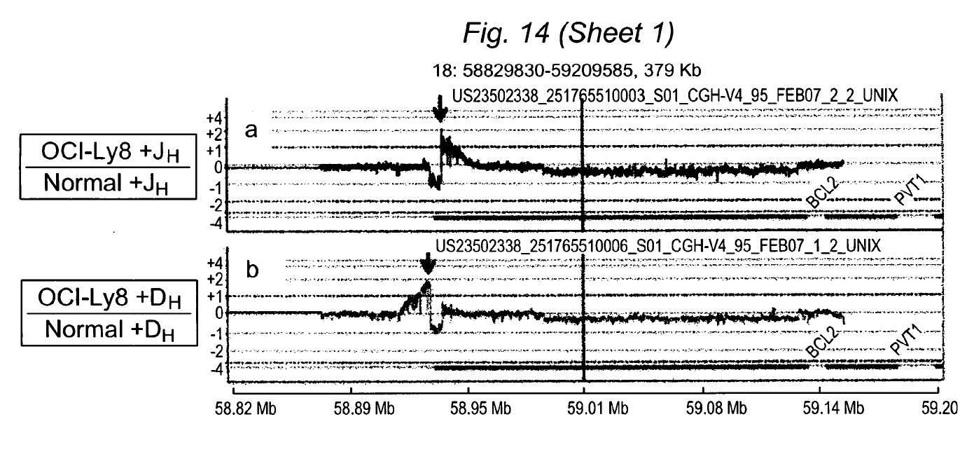

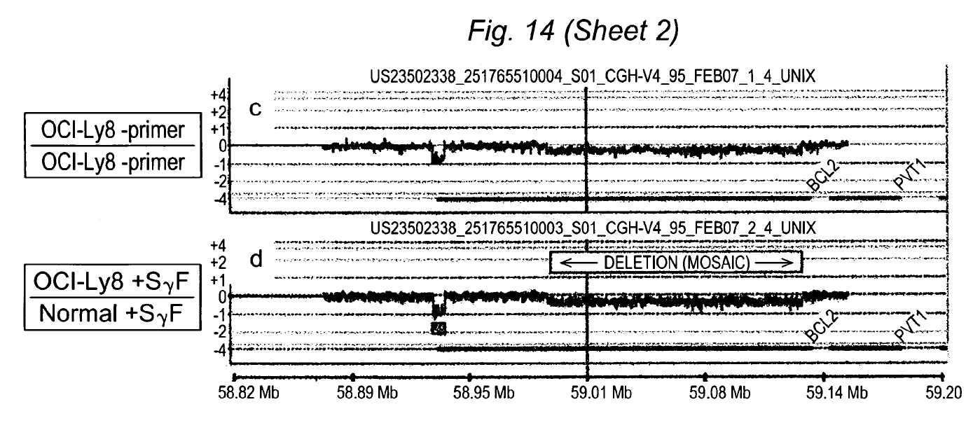

Surprisingly, in addition to identifying the known S_{γ3}-BCL6 translocation in OCI-Ly8, S_{γ}R-primed amplification also unexpectedly revealed a previously unidentified S_{γ2}-MYC fusion (FIG. 11e). Previous cytogenetic and molecular studies of OCI-Ly8 have shown complex rearrangements involving the BCL6, MYC, IgH and BCL2 loci on chromosomes 3, 8, 14 and 18, (Farrugia, Duan et al., Blood 83(1):191-198 (1994); Chang, Blondal et al., Leuk Lymphoma 19(1-2):165-71 (1995); Ye, Chaganti et al., EMBO J. 14(24) 6207-17 (1995); Changanti, Rao et al., Genes Chromosomes and Cancer 23(4):328-336 (1998); and Sanchez-Izquierdo, Siebert et al., Leukemia 15(9):1475-84 (2001)) although only one of these six potential IgH fusion products has been cloned and sequenced (Ye, Chaganti et al., EMBO J 14(24) 6207-17 (1995)). tCGH identified a total of five IgH fusions in OCI-Ly8, including both reciprocal fusion products of the balanced S_{γ3}-BCL6 translocation (FIG. 10) and the J_{H}/D_{H}-BCL2 translocation (see below and FIG. 10) as well as an apparently balanced S_{γ2}-MYC rearrangement (FIG. 11e). Interestingly, several unusual features of the translocation breakpoint sequences in OCI-Ly8, including an unusually large, about 6 kb, deletion at the IgH-BCL2 breakpoint (FIG. 14) and a IgH-MYC breakpoint that falls outside the S_{γ3} switch repeat region and exhibits junctional microhomology suggest non-conventional mechanisms underlying these rearrangements (Jager, Bocskor et al., Blood 95(10:3520-3529 (2000); Shou, Martelli et al., PNAS 97(1):228-233 (2000); Corneo, Wendland et al., Nature 449(7161):483-486 (2007); and Yan, Boboila et al., Nature 449(7161)478-482 (2007)). Indeed, subsequent attempts to identify a reciprocal IgH-MYC fusion using J_{H}, D_{H} or S_{H} linear amplification primers were unsuccessful, suggesting either a non-canonical IgH breakpoint or a more complex rearrangement involving a non-IgH partner locus (Changanti, Rao et al., Genes Chromosomes and Cancer 23(4):328-336 (1998)).

A subset of MYC or BCL6 gene rearrangements do not involve IgH but instead the immunoglobulin kappa and lambda light chain loci or various other non-IgH loci (Akasaka, Akasaka et al., Cancer Res. 60(9):2335-2341 (2000); Shou, Martelli et al., PNAS 97(1):228-233 (2000)). To demonstrate that tCGH is capable of detecting non-IgH rearrangements at these loci, linear amplification primers designed to target translocation breakpoint hotspots located near MYC and BCL6 exon 1 were used (Akasaka, Akasaka et al., Cancer Res. 60(9):2335-2341 (2000); Busch, Keller et al., Leukemia 21(8):1739 (2007). On log-ratio plots, these rearrangements (FIG. 10e, 10i) appear similar to the IgH rearrangements described above except for their ‘inverted’ orientation, an anticipated feature of breakpoints identified by tCGH within the same locus as the linear amplification primer (“Gene A” in FIG. 7). These experiments establish that tCGH is capable of detecting MYC and BCL6 rearrangements and identifying breakpoints within these loci even if their rearrangement partners are not known or represented on the array.

### Example 6

### Identification of Novel Chromosomal Duplications and Deletions

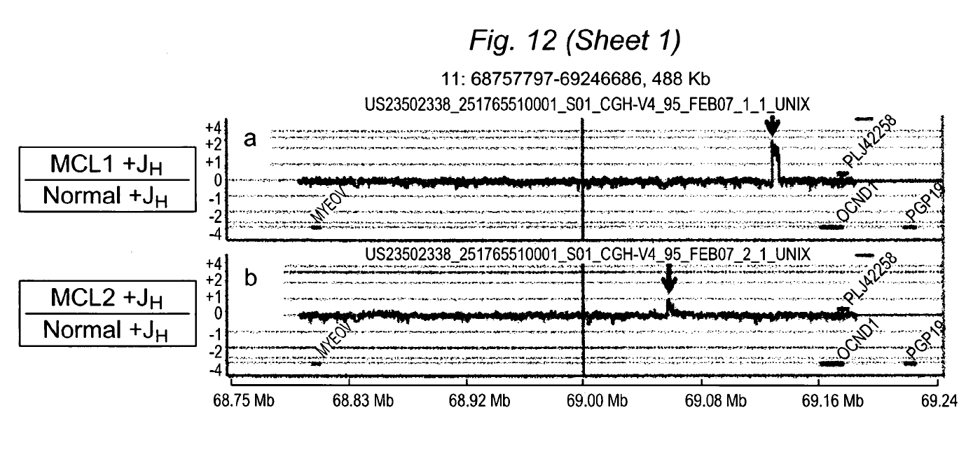

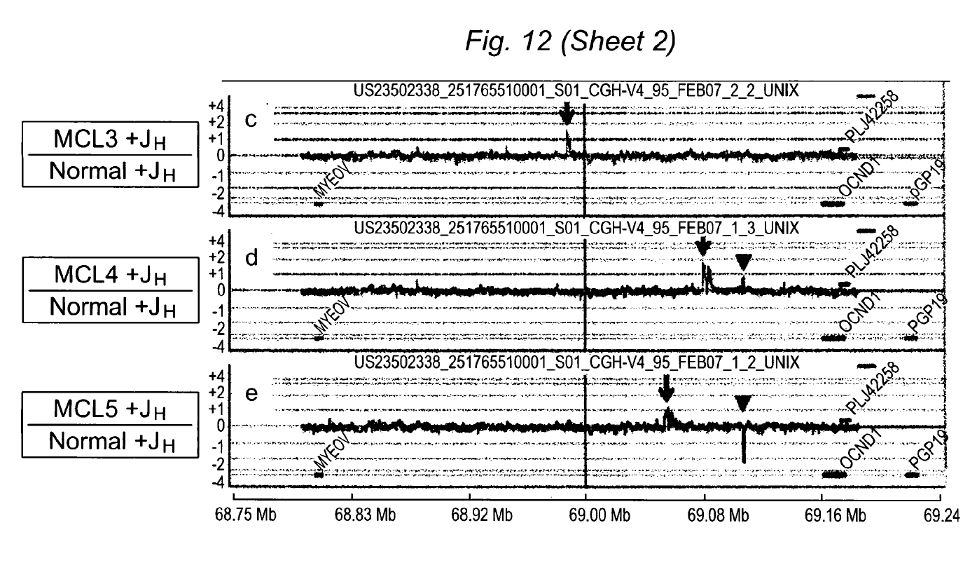

Because tumor and normal genomic DNA are co-hybridized to the same array after linear amplification and labeling (FIG. 7), it was anticipated that tCGH would detect copy number changes in addition to balanced rearrangements. Indeed, several previously unrecognized deletions and duplications at the BCL2 and CCND1 loci in association with IgH translocations at the same loci were identified. In the RL7 lymphoma cell line, for example, which has a known J_{H}-BCL2 translocation, a novel 167 kb interstitial deletion within the large (190 kb) intron of BCL2 was identified (FIG. 11a). A similar 135 kb intronic BCL2 deletion was identified in OCI-Ly8, as was a distinct 6.2 kb deletion at the J_{H}-BCL2 breakpoint junction (FIG. 13). These abnormalities were each confirmed by PCR amplification and sequencing. In both MO2058 and Granta 519, duplications were identified that span the CCND1 gene and extend precisely to the respective J_{H}-CCND1 breakpoint junctions, suggesting that each duplication event occurred on the der(14)t(11;14) chromosome (FIG. 13). A known 1852 nt deletion in the CCND1 3′ untranslated region (Withers, Harvey et al., Mol. Cell. Biol. 11(10):4846-4853 (1991)) also was identified in MO2058 (FIG. 13a). Finally, a novel 562 nt deletion polymorphism was identified and characterized at about 55 kb upstream of the CCND1 gene (FIG. 12b). tCGH detection of copy number changes was independent of the particular linear amplification employed (FIG. 11a-c and FIG. 14). The breakpoints of interstitial deletions also can be mapped by linear amplification across the deletion breakpoints (FIG. 11 panels a, d, e).

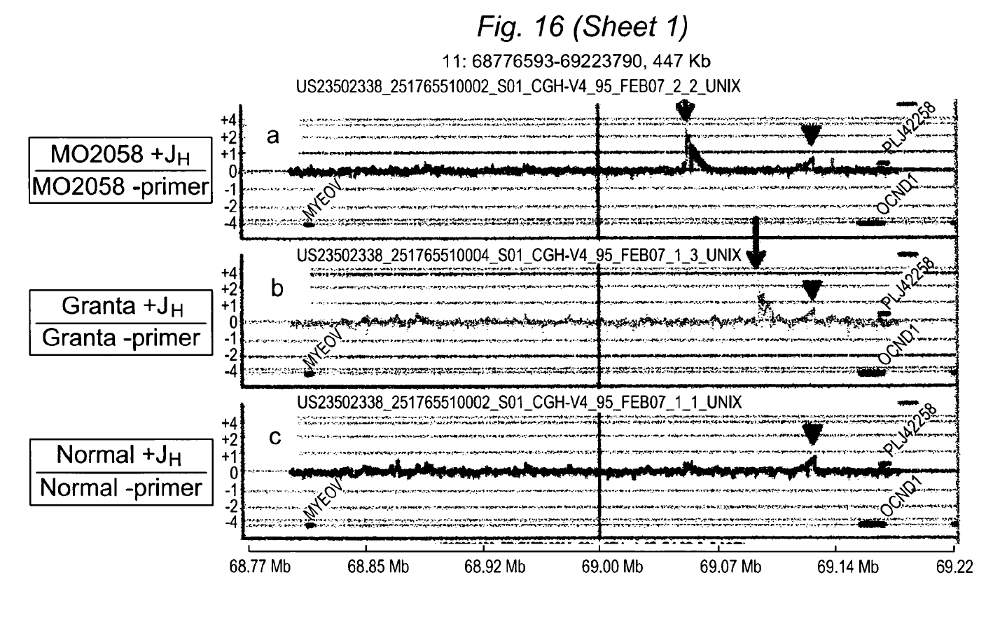

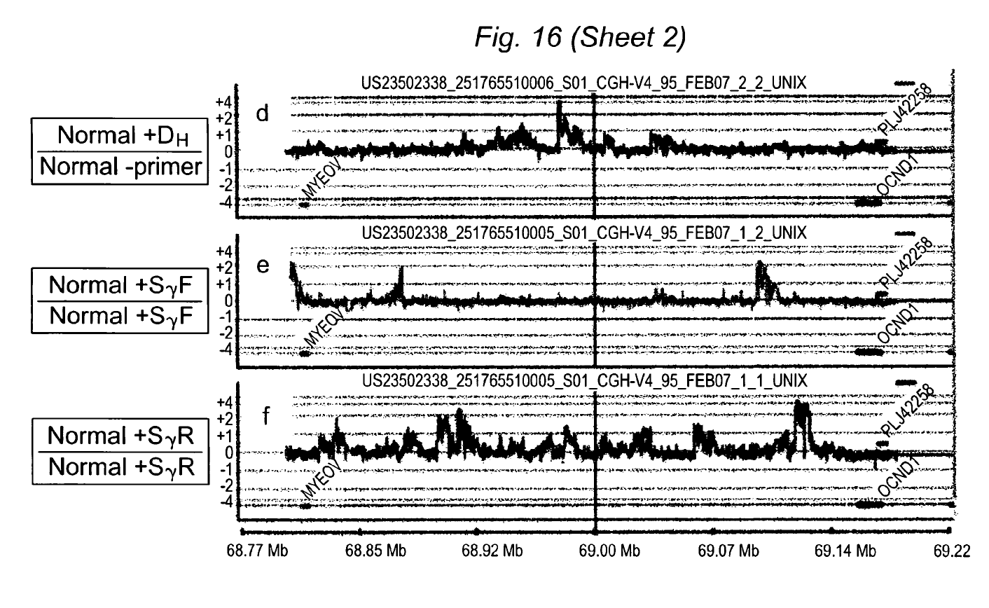

In the tCGH experiments described above, linearly amplified tumor DNA is co-hybridized with similarly amplified normal genomic DNA (FIG. 7). When mock-amplified tumor DNA rather than amplified normal DNA is used as the hybridization control, tCGH would be expected to detect balanced rearrangements but not copy number changes (FIG. 11d and FIGS. 16a and 16b). Under these conditions, however, “off-target” amplification away from expected translocation breakpoints was also observed, even for normal genomic DNA. The number and pattern of off-target amplification signals depends on the linear amplification primer and appears to be more complex for S_{H} primers, which target repetitive switch sequences, but the pattern of off-target signals is remarkably reproducible for any given primer (FIGS. 15 and 16). When tumor and normal DNA are amplified using the same primer and co-hybridized as in conventional tCGH (FIG. 7), the off-target signals for the two samples are perfectly matched and thereby effectively mask one another, revealing only the targeted translocation breakpoint.

The t(11;14) and associated J_{H}-CCND1 fusion are pathognomonic for mantle cell lymphoma (MCL). In ˜40% of MCL cases, CCND1 breakpoints are located within the ˜100 nt major translocation cluster (MTC) (van Dongen, Langerak et al., Leukemia 17(12): 2257-317 (2003)) whereas the other ˜60% of MCL cases have non-MTC breakpoints that are scattered across a ˜400 kb region flanking the MTC (Vaandrager, Schuuring et al., Blood 88(4):1177-1182 (1996)). In contrast to MTC breakpoints, which are readily cloned and analyzed (Wetzel, Le et al., Cancer Res. 61(4):1629-1636 (2001)), non-MTC breakpoints have proven more difficult to clone and only rare examples have been sequenced (Meeker, Grimaldi et al., Blood 74(5):1801-1806 (1989); Meeker, Sellers et al., Leukemia 5(9):733-7 (1991); Willis, Jadayel et al., Blood 90(6):2456-2464 (1997). To determine whether tCGH is capable of identifying novel breakpoints in primary lymphoma samples, we analyzed five primary MCL cases having non-MTC breakpoints. The novel CCND1 breakpoints were each mapped to 100 nt resolution (FIG. 12), enabling rapid confirmation and sequencing of the breakpoint junctions. The breakpoints were scattered across about a 150 kb region centromeric to the CCND1 gene including one located just about 200 bp away from the MTC.

### Example 6

### Establishment and Validation of a tCGH Array for the Detection of Balanced Translocations in Myeloid Leukemias

The present example illustrates the establishment of a tCGH array that is useful for the detection of chromosome abnormalities, such as balanced translocations, chromosomal deletions, chromosomal duplications, and chromosomal inversions, that are common in myeloid leukemias. A microarray was constructed containing 14,262 probe sequences covering eleven genes that are commonly disrupted by chromosomal abnormalities, such as translocations and large deletion, covering over 1.1 Mbp at an average spacing of 83 nt (Table 4). Primer mixes (Table 5) were then used for multiplex linear amplification of chromosomal DNA isolated from various myeloid leukemia cell lines. tCGH analysis was performed on the amplified chromosomal sequences by hybridization to the AML pilot array outlined in Table 4.

TABLE 4
Identification of loci and genomic regions covered
by hybridization probes in the AML pilot array.

| | | | Region | | |
|---|---|---|---|---|---|
| Locus | Genomic Interval (hg17) + 20 kb | Band | Length | # probes | Spacing |
| BCR | chr22: 21,837,106-21,992,698 | 22q11.23 | 155,592 | 2050 | 76 |
| ABL | chr9: 130,608,822-130,802,614 | 9q34.12 | 193,792 | 2279 | 85 |
| ETO (RUNX1T1) | chr8: 93,030,328-93,167,540 | 8q21 | 137,212 | 2021 | 68 |
| AML1 | chr21: 35,118,575-35,163,745 | 21q22.12 | 45,170 | 700 | 65 |
| RARA | chr17: 35,708,972-35,777 420 | 17q21.2 | 68,448 | 880 | 78 |
| PML | chr15: 72,064,067-72,136,162 | 15q22 | 72,095 | 955 | 75 |
| CBFB | chr16: 65,610,551-65,702,457 | 16q22.1 | 91,906 | 973 | 94 |
| MYH11 | chr16: 15,711,505-15,744,067 | 16p13.11 | 32,562 | 358 | 91 |
| MLL | chr11: 117,802,415-117,911,144 | 11q23 | 108,729 | 1374 | 79 |
| AF9 | chr9: 20,324,968-20,457,000 | 9p21.3 | 132,032 | 1997 | 66 |
| IKZF1 (Ikaros) | chr7: 50,111,639-50,257,007 | 7p12.2 | 145,368 | 675 | 215 |
| | AML pilot array total | | 1,182,906 nt | 14,262 | 83 nt |

TABLE 5
Primer mixes for multiplex linear amplification of chromosomal
DNA in tCGH analysis of myeloid leukemias.

| MPM | | | |
|---|---|---|---|
| (myeloid | | SEQ | |
| primer | | ID | |
| mix) | | NO: | Reference |
| BCR-b2-C | CAG ATG CTG ACC AAC TCG TGT | 16 | van Dongen et al., |
| | | | Leukemia. 1999 |
| | | | Dec; 13(12): 1901-28. |
| MYH11-D1 | TCC CTG TGA CGC TCT CAA CT | 17 | van Dongen et al., |
| | | | Leukemia. 1999 |
| MYH11-D2 | CTT GAG CGC CTG CAT GTT | 18 | van Dongen et al., |
| | | | Leukemia. 1999 |
| PML-Cl | TCA AGA TGG AGT CTG AGG | 19 | van Dongen et al., |
| | AGG | | Leukemia. 1999 |
| PML-C2 | AGC GCG ACT ACG AGG AGA T | 20 | van Dongen et al., |
| | | | Leukemia. 1999 |

“821” = (821A6-R + 821A12-R + 821A18-R + 821A24-R)

| | | | |
|---|---|---|---|
| 821A6-R | GTC TTC ACA AAC CCA CCG CAA | 21 | Xiao et al., Leukemia. |
| | GTC G | | 2001 Dec; 15(12): 1906- |
| | | | 13 |
| 821A12-R | GCT GGT CAC AGG TGC CAG | 22 | Xiao et al., Leukemia. |
| | CAG TTG | | 2001 |
| 821A18-R | CTG AGT CTC AGT GCC AGT TTC | 23 | Xiao et al., Leukemia. |
| | CCA GGA | | 2001 |
| 821A24-R | CTG ATG AAA CAG CTG GAG | 24 | Xiao et al., Leukemia. |
| | GCA GAG GG | | 2001 |

“P1/P7” = (MLL-P1LR-F + MLL-P7LR-F)

| | | | |
|---|---|---|---|
| MLL-P1LR-F | CCG CCT CAG CCA CCT ACT ACA | 25 | Langer et al., Genes |
| | GGA CCG | | Chromosomes Cancer. |
| | | | 2003 Apr; 36(4): 393- |
| | | | 401 |
| MLL-P7LR-F | CAA CCC GAA AGT CCA TCT ATA | 26 | Langer et al., Genes |
| | GGG AGC ATG GG | | Chromosomes Cancer. |
| | | | 2003 |

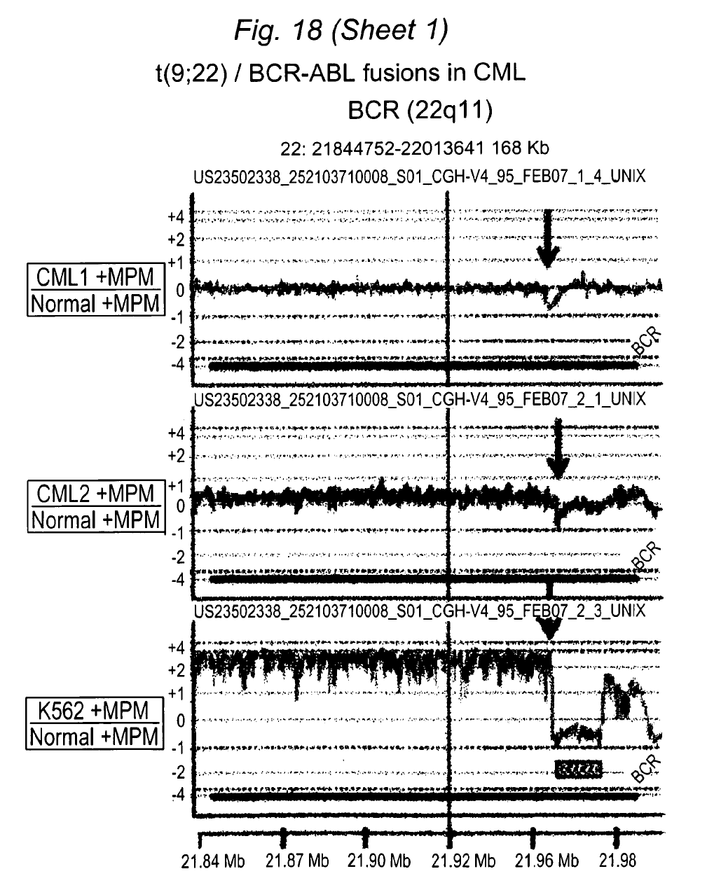

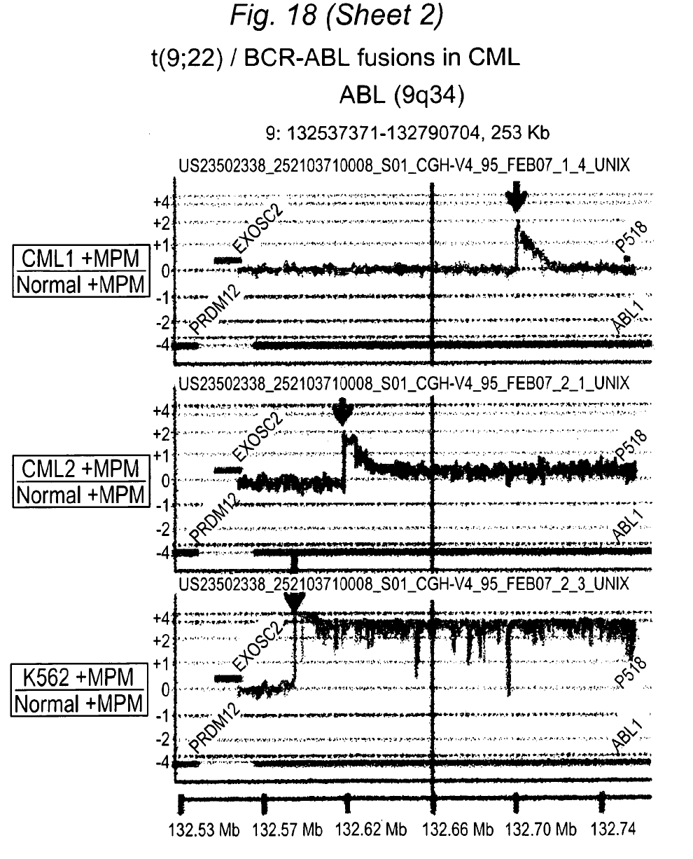

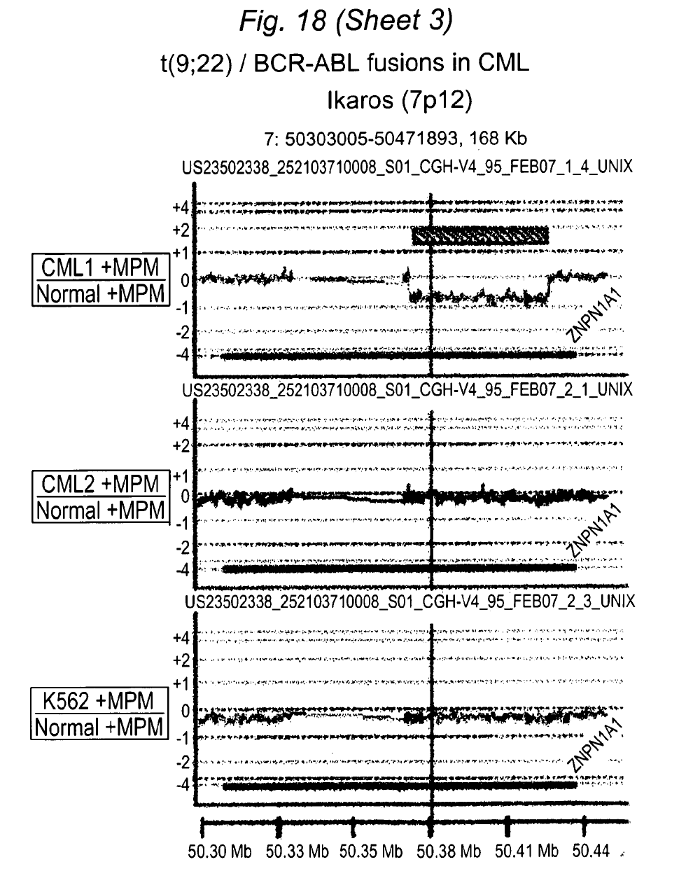

FIG. 18 shows the results of multiplex tCGH analysis, using the AML pilot array, of three chronic myeloid leukemia cell lines, CML1, CML2, and K562, with the MPM primer set. The translocation break points are clearly seen in the analysis. Also illuminated by the analysis is a large chromosomal deletion in the ikaros gene of the CML1 leukemia cell line. This example demonstrates that multiplex tCGH analysis can simultaneously determine the genotype of balanced BCR-ABL translocations associated with chronic myeloid leukemia, as well associated chromosomal deletions and amplifications.

FIG. 19 shows the results of multiplex tCGH analysis, using the AML pilot array, of two acute promyelocytic leukemia cell lines (APL1 and APL2) characterized by PML-RARA balanced translocations t(15;21) having different translocation break points in the RARA gene, and two acute myelomonocytic leukemia/eosinophilia cell lines (M4Eo1 and M4Eo2) characterized by MYH11-CBFB fusions caused by a chromosomal inversion inv(16)/t(16:16), using the myeloid primer mix (MPM) and AML pilot array. As can be seen in the present example, multiplex tCGH analysis can simultaneously determine the genotypes of various chromosomal abnormalities, such as balanced translocations and chromosomal inversions.

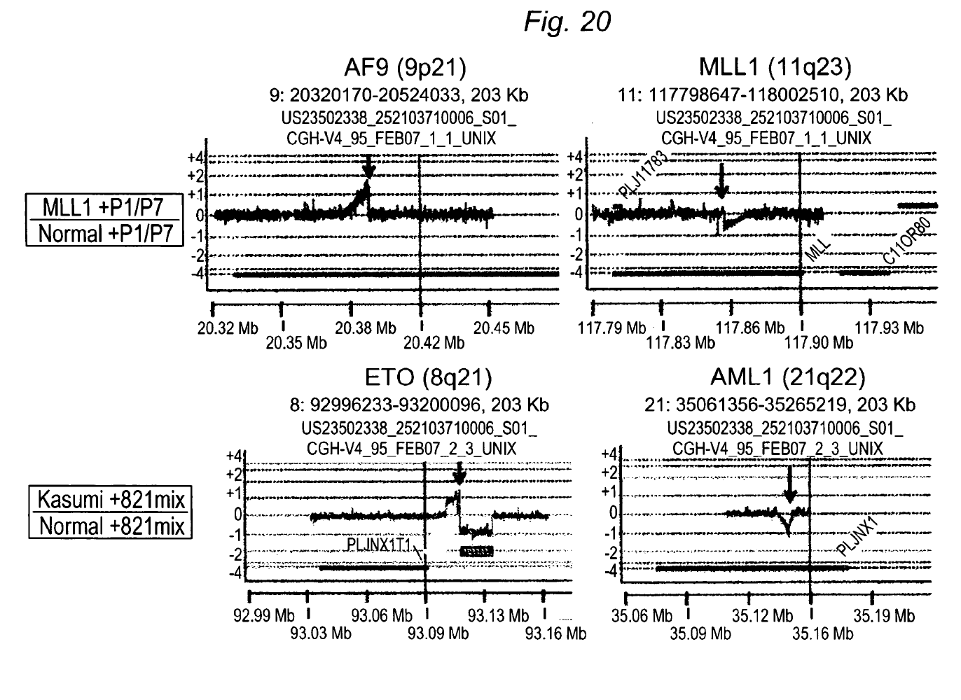

FIG. 20 shows the results of multiplex tCGH analysis, using the AML pilot array, of an MLL leukemia cell line characterized by an AF9-MLL balanced translocation t(9;11) using the P1/P7 primer mix (MPM) and AML pilot array, and of a Kasumi Acute Myeloid Leukemia cell line characterized by an ETO-AML1 balanced translocations t(8;21), using the 821 primer mix (MPM) and AML pilot array. The present example demonstrates that multiplex tCGH is amenable for use in genotyping a wide range of chromosomal abnormalities, including balanced translocations, chromosomal deletions, chromosomal duplications, chromosomal inversions.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A method of determining a chromosomal rearrangement at known genomic locus in a test sample, the method comprising:
(a) isolating a first genomic DNA from cells of a test sample and a second genomic DNA from cells of a reference sample;
(b) performing linear amplification and labeling of the first genomic DNA sample using a primer specific for a known DNA sequence within the known genomic locus to generate an amplified test DNA product comprising a first detectable label; and performing linear amplification and labeling of the second genomic DNA sample using the primer specific for a known DNA sequence within known genomic locus to generate an amplified reference DNA product comprising a second detectable label;
(c) hybridizing the amplified test and reference DNA products to a DNA microarray comprising genomic DNA sequences; and
(d) comparing the pattern and extent of hybridization of the test amplified DNA product with the reference amplified DNA product to the DNA microarray;
wherein excess hybridization of the linear amplified test sample DNA product over the linear amplified reference sample DNA product to a DNA microarray element distinct from that of the known genomic locus is indicative of a rearrangement of the known genomic locus with a second genomic locus in the cell.

2. The method of claim 1, wherein the rearrangement is a chromosomal translocation.

3. The method of claim 1, wherein the first and second detectable labels are incorporated during amplification.

4. The method of claim 1, wherein the first and second detectable labels are incorporated after amplification.

5. The method of claim 1, wherein the first and second detectable label are fluorescent labels.

6. The method of claim 5, wherein the fluorescent labels are Cy3 and Cy5.

7. The method of claim 1, wherein the DNA microarray is a tiling density DNA microarray.

8. The method of claim 1, wherein the known genomic locus corresponds to an immunoglobulin gene.

9. The method of claim 1, wherein the cell of the test sample is a tumor cell and the reference cell is a normal cell.

10. The method of claim 9, wherein the tumor cell is a lymphoma or leukemia.

11. A method of identifying a chromosomal rearrangement partner of a known genetic locus in a test sample, the method comprising:
(a) isolating a first genomic DNA from cells of a test sample and a second genomic DNA from cells of a reference sample;
(b) performing linear amplification and labeling of the first genomic DNA sample using a primer specific for a known DNA sequence within known genomic locus to generate an amplified test DNA product comprising a first detectable label; and performing linear amplification and labeling of the second genomic DNA sample using the primer specific for a known DNA sequence within known genomic locus to generate an amplified reference DNA product comprising a second detectable label;
(c) hybridizing the labeled and amplified test and reference DNA products to a DNA microarray comprising genomic DNA sequences; and
(d) comparing the pattern and extent of hybridization of the test amplified DNA product with the reference amplified DNA product to the DNA microarray;
wherein excess hybridization of the linear amplified test sample DNA product over the linear amplified reference sample DNA product to a DNA microarray element distinct from that of the known genomic locus identifies the element of the DNA microarray as a rearrangement partner of the known genomic locus.

12. (canceled)

13. The method of claim 11, further comprising the step of determining the last element in a series of elements corresponding to the linear sequence of the known genomic locus that hybridizes to the amplified DNA product, thereby identifying the approximate location of the rearrangement breakpoint of the known genomic locus.

14. The method of claim 11, further comprising the step of determining the first element in a series of elements, corresponding to the linear sequence of a second genomic locus distinct from that of the known genomic locus, that hybridizes to the amplified DNA product, thereby identifying the approximate location of the rearrangement breakpoint of the rearrangement partner.

15. (canceled)

16. (canceled)

17. (canceled)

18. (canceled)

19. (canceled)

20. (canceled)

21. (canceled)

22. (canceled)

23. A method of simultaneously determining chromosomal rearrangements, including chromosomal translocations, at a known genomic locus of a test sample, the method comprising:
(a) isolating a first genomic DNA from cells of a test sample and a second genomic DNA from cells of a reference sample;
(b) performing linear amplification of the first genomic DNA using a primer specific for a known DNA sequence within known genomic locus to generate a mixture of test genomic DNA and primer specific, amplified test DNA product; and performing linear amplification of the second genomic DNA using the same specific primer for a known DNA sequence within known genomic locus sample to generate a mixture of reference genomic DNA and primer specific, amplified reference DNA product;
(c) further amplifying and labeling the test and reference sample mixtures via oligonucleotide primed, polymerase mediated extension;
(d) hybridizing the labeled and amplified test and reference DNA product to a DNA microarray comprising genomic DNA sequences; and
(e) comparing the pattern and extent of hybridization of the test amplified DNA product with the reference amplified DNA product to the DNA microarray;
wherein
(i) a greater extent of hybridization of the test amplified DNA product to an element of the DNA microarray as compared to the extent of hybridization of the reference amplified DNA product to the element of the DNA microarray, when both hybridize to the element, indicates an amplification of the DNA sequence represented by the element of the microarray in the test sample;
(ii) hybridization of the reference amplified DNA product to an element of the DNA microarray in excess of hybridization of the test amplified DNA product to the element of the DNA microarray indicates a deletion of the DNA sequence represented by the element of the microarray in the test sample; and
(iii) hybridization of the test amplified DNA product to a DNA array element distinct from that of the known genomic locus in excess of hybridization of the reference amplified DNA product to the DNA array element is indicative of a translocation of the known genomic locus with a second genomic locus in the cell.

24. (canceled)

25. (canceled)

26. (canceled)

27. (canceled)

28. (canceled)

29. (canceled)

30. (canceled)

31. (canceled)

32. The method of claim 23, wherein the test and reference samples comprise the same genomic DNA, and the test sample, but not the reference sample, is subjected to the linear amplification step of part (b).

33. The method of claim 1, wherein the first and second detectable labels are the same and the hybridizing of the amplified test and reference DNA products is to separate but identical microarrays or sequentially to the same microarray.

34. A method of determining a chromosomal rearrangement in a test sample, the method comprising:
(a) isolating a first genomic DNA from cells of a test sample and a second genomic DNA from cells of a reference sample;
(b) performing linear amplification of the first genomic DNA sample using a primer specific for a known DNA sequence within a known genomic locus to generate an amplified test DNA product (T+); performing linear amplification and labeling of the second genomic DNA sample using the primer specific for a known DNA sequence within the known genomic locus to generate an amplified reference DNA product (N+); performing a mock linear amplification of the first genomic DNA sample by omitting the primer specific for a known DNA sequence within a known genomic locus to generate a mock-amplified test DNA product (T−); performing a mock linear amplification of the second genomic DNA sample by omitting the primer specific for a known DNA sequence within a known genomic locus to generate a mock-amplified reference DNA product (N−);
(c) labeling each of T+, N+, T−, and N− with a different detectable label by primer extension using random primers;
(d) co-hybridizing T+ and N+ to a first DNA microarray comprising genomic DNA sequences;
(e) co-hybridizing T− and N− to a second DNA microarray comprising genomic DNA sequences;
(f) comparing the pattern and extent of hybridization signal on the first DNA microarray with the pattern and extent of hybridization signal on the second DNA microarray;
wherein a right triangular pattern of hybridization signal on a scatter plot of hybridization signal plotted against chromosomal position from the first microarray in the absence of a similar pattern from the second microarray is indicative of a chromosomal translocation, with the chromosomal position of the vertical leg marking a chromosomal translocation breakpoint; and
wherein a rectangular pattern of hybridization signal on a scatter plot of hybridization signal plotted against chromosomal position at the same position from the first microarray and the second microarray is indicative of a chromosomal duplication or deletion with the chromosomal positions of the vertical legs marking the two end points of a duplicated or deleted genomic region,
thereby providing a determination of a chromosomal rearrangement in the test sample.

35. The method of claim 34, wherein the mock amplification to generate T− and N− comprises no amplification.

36. (canceled)

37. (canceled)

38. (canceled)

39. (canceled)

40. (canceled)

41. (canceled)

42. A method of determining a chromosomal rearrangement in a test sample, the method comprising:
(a) isolating a first genomic DNA from cells of a test sample and a second genomic DNA from cells of a reference sample;
(b) performing linear amplification of the first genomic DNA sample using a primer specific for a known DNA sequence within a known genomic locus to generate an amplified test DNA product (T+); performing linear amplification and labeling of the second genomic DNA sample using the primer specific for a known DNA sequence within the known genomic locus to generate an amplified reference DNA product (N+); performing a mock linear amplification of the first genomic DNA sample by omitting the primer specific for a known DNA sequence within a known genomic locus to generate a mock-amplified test DNA product (T−); performing a mock linear amplification of the second genomic DNA sample by omitting the primer specific for a known DNA sequence within a known genomic locus to generate a mock-amplified reference DNA product (N−);
(c) labeling each of T+, N+, T−, and N− with a different detectable label by primer extension using random primers;
(d) co-hybridizing T+ and T− to a first DNA microarray comprising genomic DNA sequences;
(e) co-hybridizing N+ and N− to a second DNA microarray comprising genomic DNA sequences;
(f) comparing the pattern and extent of hybridization signal on the first DNA microarray with the pattern and extent of hybridization signal on the second DNA microarray;
wherein a right triangular pattern of hybridization signal on a scatter plot of hybridization signal plotted against chromosomal position from the first microarray in the absence of a similar pattern from the second microarray is indicative of a chromosomal translocation with the chromosomal position of the vertical leg marking a chromosomal translocation breakpoint; and
wherein patterns of hybridization signal on a scatter plot of hybridization signal plotted against chromosomal position common to both the first microarray and the second microarray indicate pseudo-breakpoints,
thereby providing a determination of a chromosomal rearrangement in the test sample.

43. (canceled)

44. (canceled)

45. (canceled)

46. (canceled)

47. (canceled)

48. (canceled)

49. A method of diagnosing a disease in a subject, said disease resulting from a chromosomal rearrangement, the method comprising the steps of:
(a) obtaining a biological sample from the subject;
(b) isolating a first genomic DNA from cells of the biological sample and a second genomic DNA from cells of a reference sample;
(c) performing linear amplification and labeling of the first genomic DNA sample using a primer specific for a known DNA sequence within a known genomic locus associated with the disease to generate an amplified test DNA product comprising a first detectable label; and performing linear amplification and labeling of the second genomic DNA sample using the primer specific for a known DNA sequence within the known genomic locus to generate an amplified reference DNA product comprising a second detectable label;
(d) hybridizing the amplified test and reference DNA products to a DNA microarray comprising genomic DNA sequences; and
(e) comparing the pattern and extent of hybridization of the test amplified DNA product with the reference amplified DNA product to the DNA microarray;
wherein excess hybridization of the linear amplified test sample DNA product over the linear amplified reference sample DNA product to a DNA microarray element distinct from that of the known genomic locus identifies the element of the DNA microarray as a rearrangement partner of the known genomic locus and the identity of the rearrangement partner provides a diagnosis of the disease in the subject.

50. (canceled)

51. The method of claim 49, wherein the disease is a cancer.

52. The method of claim 51, wherein the cancer is lymphoma or leukemia.

53. (canceled)

54. The method of claim 49, wherein the rearrangement partner is MYC.

55. The method of claim 52, wherein the lymphoma is Burkitt's lymphoma.

56. A method of detecting a chromosomal rearrangement, the method comprising the steps of:
(a) amplifying a target genomic locus;
(b) hybridizing said amplified product to a nucleic acid array; and
(c) comparing said hybridization pattern to a reference,
wherein said amplification is linear amplification, and wherein differential hybridization of the amplified genomic locus as compared to the reference indicates the presence of a genomic rearrangement.

57. The method of claim 56, wherein said chromosomal rearrangement is a balanced chromosomal rearrangement.

58. A method of detecting a balanced chromosomal translocation, the method comprising the steps of:
(a) amplifying a target genomic locus;
(b) hybridizing said amplified product to a nucleic acid array; and
(c) comparing said hybridization pattern to a reference,
wherein said amplification is linear amplification, and wherein the presence of a right triangular hybridization pattern indicates the presence of a balanced chromosomal translocation.

59. The method of claim 56, wherein said method comprises multiplex linear amplification.

60. The method of claim 56, wherein more than one genomic loci is surveyed in a single assay.

61. The method of claim 56, wherein a plurality of linear amplification primers is used.

62. The method of claim 61, wherein said plurality of primers comprise primers for the amplification of loci implicated in balanced translocations associated with a disease.

63. (canceled)

64. (canceled)

65. The method of claim 61, wherein said plurality of primers is selected from the MPM mix, the 821 mix, the P1/P7 mix, and a plurality of D_{H} primers.

66. The method of claim 56, wherein said nucleic acid array is a high density tiled array comprising probes to a plurality of genomic loci.

67. The method of claim 66, wherein at least one of said plurality of genomic loci is a locus associated with a disease.

68. (canceled)

69. (canceled)

70. The method of claim 56, wherein said array is an AML pilot array.

71. The method of claim 56, wherein the method further comprises the detection of a second chromosomal rearrangement.

72. The method of claim 71, wherein said second chromosomal rearrangement is selected from the group consisting of a duplication, an amplification, a deletion, an inversion, a balanced translocation, and an unbalanced translocation.

73. The method of claim 56, wherein said method comprises the detection of both partner loci of a translocation.

74. A method of diagnosing or providing a prognosis for a lymphoma in an individual, the method comprising the detection, in a biological sample from said individual, of an IgH translocation, wherein the IgH partner chromosomal breakpoint is selected from those listed in Table 2.

75. The method of claim 74, wherein said translocation is a balanced translocation.

76. The method of claim 74, wherein the detection is by PCR, sequencing, mass spectrometry, hybridization, or tCGH analysis.

77. A kit for use in the detection of a balanced chromosomal translocation, the kit comprising:
(a) a primer for the linear amplification of a genomic locus implicated in a translocation; and
(b) an array for the detection of product amplified by said primer.

78. The kit of claim 77, wherein said kit comprises a plurality of primers for the linear amplification of loci implicated in translocations.

79. (canceled)

80. (canceled)

81. (canceled)

82. An array for tCGH analysis of a chromosomal rearrangement associated with a disease.

83. The array of claim 82, wherein said array comprises a plurality of probes specific for at least two loci, wherein the loci are implicated in a chromosomal rearrangement associated with a disease.

84. The array of claim 82, wherein at least one rearrangement associated with a disease is a balanced translocation.

85. (canceled)

86. The array of claim 82, wherein at least one locus detected by the array is selected from those found in Table 2 and Table 4.

87. (canceled)

88. (canceled)

89. An oligonucleotide selection algorithm as used in the computer program Tile.


SEQUENCE LISTING (65 sequences)

SEQ ID NO: 1 - DNA, 22 bp, Artificial Sequence
  synthetic amplification primer JH
cttacctgaggagacggtgacc

SEQ ID NO: 2 - DNA, 15 bp, Artificial Sequence
  synthetic amplification primer SpF
gcycagcycagcyca

SEQ ID NO: 3 - DNA, 15 bp, Artificial Sequence
  synthetic amplification primer SpR
grgctgrgctgrgct

SEQ ID NO: 4 - DNA, 30 bp, Artificial Sequence
  synthetic amplification primer SgammaF
ggctgctctgccctggtcccctgagctcca

SEQ ID NO: 5 - DNA, 30 bp, Artificial Sequence
  synthetic amplification primer SgammaR
tggagctcaggggaccagggcagagcagcc

SEQ ID NO: 6 - DNA, 18 bp, Artificial Sequence
  synthetic amplification primer DH1
ggcggaatgtgtgcaggc

SEQ ID NO: 7 - DNA, 21 bp, Artificial Sequence
  synthetic amplification primer DH2
gcactgggctcagagtcctct

SEQ ID NO: 8 - DNA, 20 bp, Artificial Sequence
  synthetic amplification primer DH3
gtggccctgggaatataaaa

SEQ ID NO: 9 - DNA, 19 bp, Artificial Sequence
  synthetic amplification primer DH4
agatccccaggacgcagca

SEQ ID NO: 10 - DNA, 20 bp, Artificial Sequence
  synthetic amplification primer DH5
cagggggacactgtgcatgt

SEQ ID NO: 11 - DNA, 19 bp, Artificial Sequence
  synthetic amplification primer DH6
tgaccccagcaagggaagg

SEQ ID NO: 12 - DNA, 18 bp, Artificial Sequence
  synthetic amplification primer DH7
cacaggccccctaccagc

SEQ ID NO: 13 - DNA, 35 bp, Artificial Sequence
  synthetic amplification primer Bcl6R
agaattccagaggccgagctttgctacagcgaagg

SEQ ID NO: 14 - DNA, 22 bp, Artificial Sequence
  synthetic amplification primer Bcl2F
ctcatgccccatattcattcaa

SEQ ID NO: 15 - DNA, 20 bp, Artificial Sequence
  synthetic amplification primer MycF
ggtcggacattcctgcttta

SEQ ID NO: 16 - DNA, 21 bp, Artificial Sequence
  synthetic primer BCR-b2-C for primer mix MPM (myeloid primer mix) used for multiplex linear amplification of chromosomal DNA
cagatgctgaccaactcgtgt

SEQ ID NO: 17 - DNA, 20 bp, Artificial Sequence
  synthetic primer MYH11-D1 for primer mix MPM (myeloid primer mix) used for multiplex linear amplification of chromosomal DNA
tccctgtgacgctctcaact

SEQ ID NO: 18 - DNA, 18 bp, Artificial Sequence
  synthetic primer MYH11-D2 for primer mix MPM (myeloid primer mix) used for multiplex linear amplification of chromosomal DNA
cttgagcgcctgcatgtt

SEQ ID NO: 19 - DNA, 21 bp, Artificial Sequence
  synthetic primer PML-C1 for primer mix MPM (myeloid primer mix) used for multiplex linear amplification of chromosomal DNA
tcaagatggagtctgaggagg

SEQ ID NO: 20 - DNA, 19 bp, Artificial Sequence
  synthetic primer PML-C2 for primer mix MPM (myeloid primer mix) used for multiplex linear amplification of chromosomal DNA
agcgcgactacgaggagat

SEQ ID NO: 21 - DNA, 25 bp, Artificial Sequence
  synthetic primer 821A6-R for primer mix “821” used for multiplex linear amplification of chromosomal DNA
gtcttcacaaacccaccgcaagtcg

SEQ ID NO: 22 - DNA, 24 bp, Artificial Sequence
  synthetic primer 821A12-R for primer mix “821” used for multiplex linear amplification of chromosomal DNA
gctggtcacaggtgccagcagttg

SEQ ID NO: 23 - DNA, 27 bp, Artificial Sequence
  synthetic primer 821A18-R for primer mix “821” used for multiplex linear amplification of chromosomal DNA
ctgagtctcagtgccagtttcccagga

SEQ ID NO: 24 - DNA, 26 bp, Artificial Sequence
  synthetic primer 821A24-R for primer mix “821” used for multiplex linear amplification of chromosomal DNA
ctgatgaaacagctggaggcagaggg

SEQ ID NO: 25 - DNA, 27 bp, Artificial Sequence
  synthetic primer MLL-P1LR-F for primer mix “P1/P7” used for multiplex linear amplification of chromosomal DNA
ccgcctcagccacctactacaggaccg

SEQ ID NO: 26 - DNA, 32 bp, Artificial Sequence
  synthetic primer MLL-P7LR-F for primer mix “P1/P7” used for multiplex linear amplification of chromosomal DNA
caacccgaaagtccatctatagggagcatggg

SEQ ID NO: 27 - DNA, 20 bp, Artificial Sequence
  synthetic PCR/sequencing primer IgH
gacccagcacccttatttcc

SEQ ID NO: 28 - DNA, 22 bp, Artificial Sequence
  synthetic PCR/sequencing primer CCND1
gatcacagtctttgctgcctgt

SEQ ID NO: 29 - DNA, 251 bp, Artificial Sequence
  synthetic breakpoint sequence for MCL1 J-H-CCND1 fusion
cagttttagagttgtttgtggcaggaaagttacttttggccagaattggaagttggaaggtgtgcagctattgctatagcaaatgtgttctccatcctgatcagtaaagaggataaaaagcaatttatcattagataggaaggatattcacaatctcactccagatctatgttataataactcctgttctccaaagaatataggttgactactggggccagggaaccctggtcaccgtctcctcaggtaag

SEQ ID NO: 30 - DNA, 22 bp, Artificial Sequence
  synthetic PCR/sequencing primer IgH
ccaggctcagttactccatcag

SEQ ID NO: 31 - DNA, 23 bp, Artificial Sequence
  synthetic PCR/sequencing primer CCND1
cactctggaacattcttgcattg

SEQ ID NO: 32 - DNA, 634 bp, Artificial Sequence
  synthetic breakpoint sequence for MCL2 J-H-CCND1 fusion
tgtaatccccagcactttgggaggctgatacgggagaatcacttagccccagagaagttccaagaacagcctgggcatcatacatagcgagactcgttctctaaaaaatacaaaaaaattagctgggtgtggtggcacgtgcctgtagtcacagctacttgggaggctgtgatgggaggatcactggagcccaggaactccaggctgtagtgaactatgatcatgccactgctccagcctgggtgacagtgtgagaccctgtctctgataataatcataatattttattagtagagtcgttttttctttttcatttctttttaatttaatgttttgtacggacaagttttcgctattttgcccaggctagtcttgaactcctggcctcaaccgatcctcctgcctcagcctcccaaagcactgggattacaggcaagaaccaccgcacccggcccaaacattttcattttttatatttcaagtactttgattaattattgtgcaagtttcttgtgcaaagcttagaagaagaggtcttacagaattttttgcggtttttaagcaattacaccatataaaactactactactacggtatggacgtctggggccaagggaccacggtcaccgtctc

SEQ ID NO: 33 - DNA, 21 bp, Artificial Sequence
  synthetic PCR/sequencing primer IgH
agaggctcccagatcctcaag

SEQ ID NO: 34 - DNA, 20 bp, Artificial Sequence
  synthetic PCR/sequencing primer CCND1
aacacagtgccatgaaacca

SEQ ID NO: 35 - DNA, 578 bp, Artificial Sequence
  synthetic breakpoint sequence for MCL3 J-H-CCND1 fusion
aacactagatctggaaaatagggtttcatggcccagagtttggggaaccgggcacgagagctgagtcagcatctttggctgtgaaaaatctctgcttaatgttggctgccacgggctccacagcctctttgccatggcatatcttctggcgtgccactgactcacaccatcatgaaatgagcgccatgcagaacacaatcttggggaaaagcttatctaaggaataaaattacaggtccagatcacttatataactggcgcttagaagaagagcccacccacattattttttgaggacccccatggctgatggtgaacccctgctctgaggtgggtggcttcccttcagccccacggcttgttggggggcaccctccacccagctgtcgccttggggtaaggctccccacggagccacccgaaatgaatctggttcgacccctggggccagggaaccctggtcaccgtctcctcaggtgagtcctcaccaccccctctctgagtccacttagggagactcagcttgccagggtctcagggtcagagtcttggaggcattttggaggtcaggaaagaaa

SEQ ID NO: 36 - DNA, 22 bp, Artificial Sequence
  synthetic PCR/sequencing primer IgH
ccaggctcagttactccatcag

SEQ ID NO: 37 - DNA, 19 bp, Artificial Sequence
  synthetic PCR/sequencing primer CCND1
tgtgaccacttcctgacca

SEQ ID NO: 38 - DNA, 626 bp, Artificial Sequence
  synthetic breakpoint sequence for MCL4 J-H-CCND1 fusion
ctttaggtacacggatggaggtctgctggccgaggctggcatttgcacacttcattgcagcactggagaactgagtcttcttttactcaatttttcatagaaataggcacattccccatcccttcccctcgcccccacaacccctgacgctcagcatccagggctgatctgagagggacccgggaggcagagaaaccccagagccgtcatttcccagatgtggcattatgtgtgagcctaggtttgtgttcttttaacggcaccacataaaccccagtcctccaaactggttcgacccctggggccagggaaccctggtcaccgtctcctcaggtgagtcctcaccaccccctctctgagtccacttagggagactcagcttgccagggtctcagggtcagagtcttggaggcattttggaggtcaggaaagaaagccggggagagggacccttcgaatgggaacccagcctgtcctccccaagtccggccacagatgtcggcagctggggggctccttcggctggtctggggtgacctctctccgcttcacctggagcattctcaggggctgtcgtgatgattgcgtggtgggactctgtcccgctccaaggcacccgctctctg

SEQ ID NO: 39 - DNA, 20 bp, Artificial Sequence
  synthetic PCR/sequencing primer IgH
gacccagcacccttatttcc

SEQ ID NO: 40 - DNA, 23 bp, Artificial Sequence
  synthetic PCR/sequencing primer CCND1
accacacctggccttctattgta

SEQ ID NO: 41 - DNA, 310 bp, Artificial Sequence
  synthetic breakpoint sequence for MCL5 J-H-CCND1 fusion
tgtctaatgccctgtatccccattttaacatcatacaaagagtttcactgccctaaaaatctgtctccacctgttcacccctctctccaaattcctggcaaccactgattgttttactttctctgtagttttgtcctttctagaatgtcaaagagttggactcatacggtaacggagagaccagcatacatgactactggggccagggaaccctggtcaccgtctcctcaggtgagtcctcacaacctctctcctgctttaactctgaagggttttgctgcatttttggggggaaataaggggctgggtc

SEQ ID NO: 42 - DNA, 22 bp, Artificial Sequence
  synthetic PCR/sequencing primer IgH
cttacctgaggagacggtgacc

SEQ ID NO: 43 - DNA, 31 bp, Artificial Sequence
  synthetic PCR/sequencing primer BCL2
cgggaattctttgacctttagagagttgctt

SEQ ID NO: 44 - DNA, 416 bp, Artificial Sequence
  synthetic breakpoint sequence for FCL J-H-BCL2 fusion
ttttccaaggcatcggaaatccacagaggctcccagatcctcaaggcaccccagtgcccgtcccctcctggccagtccgcccaggtcccctcggaacatgccccgaggaccaacctgcaatgctcaggaaaccccacaggcagtagcagaaaacaaaggccctagagtggccattcttacctgaggagacggtgaccgtggtcccttggccccagacgtccatcaccgggccccgccggggaggtctggcttcataccacaggtttcctgctttcttggtggagcgtaagcaccactgcatttcaggaagaccctgaaggacagccatgagaaagcccccgcggaaggagggcaggagggctctgggtgggtctgtgttgaaacaggccacgtaaagcaactctctaaaggtcaaa

SEQ ID NO: 45 - DNA, 22 bp, Artificial Sequence
  synthetic PCR/sequencing primer IgH
ccaggctcagttactccatcag

SEQ ID NO: 46 - DNA, 30 bp, Artificial Sequence
  synthetic PCR/sequencing primer BCL2
cgggaattctcagtctctggggaggagtgg

SEQ ID NO: 47 - DNA, 620 bp, Artificial Sequence
  synthetic breakpoint sequence for DHL16 J-H-BCL2 fusion
acaggcagtagcagaaaacaaaggccctagagtggccattcttacctgaggagacggtgaccgtggtcccttggccccagacgtccatattaatatttggcgagacagagaataccacaaagaagtggatagatggcagatgacacatgcgagacccaaagtgctaatttcctgaatatacaaagagctcttacaagttaatcaaaagacaaataactcaatgaagtaatgggctcttgcctatgttatgaatattttcatctggtatgacatttactctttgatttcattatttttgtgattttcataggcttgtattttatgtagtcaaatctctatgtctttctatcacttttgtgtttagaaggacgttcctccttctgaagtacacatatttgattttggatttgagatggcattcatttctgatccatctataaagtttttggtgtgtggttaaaggtaaaaatagactcacattttcctaaataattagccagttgttccagctccatttattgagtggtccttcctttcccaactgatgcgcggtgtaacctttatcatatattaaatactcatgtgtgctaaaaataaaaagtcatttcagttgagtgctg

SEQ ID NO: 48 - DNA, 22 bp, Artificial Sequence
  synthetic PCR/sequencing primer IgH
ccaggctcagttactccatcag

SEQ ID NO: 49 - DNA, 31 bp, Artificial Sequence; identical to SEQ ID NO: 43
  synthetic PCR/sequencing primer BCL2

SEQ ID NO: 50 - DNA, 410 bp, Artificial Sequence
  synthetic breakpoint sequence for OCI-Ly8 J-H-BCL2 fusion
ttttccaaggcatcggaaatccacagaggctcccagatcctcaaggcaccccagtgcccgtcccctcctggccagtccgcccaggtcccctcggaacatgccccgaggaccaacctgcaatgctcaggaaaccccacaggcagtagcagaaaacaaaggccctagagtggccattcttacctgaggagacggtgaccgtggtcccttggccccagacgtccataccgtattttcatcccattcgcacacagggggtaacggggcgccgggtaagcaccactgcatttcaggaagaccctgaaggacagccatgagaaagcccccgcggaaggagggcaggagggctctgggtgggtctgtgttgaaacaggccacgtaaagcaactctctaaaggtcaaagaattcccga

SEQ ID NO: 51 - DNA, 20 bp, Artificial Sequence
  synthetic PCR/sequencing primer BCL2
ctggagcacttcaacagcag

SEQ ID NO: 52 - DNA, 20 bp, Artificial Sequence
  synthetic PCR/sequencing primer IgH
gtggccctgggaatataaaa

SEQ ID NO: 53 - DNA, 631 bp, Artificial Sequence
  synthetic breakpoint sequence for OCI-Ly8 D-H-BCL2 fusion
atttgtgggcacttatgaacccgaaaggacatggccatggggtgggtagggacatagggacagatgccagcctgaggtggagcctcaggacacaggtgggcacggacactatccacataagcgagggatagacccgagtgtccccacagcagacctgagagcgctgggcccacagcctcccctcagagccctgctgcctcctccggtcagccctggacatcccaggtttccccaggcctggcggtaggtttagaatgaggtctgtgtcactgtggtattacgatattttgactggttattataaccacagtgtcacagagtccatcaaaaacccatgcctggaagcttcccgccacagccctccccatggggccctgctgcctcctcaggtcagccccggacatcccgggtttccccaggctgggcggtaggtttggggtgaggtctgtgtcactgtggtattactatggttcggggagttgtacggccctgatacttagcttccattgtatacatttttaaagtgatataaaacaaatctggttgtgattcctatcaacataggcatgagccactgcgctcagccctctttgtttttgtgctggttcctttgtgaaagttctgctgttga

SEQ ID NO: 54 - DNA, 20 bp, Artificial Sequence
  synthetic PCR/sequencing primer MYC
ggtcggacattcctgcttta

SEQ ID NO: 55 - DNA, 19 bp, Artificial Sequence
  synthetic PCR/sequencing primer IgH
cccagaaggagcaagatgg

SEQ ID NO: 56 - DNA, 545 bp, Artificial Sequence
  synthetic OCI-Ly8 Sgamma2-MYC fusion
gggtttggggggctgggggttgctttgcggtgggcagaaagccccttgcatcctgagctccttggagtagggaccgcatatcgcctgtgtgagccagattgctccgcagccgctgacttgtccccgtctccgggagggcatttaaatttcggctcaccgcatttctgacagccggagacggacactgcggcgcgtcccgcccgcctgtccccgcggcaaggtccctgttgggctcaacccaggccccccagcatatgtaggagccttgtatggccctccccaccctgcgtggtgccaggacccccaggccacagggaggccccatttctctctgccgctggcccagtggccctggagtcccactccacgtggggtgtgcccctgacttctgaggaacctaagtgccctgccctcagccaggccatcccctctgctcagagggccccgctcaccaccccttcccctcacctgcagcacagactctggctgattctgcccaggccctgaatgggcccctctggcagccgtctgttgctacactgccc

SEQ ID NO: 57 - DNA, 30 bp, Artificial Sequence
  synthetic PCR/sequencing primer IgH
cctgcctcccagtgtcctgcattacttctg

SEQ ID NO: 58 - DNA, 20 bp, Artificial Sequence
  synthetic PCR/sequencing primer BCL6
gcagtggtaaagtccgaagc

SEQ ID NO: 59 - DNA, 721 bp, Artificial Sequence
  synthetic breakpoint sequence for OCI-Ly8 Sgamma2-BCL6 (SgammaF) fusion
actggggttcttaaagtggtgatgcaagaagtttctaggaaaggccggacaccaggtgattattgctgttgctgccgccgctgctgctgctactgccgccgccgccgctgttgccgctggtgccgctgccgccgccgctgctcatgatcattattttaccttttaattctttttttttccgctcttgccgagtgctttggctccaagttttctatgtgtatctattgatataaatgtatatatttatttattctaggtggagctcaragagccggggaaratcatcggtaggtgagcaggggctggtggaaagcaggaggagcaaggggcagctcatggagctcagaggaccagggaagagcagccacaggtgaacaggggcaggtgggcggcagaatgagcaggggcaactcctggagctcaggggacaagggcagagcagccataggcaaacaggagcagggtcaggggacaggaggagcagggggcagttcttggagtttaggggaccagggcagagccgctgcaggtgagcaggggcaggtggggggcaggaggagcagggggcatctcctggagctcagagcaccagggcagagcagccacaggtgagcagggacggtgggaggcagcacgcagctcctagactttggcaggagctgggtagttgccggcaccagacagctgagggctggtgaaagtgcagtgcagcctcctggtg

SEQ ID NO: 60 - DNA, 20 bp, Artificial Sequence
  synthetic PCR/sequencing primer
tcagacaccagctccctagc

SEQ ID NO: 61 - DNA, 20 bp, Artificial Sequence
  synthetic PCR/sequencing primer
gaagcacagatggttgatgg

SEQ ID NO: 62 - DNA, 255 bp, Artificial Sequence
  synthetic breakpoint sequence for OCI-Ly8 intronic BCL2 deletion
aggaagtcttgctcttcagcaaaaactgcagcaggaatccctaaatgcctaaaatgacttttagattaataatgcacatttgaaatgataggcaaatcattcatcattctcaaacactaccacttttattccaaagtgtcctatgagtgcatttcctggtatatatatatattttttctctaaattcctttgttttcctatatagtcaccatctatgcattgttctgaaagctattacatattttaccatcaacc

SEQ ID NO: 63 - DNA, 22 bp, Artificial Sequence
  synthetic PCR/sequencing primer
ctcatgccccatattcattcaa

SEQ ID NO: 64 - DNA, 22 bp, Artificial Sequence
  synthetic PCR/sequencing primer
agggcatgttactgcaagttca

SEQ ID NO: 65 - DNA, 412 bp, Artificial Sequence
  synthetic breakpoint sequence for RL7 intronic BCL2 deletion
atttgtttactgtaaagagcctactctgggccaatcattgttctaggggctggagctgtaacagaaaacatgacaggttaagaactctcccatctcttgtgaatcccacattgtgaggaagtcagaaaagtaaacaacttaaaaattaacaatataacataagggccacaaagtaatgaaaacagggcaatgcagtgcaagggtgctgatgggacaatattaacttcttccttggtctttgagatttttcaagcagtactacaagtttacacagaggagatttaatgggtttttcttcattaatagttgaaaactatttataagacaaataataatttgtcttagggtctgtttctaagggagtctaacctatggccatgggatttactgttgaatgaatatggggcatgag